(19) United States
(12) Patent Application Publication
(10) Pub. No.: US 2026/0306491 A1
(43) Pub. Date: Oct. 8, 2026

(54) EGFR INHIBITOR FOR TREATING CANCERS COMPRISING ATYPICAL EGFR MUTATIONS

(71) Applicant: ORIC Pharmaceuticals, Inc., South San Francisco, CA (US)

(72) Inventors: Melissa R. JUNTTILA, San Mateo, CA (US); Anneleen DAEMEN, Belmont, CA (US); Pratik Sharad MULTANI, San Diego, CA (US); Edna Chow MANEVAL, Del Mar, CA (US); Rupal PATEL, San Diego, CA (US)

(21) Appl. No.: 19/481,494

(22) PCT Filed: May 3, 2024
(86) PCT No.: PCT/US2024/027642

§ 371 Date: Nov. 4, 2025

Related U.S. Application Data
(60) Provisional application No. 63/464,424, filed on May 5, 2023.
(60) Provisional application No. 63/591,656, filed on Oct. 19, 2023.

(51) Int. Cl. A61K 31/506; A61K 45/06; A61P 35/00; A61P 35/04

(52) U.S. Cl. CPC A61K 31/506; A61K 45/06; A61P 35/00; A61P 35/04

(57) ABSTRACT

The present application provides methods of treating cancer in an individual in need thereof, wherein in the cancer in the individual has been determined to comprise one or more atypical epidermal growth factor receptor (EGFR) mutations, comprising administering to the individual (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

### CROSS-REFERENCE TO RELATED APPLICATIONS

This application claims the benefit of U.S. Provisional Application No. 63/464,424 filed May 5, 2023 and U.S. Provisional Application No. 63/591,656 filed Oct. 19, 2023, the entire contents of each of which are hereby incorporated by reference.

### FIELD

Provided herein are methods of treating cancer in individuals, wherein the cancer has been determined to comprise one or more atypical epidermal growth factor receptor (EGFR) mutations, comprising administering to the individual (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide (hereinafter referred to as “Compound I”), or a pharmaceutically acceptable salt thereof. Also provided herein are such methods that utilize salt and crystalline forms of Compound I, and compositions comprising such salt and crystalline forms of Compound I.

### BACKGROUND

L858R mutations and exon 19 deletions represent the most common, classical mutations in the epidermal growth factor receptor (EGFR) protein in individuals having cancer, including non-small-cell lung cancer (NSCLC), treated with EGFR-tyrosine kinase inhibitors (TKIs) in first line therapy. Treatment of such individuals with some TKIs may result in the development of further mutations in the EGFR protein, which are known to those of ordinary skill in the art as atypical EGFR mutations. It is estimated that atypical EGFR mutations may comprise 10%-30% of EGFR-mutated NSCLC, either alone or in combination with other EGFR mutations. There are no clear established guidelines for EGFR TKI treatment for patients with atypical EGFR mutations in the absence of approved TKIs, often resulting in individuals having such mutations receiving chemotherapy. Therefore, there is an unmet need to develop treatments for individuals having atypical EGFR mutations.

Compound I is an inhibitor of epidermal growth factor receptor (EGFR) and has use in the treatment of various cancers in individuals, including humans, in need thereof. It has recently been discovered that Compound I can inhibit the activity of the EGFR protein comprising one or more atypical EGFR mutations. The present disclosure relates to methods of treating cancer in individuals, wherein the cancer has been determined to comprise one or more atypical epidermal growth factor receptor (EGFR) mutations, comprising administering to the individual Compound I, or a pharmaceutically acceptable salt thereof. Also provided herein are such methods that utilize salt and crystalline forms of Compound I, and/or compositions comprising such salt and crystalline forms of Compound I.

### SUMMARY

Provided herein are methods of treating cancer in an individual in need thereof, wherein in the cancer in the individual has been determined to comprise one or more atypical epidermal growth factor receptor (EGFR) mutations, comprising administering to the individual (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide (“Compound I”), or a pharmaceutically acceptable salt thereof. In one embodiment is provided such methods, wherein the one or more atypical epidermal growth factor receptor (EGFR) mutations in the cancer of the individual comprises one or more (a) P-loop and alpha C-helix compressing (PACC) EGFR mutations, (b) Exon18 mutations, (c) Exon19 mutations, (d) Exon20 point mutations, (e) Exon21 mutations, (f) mutations in the extracellular domain of EGFR, (g) mutations in the transmembrane domain of EGFR, and/or (h) Exon20 insertion mutations, provided that the Exon20 insertion mutations do not include EGFR_Exon20insNPH, EGFR_Exon20insSVD, EGFR_Exon20insFQEA, EGFR_Exon20insH, and EGFR_Exon20insASV, EGFR_Exon20insYVMA. In one embodiment is provided such methods, wherein the one or more atypical epidermal growth factor receptor (EGFR) mutations in the cancer of the individual is selected from the group consisting of EGFR_A702T, EGFR_E709A, EGFR_E709A G719A, EGFR_E709A G719S, EGFR_E709K, EGFR_E709K G719S, EGFR_E709_T710delinsD, EGFR_E709_T710del insD S22R, EGFR_L718Q, EGFR L718Q L858R, EGFR_L718V, EGFR_L718V L858R, EGFR_G719A, EGFR_G719A D761Y, EGFR_G719A L861Q, EGFR_G719A R776C, EGFR G719A S768L, EGFR G719C, EGFR G719C S768L, EGFR G719S, EGFR G719S L861Q, EGFR_T725M, EGFR_G719S S768L, EGFR_S720P, EGFR_E736K, EGFR_G724S, EGFR_G724S Exl9del, EGFR_G724S L858R, EGFR_I740dupIPVAK, EGFR_E746_A750del A647T, EGFR_E746_A750del G724S, EGFR_E746_A750del S768L, EGFR E746_A750del R675W, EGFR E746 T751del insV, EGFR E746 T751del insV S768C, EGFR_T751_I759 delinsN, EGFR_L747P, EGFR_L747S, EGFR_L747S V774M, EGFR_L747S L858R, EGFR L747 S752del A755D, EGFR_A750_I759del insPN, EGFR_S752_I759del V769M, EGFR_K757M L858R, EGFR_K757R, EGFR_D761N, EGFR_A767dupASV, EGFR_S768C, EGFR_S768I, EGFR_S768I V769L, EGFR_S768I V774M, EGFR_S768I L858R, EGFR_S768I L861Q, EGFR_S768dupSVD, EGFR_V769L, EGFR_V769M, EGFR_N771G, EGFR_H773dup, EGFR_V774M, EGFR_R776C, EGFR_R776H, EGFR_G779F, EGFR_L792H, EGFR_Exl9del L792H, EGFR_G796S, EGFR_V774M, EGFR_S784F, EGFR_Exl9del G796S, EGFR_L833V, EGFR_V834L, EGFR_T854I, EGFR_Exl9del T854L, EGFR_L858R L792H, EGFR_L858R C797S, EGFR_L858R T854S, EGFR_L861Q, EGFR_L861R, EGFR_S811F, EGFR_A763insFQEA, EGFR_A763insLQEA, EGFR_E746_A750delL41W, EGFR_E746_A750delR451H, EGFR_K754E, EGFR_L747_E749del, EGFR_A750P, EGFR_L747_T751del, and EGFR_L833F.

In another embodiment is provided such methods, wherein the cancer in the individual has been determined to further comprise one or more classical EGFR mutations. In another embodiment is provided such methods, wherein the cancer in the individual has been determined to further comprise one or more mutations in EGFR selected from the group consisting of G309A, G309E, S310F, R678Q, R678Q and L755W, L755S, L755W, I767M, D769H, D769Y, V777L, Y835F, V842I, R896C, G1201V, del.755-759, EGFR Del19/T790M, EGFR L858R/T790M, EGFR L858R, EGFR del 19, EGFR_del746-750, EGFR L858R/C797S, EGFR del19/C797S, EGFR Exon20 ins NPH, EGFR Exon20 ins SVD, EGFR Exon20 ins FQEA, EGFR Exon20 ins H, and EGFR Exon20 ins ASV.

In another embodiment is provided such methods, wherein the cancer has been determined by use of an FDA-approved test to comprise one or more atypical epidermal growth factor receptor (EGFR) mutations.

In another embodiment is provided such methods, wherein Compound I administered to the individual in the form of a salt. In another embodiment is provided such methods, wherein Compound I is administered to the individual in the form of a fumarate, hemi-fumarate, glycolate or malonate salt.

### DESCRIPTION OF THE DRAWINGS

FIG. 1A shows an experimental X-ray powder diffraction (XRPD) pattern of a malonate salt of Compound I as prepared according to Example 1.

FIG. 1B shows an experimental X-ray powder diffraction (XRPD) pattern of a malonate salt of Compound I as prepared according to Example 2.

FIG. 1C shows an experimental X-ray powder diffraction (XRPD) pattern of a malonate salt of Compound I as prepared according to Example 3.

FIG. 1D shows differential scanning calorimetry (DSC) and thermogravimetric analysis (TGA) graphs of a malonate salt of Compound I as prepared according to Example 1.

FIG. 1E shows differential scanning calorimetry (DSC) and thermogravimetric analysis (TGA) graphs of a malonate salt of Compound I as prepared according to Example 2.

FIG. 1F shows differential scanning calorimetry (DSC) and thermogravimetric analysis (TGA) graphs of a malonate salt of Compound I as prepared according to Example 3.

FIG. 1G shows a proton nuclear magnetic resonance (¹H NMR) spectrum of a malonate salt of Compound I as prepared according to Example 1.

FIG. 1H shows a proton nuclear magnetic resonance (¹H NMR) spectrum of a malonate salt of Compound I as prepared according to Example 2.

FIG. 1I shows a proton nuclear magnetic resonance (¹H NMR) spectrum a malonate salt of Compound I as prepared according to Example 3.

FIG. 1J shows a dynamic vapor sorption (DVS) isotherm plot of a malonate salt of Compound I as prepared according to Example 1.

FIG. 2A shows an experimental X-ray powder diffraction (XRPD) pattern of a glycolate salt of Compound I.

FIG. 2B shows differential scanning calorimetry (DSC) and thermogravimetric analysis (TGA) graphs of a glycolate salt of Compound I.

FIG. 2C shows a proton nuclear magnetic resonance (¹H NMR) spectrum of a glycolate salt of Compound I.

FIG. 2D shows a dynamic vapor sorption (DVS) isotherm plot of a glycolate salt of Compound I.

FIG. 3 provides the bioavailability and concentrations of unbound tucatinib and Compound I in the brain or plasma at 1 hour, 4 hours, and 8 hours following oral administration of a single dose of either tucatinib (50 mg/kg) or Compound I (3 mg/kg) into mice.

FIG. 4 shows the mean unbound plasma concentration of Compound I at steady state from dosing cycle 2, day 1 in human subjects administered Compound I as described in Example 10.

### DETAILED DESCRIPTION

### Definitions

Unless specifically indicated otherwise, all technical and scientific terms used herein have the same meaning as commonly understood by those of ordinary skill in the art to which this application belongs. In addition, any method or material similar or equivalent to a method or material described herein can be used in the practice of the present application. For purposes of the present application, the following terms are defined.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

The term “about X-Y” used herein has the same meaning as “about X to about Y.” The expression “about X, Y and/or Z” used herein has the same meaning as “about X, about Y, and/or about Z.”

As used herein, reference to “not” a value or parameter generally means and describes “other than” a value or parameter. For example, the method is not used to treat cancer of type X means the method is used to treat cancer of types other than X.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

As used herein, the term “atypical epidermal growth factor receptor (EGFR) mutation” means a mutation in the amino acid sequence of the EGFR protein, provided that atypical EGFR mutations do not include any of the following mutations alone: EGFR_T790M, EGFR_L858R, exon19 deletions, EGFR_Del19/T790M, EGFR L858R/T790M, EGFR Exon20insNPH, EGFR Exon20insSVD, EGFR_Exon20insFQEA, EGFR_Exon20insH, and EGFR_Exon20insASV, EGFR_Exon20insYVMA, EGFR_(E746-A750del), EGFR_G719C, EGFR_G719S, EGFR_(L747-E749del, A750P), EGFR_(L747-T751del, Sins), EGFR_(L861Q), and EGFR_(S752-1759del). Atypical epidermal growth factor receptor (EGFR) mutations include one or more (a) one or more P-loop and alpha C-helix compressing (PACC) EGFR mutations, (b) Exon18 mutations, (c) Exon19 mutations, (d) Exon20 point mutations, (e) Exon21 mutations, (f) mutations in the extracellular domain of EGFR, (g) mutations in the transmembrane domain of EGFR, and/or (h) Exon20 insertion mutations, provided that the Exon20 insertion mutations do not include EGFR_Exon20insNPH, EGFR_Exon20insSVD, EGFR_Exon20insFQEA, EGFR_Exon20insH, and EGFR_Exon20insASV, EGFR_Exon20insYVMA. Exemplary atypical EGFR mutations include, but are not limited to, EGFR_A702T, EGFR E709A, EGFR E709A G719A, EGFR E709A G719S, EGFR_E709K, EGFR_E709K G719S, EGFR_E709_T710delinsD, EGFR_E709_T710del insD S22R, EGFR_L718Q, EGFR L718Q L858R, EGFR_L718V, EGFR_L718V L858R, EGFR_G719A, EGFR_G719A D761Y, EGFR_G719A L861Q, EGFR_G719A R776C, EGFR G719A S768L, EGFR G719C, EGFR G719C S768L, EGFR G719S, EGFR G719S L861Q, EGFR_T725M, EGFR_G719S S768L, EGFR_S720P, EGFR_E736K, EGFR_G724S, EGFR_G724S Exl9del, EGFR_G724S L858R, EGFR_I740dupIPVAK, EGFR_E746_A750del A647T, EGFR_E746_A750del G724S, EGFR_E746_A750del S768L, EGFR E746_A750del R675W, EGFR E746 T751del insV, EGFR E746 T751del insV S768C, EGFR_T751_I759 delinsN, EGFR_L747P, EGFR_L747S, EGFR_L747S V774M, EGFR_L747S L858R, EGFR_L747_S752del A755D, EGFR_A750_I759del insPN, EGFR_S752_I759del V769M, EGFR_K757M L858R, EGFR_K757R, EGFR_D761N, EGFR_A767dupASV, EGFR_S768C, EGFR_S768I, EGFR_S768I V769L, EGFR_S768I V774M, EGFR_S768I L858R, EGFR_S768I L861Q, EGFR_S768dupSVD, EGFR_V769L, EGFR_V769M, EGFR_N771G, EGFR_H773dup, EGFR_V774M, EGFR_R776C, EGFR_R776H, EGFR_G779F, EGFR_L792H, EGFR_Exl9del L792H, EGFR_G796S, EGFR V774M, EGFR S784F, EGFR Exl9del G796S, EGFR L833V, EGFR V834L, EGFR_T854I, EGFR_Exl9del T854L, EGFR_L858R L792H, EGFR_L858R C797S, EGFR_L858R T854S, EGFR_L861Q, EGFR_L861R, EGFR_S811F, EGFR_A763insFQEA, EGFR_A763insLQEA, EGFR_E746_A750delL41W, EGFR_E746_A750delR451H, EGFR_K754E, EGFR_L747_E749del, EGFR_A750P, EGFR_L747_T751del, and EGFR_L833F. Further exemplary atypical EGFR mutations are disclosed in Table 13.

TABLE 13

| Atypical EGFR | Atypical EGFR | Atypical EGFR | Atypical EGFR |
|---|---|---|---|
| Mutation | Mutation | Mutation | Mutation |
| 709E > A, 719G > A | 773Hdup | 742V > F | 874G > A |
| 709E > A, 719G > C | 773_774HVinsH | 742V > I | 689V > M, |
| | | | 747_751LTdel |
| 709E > A, 719G > S | 773Hdup3 | 743A > S | 713K > T, 858L > R |
| 709E > K, 719G > A | 774VdelinsGL | 744I > M | 718L > Q, 858L > R |
| 709E > A, 858L > R | 774VdelinsPHPHV | 744I > V | 718L > V, 858L > R |
| 709E > G, 858L > R | 787_790QTdup | 747L > P | 724G > S, |
| | | | 746_751ETdelinsIP |
| 709E > K, 858L > R | 770_771DNinsSVD | 748R > T | 724G > S, |
| | | | 746_752ESdelinsV |
| 709E > V, 858L > R | 770_771DNinsY | 749E > G | 724G > S, |
| | | | 752_759SIdel |
| 719G > A | 770_772DPdup | 751T > A | 725T > M, 858L > R |
| 719G > C | 770_773DHdup | 751T > I | 738V > F, 858L > R |
| 719G > S | 770_774DVdup | 753P > L | 746_750EAdel, |
| | | | 769V > M |
| 719G > X | 770DdelinsGY | 755A > V | 746_750EAdel, |
| | | | 848P > L |
| 719G > A, 768S > I | 770DdelinsNNN | 758E > G | 757K > M, 858L > R |
| 719G > C, 768S > I | 771_772NPinsH | 761D > Y | 759I > M, 858L > R |
| 719G > D, 768S > I | 771_772NPinsPAN | 765V > M | 769V > M, 858L > R |
| 719G > R, 768S > I | 771_773NHdup | 768S > N | 776R > G, 858L > R |
| 719G > S, 768S > I | 771NdelinsGF | 769V > A | 776R > H, 858L > R |
| 719G > A, 861L > Q | 771NdelinsGY | 769V > M | 791Q > L, 858L > R |
| 719G > D, 861L > Q | 771NdelinsH | 770D > G | 817W > F, 858L > R |
| 719G > S, 861L > Q | 771NdelinsKG | 772P > S | 834V > L, 858L > R |
| 768S > I | 771NdelinsKPHPH | 773H > Y | 843V > I, 858L > R |
| 768S > N | 771NdelinsTH | 774V > L | 854T > A, 858L > R |
| 747_751LTdel, | 771Ndup | 776R > C | 858L > R, 859A > S |
| 768S > I | | | |
| 768S > I, 858L > R | 772_773PHdup | 776R > H | 858L > R, 860K > I |
| 861L > Q | 772_773PHinsGNP | 776R > L | 858L > R, 871A > G |
| 861L > R | 772_773PHinsHV | 779G > F | 706I > T, 719G > S |
| 762_765EVdel | 772_773PHinsPHP | 779G > V | 707L > W, 719G > A |
| 763_764AYinsFQEV | 772_773PHinsPR | 768V > A | 709E > K, 719G > S, |
| | | | 757K > N |
| 767_768ASinsAWT | 772_773PHinsQ | 788L > F | 714K > N, 719G > R |
| 767_768ASinsTLA | 772_773PHinsRNP | 789I > V | 719G > A, 744I > M |
| 767_769AVdup | 772_773PHinsTGNP | 791Q > H | 719G > A, 753P > Q |
| 767_769AVdup3 | 772PdelinsTP | 791Q > R | 719G > A, 756N > I, |
| | | | 861L > Q |
| 768_769SVdelinsIL | 773_774HVdelinsLM | 794P > S | 719G > A, 761D > Y |
| 768_769SVdelinsPL | 773_774HVdup | 796G > S | 719G > A, 776R > H |
| 768_770SDdup | 773_774HVinsAH | 798L > F | 719G > A, |
| | | | 833_834LVdelinsFL |
| 767_768ASinsSVD | 773_774HVinsGHPH | 801Y > C | 719G > A, 834V > L |
| 769_770VDinsCV | 773_774HVinsGNPH | 803R > L | 719G > C, 776R > H |
| 769_770VDinsSSV | 773_774HVinsHNPH | 806K > R | 719G > C, 864A > P |
| 769_771VNdup | 773_774HVinsT | 808N > S | 719G > S, 776R > H |
| 770_771DNdelinsP | 773HdelinsNPNPY | 810G > F | 719G > S, 779G > C |
| 770_771DNinsG | 773Hdup | 812Q > L | 719G > V, 747L > P |
| 770_771DNinsGF | 773_774HVinsH | 816N > I | 779G > C, 861L > Q |
| 770_771DNinsGMD | 773Hdup3 | 816N > S | 833L > F, 861L > Q |
| 770_771DNinsNPG | 774VdelinsGL | 816N > Y | 724G > S, 768S > I |
| 770_771DNinsNPH | 774VdelinsPHPHV | 821I > V | 768S > I, 769V > L |
| 770_771DNinsSVD | 787_790QTdup | 822A > P | 768S > I, 774V > M |
| 770_771DNinsY | 690E > K | 823K > M | 713K > T, |
| | | | 771_773NHdup |
| 770_772DPdup | 691G > S | 823K > N | 719G > A, |
| | | | 768_770SDdup, |
| 770_773DHdup | 693T > I | 824G > C | 735G > S, |
| | | | 767_769AVdup |
| 770_774DVdup | 694P > S | 824G > R | 767_769AVdup, |
| | | | 770D > G |
| 770DdelinsGY | 699P > S | 830D > Y | 699P > L, 854T > I |
| 770DdelinsNNN | 701Q > K | 831R > H | 735G > S, 741P > T |
| 771_772NPinsH | 702A > T | 832R > H | 735G > S, 751T > M |
| 771_772NPinsPAN | 704L > F | 833L > F | 741P > S, 848P > L |
| 771_773NHdup | 704L > W | 834V > M | 746E > K, 864A > V |
| 771NdelinsGF | 708K > N | 836R > H | 773H > L, 774V > M |
| 771NdelinsGY | 710T > I | 837D > N | 796G > S, 804E > S |
| 771NdelinsH | 711E > K | 837D > Y | 833L > V, 835H > L |
| 771NdelinsKG | 711E > Q | 838L > R | 709_710ETdelinsA |
| 771NdelinsKPHPH | 713K > T | 840A > S | 709_710ETdelinsD |
| 771NdelinsTH | 713K > Y | 840A > T | 709_710ETdelinsN |
| 771Ndup | 720S > A | 843V > L | 738_739VKinsKIPVAI |
| 772_773PHdup | 720S > C | 844L > V | 739_744KIdup |
| 772_773PHinsGNP | 720S > F | 848P > A | same as |
| | | | 738_739VKinsKIPVAI, |
| | | | 740_745IKdup |
| 772_773PHinsHV | 721G > C | 848P > L | 740_745IKdup |
| 772_773PHinsPHP | 722A > S | 855D > N | 741_745PKdupinsV |
| 772_773PHinsPR | 722A > V | 857G > E | 744_749IEdelinsMKK |
| 772_773PHinsQ | 725T > A | 858L > A | 745_746KEinsIPVAIK |
| 772_773PHinsRNP | 725T > M | 859A > P | 745_746KEinsVPVAIK |
| 772_773PHinsTGNP | 726V > M | 859A > S | 746_747ELdelinsIP, |
| | | | 747_750LAdelinsS |
| 772PdelinsTP | 727Y > C | 859A > T | 746_747ELdelinsIP, |
| | | | 747_751LTdelinsS |
| 773_774HVdelinsLM | 728K > N | 860_861KLdelinsVW | 746_748ERdel, |
| | | | 754K > E |
| 773_774HVdup | 730L > R | 863G > V | 746_749EEdel, |
| | | | 751T > A |
| 773_774HVinsAH | 731W > S | 864A > T | 746E > V, |
| | | | 746_752ESdelinsD |
| 773_774HVinsGHPH | 733P > Q | 865E > K | 747_749LEdel, |
| | | | 754K > E, |
| 773_774HVinsGNPH | 734E > K | 866E > D | 747_753LPdelinsS, |
| | | | 755A > G, |
| 773_774HVinsHNPH | 734E > V | 867K > T | 751T > K, |
| | | | 751_759TIdelinsT |
| 773_774HVinsT | 735G > S | 872E > D | 751T > N, |
| | | | 751_759TIdelinsT |
| 773HdelinsNPNPY | 741P > L | 873G > E | 754K > E, |
| | | | 747_749LEdel |

It is specifically contemplated that in certain embodiments of the methods disclosed herein, the cancer in the individual in need thereof has been determined to comprise one or more (i) atypical EGFR mutations, and (ii) one or more classical EGFR mutations. It is further contemplated that in certain embodiments of the methods disclosed herein, the cancer in the individual in need thereof has been determined to comprise one or more (i) atypical EGFR mutations, and (ii) one or more EGFR mutations selected from EGFR_T790M, EGFR_L858R, exon19 deletions, EGFR_Del19/T790M, EGFR_L858R/T790M, EGFR_Exon20insNPK. EGFR_Exon20insSVD, EGFR_Exon20insFQEA. EGFR_Exon20insH, and EGFR_Exon20insASV, EGFR_Exon20insYVMA, EGFR_(E746-A750del), EGFR_G719C, EGFR_G719S, EGFR_(L747-E749del, A750P), EGFR_(L747-T751del, Sins), EGFR_(L861Q), and EGFR_(S752-1759del). For example, the cancer in the individual in need thereof may comprise (i) one or more atypical EGFR mutations, and (ii) one or more mutations selected from (a) EGFR_T790M, (b) EGFR_T790M, (c) EGFR_L858R, (d) exon19 deletion, (e) EGFR_Del19/T790M, (f) EGFR_L858R/T790M, (g) EGFR_Exon20insNPH, (h) EGFR_Exon20insSVD, (i) EGFR_Exon20insFQEA, (j) EGFR_Exon20insH, (k) EGFR_Exon20insASV, (l) EGFR_Exon20insYVMA, (m) EGFR_(E746-A750del), (n) EGFR_G719C, (o) EGFR_G719S, (p) EGFR_(L747-E749del, A750P), (q) EGFR_(L747-T751del, Sins), (r) EGFR_(L861Q), and (s) EGFR_(S752-1759del).

As used herein, the term “the cancer in the individual has been determined to comprise one or more atypical epidermal growth factor receptor (EGFR) mutations” means that a biological sample (as described further herein) from the individual having cancer has been tested and has established the presence of one or more atypical mutations in the EGFR protein in the biological sample prior to administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof. The types of tests that may be used to determine the presence (or absence) of one or more atypical mutations in the EGFR protein in a biological sample from an individual having cancer are known to those having ordinary skill in the art, and are also described further herein. In some embodiments, the test used to determine the presence (or absence) of one or more atypical mutations in the EGFR protein in a biological sample from an individual having cancer is an FDA-approved test. In some embodiments, if a test of a biological sample from the individual having cancer is conducted and such test does not detect the presence of one or more atypical mutations in the EGFR protein in the biological sample, then (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, would not be administered to the individual.

As used herein, the term “polymorph” or “polymorphic form” refers to a crystalline form of a compound. Different polymorphs may have different physical properties such as, for example, melting temperatures, heats of fusion, solubilities, dissolution rates, and/or vibrational spectra as a result of the arrangement or conformation of the molecules or ions in the crystal lattice. The differences in physical properties exhibited by polymorphs may affect pharmaceutical parameters, such as storage stability, compressibility, density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph), mechanical changes (e.g., tablets crumble on storage as a kinetically favored polymorph converts to thermodynamically more stable polymorph), or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of a crystalline form may be important in processing; for example, one polymorph might be more likely to form solvates or might be difficult to filter and wash free of impurities (e.g., particle shape and size distribution might be different between polymorphs).

As used herein, the term “substantially as shown in” when referring, for example, to an XRPD pattern, a DSC graph, a TGA graph, or a GVS graph, includes a pattern or graph that is not necessarily identical to those depicted herein, but that falls within the limits of experimental error or deviations when considered by one of ordinary skill in the art.

In some embodiments, the term “substantially pure” means that the salt form or polymorphic form contains about less than 30%, about less than 20%, about less than 15%, about less than 10%, about less than 5%, or about less than 1% by weight of impurities. In other embodiments, “substantially pure” refers to a substance free of impurities. Impurities may, for example, include by-products or left over reagents from chemical reactions, contaminants, degradation products, other salt forms, other polymorphic forms, water, and solvents.

As used herein, the term “substantially free of” means that the composition comprising the salt form or polymorphic form contains less than 50%, less than 40%, less than 30%, less than 20%, less than 15%, less than 10%, less than 5%, less than 4%, less than 3%, less than 2%, or less than 1% by weight of the indicated substance or substances.

As used herein, “treatment” or “treating” is an approach for obtaining beneficial or desired results including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In some embodiments, the treatment reduces the severity of one or more symptoms associated with cancer by at least about any of 10%, 20%, 30%, 40%, 50%, 60%, 70%, 80%, 90%, 95% or 100% compared to the corresponding symptom in the same subject prior to treatment or compared to the corresponding symptom in other subjects not receiving the treatment. Also encompassed by “treatment” is a reduction of pathological consequence of cancer. The methods of the application contemplate any one or more of these aspects of treatment.

The term “effective amount” used herein refers to an amount of a compound or composition sufficient to treat a specified disorder, condition, or disease, such as to ameliorate, palliate, lessen, and/or delay one or more of its symptoms in an individual. In reference to cancer, an effective amount comprises an amount sufficient to cause a tumor to shrink and/or to decrease the growth rate of the tumor (such as to suppress tumor growth) or to prevent or delay other unwanted cell proliferation in cancer in an individual. In some embodiments, an effective amount is an amount sufficient to delay development of cancer in an individual. In some embodiments, an effective amount is an amount sufficient to prevent or delay recurrence of cancer in an individual. In some embodiments, an effective amount is an amount sufficient to reduce recurrence rate of cancer in an individual. An effective amount can be administered to an individual in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer in an individual.

As is understood in the art, an “effective amount” may be in one or more doses, i.e., a single dose or multiple doses may be required to achieve the desired treatment endpoint. An effective amount may be considered in the context of administering one or more therapeutic agents, and a compound or composition described herein may be considered to be given in an effective amount if, optionally in conjunction with one or more other agents, a desirable or beneficial result may be or is achieved. The components (e.g., the first and second therapies) in a combination therapy of the application may be administered to an individual sequentially, simultaneously, or concurrently using the same or different routes of administration for each component. Thus, an effective amount of a combination therapy includes an amount of the first therapy and an amount of the second therapy that when administered sequentially, simultaneously, or concurrently to an individual produces a desired outcome in the individual.

As used herein, “therapeutically effective amount” indicates an amount administered to an individual that results in a desired pharmacological and/or physiological effect for the condition in the individual. The effect in the individual may be prophylactic in terms of completely or partially preventing a condition or symptom thereof and/or may be therapeutic in terms of a partial or complete cure for the condition and/or adverse effect attributable to the condition.

As used herein, “individual” means a mammal, including a human, dog, cat, or livestock. In one embodiment, individual means a human.

“In conjunction with” or “in combination with” refers to administration of one treatment modality in addition to another treatment modality, such as administration of a compound or composition described herein in addition to administration of the other agent to the same individual under the same treatment plan. As such, “in conjunction with” or “in combination with” refers to administration of one treatment modality before, during or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U. S. Food and Drug administration.

The disclosures of all publications, patents, patent applications and published patent applications referred to herein are hereby incorporated herein by reference in their entirety.

### Compound I

Compound I is a compound that has been demonstrated to have inhibitory activity against epidermal growth factor receptor (EGFR) variants. The chemical name of Compound I is N-[2-[4-(4-cyclopropyl-1-piperazinyl)-1-piperidinyl]-5-[[6-[(3R)-3-(3,5-difluorophenyl)-2-isoxazolidinyl]-4-pyrimidinyl]amino]-4-methoxyphenyl]-2-propenamide. Compound I has the following structure:

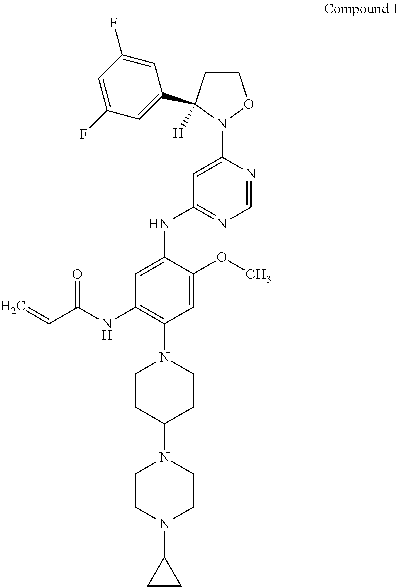 [SMILES: C=CC(=O)Nc1cc(Nc2cc(N3OCC[C@@H]3c3cc(F)cc(F)c3)ncn2)c(OC)cc1N1CCC(N2CCN(C3CC3)CC2)CC1]

Compound I can also be named as (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide. Compound I has been assigned CAS Registry No. 2489185-38-6. It is understood that descriptions herein to a salt or pharmaceutical composition provided herein refer to salts of Compound I, including pharmaceutically acceptable salts of Compound I, or pharmaceutical compositions comprising Compound I, or salts of Compound I, including pharmaceutically acceptable salts of Compound I.

Compound I has been described in WO2020/190119, and may be prepared by methods described therein. The contents of WO2020/190119 are hereby incorporated by reference herein for that purpose. Alternatively, Compound I may be prepared by methods known to those having ordinary skill in the art.

Provided herein are methods of treating cancer in an individual in need thereof, wherein in the cancer in the individual has been determined to comprise one or more atypical epidermal growth factor receptor (EGFR) mutations, comprising administering to the individual Compound I, or a pharmaceutically acceptable salt thereof. In another embodiment is provided such methods, wherein the individual is administered a therapeutically effective amount of Compound I, or a pharmaceutically acceptable salt thereof.

In another embodiment is provided such methods, wherein the cancer has been determined by use of an FDA-approved test to comprise one or more atypical epidermal growth factor receptor (EGFR) mutations.

In another embodiment is provides such methods, wherein the cancer is a metastatic solid tumor. In some variations, the cancer is cancer is a metastatic solid tumor and is unresectable. In some variations, the cancer is a metastatic solid tumor and comprises one or more central nervous system (CNS) metastases (e.g., brain metastases). In some variations, the cancer is a metastatic solid tumor and is selected from the group consisting of metastatic brain cancer, breast cancer, and non-small cell lung cancer. In some variations, the cancer is a metastatic solid tumor comprising brain cancer. In some variations, the cancer is a metastatic solid tumor comprising metastatic brain cancer. In some variations, the cancer is a metastatic solid tumor comprising breast cancer. In some variations, the cancer is a metastatic solid tumor comprising non-small cell lung cancer. In some variations, the cancer is metastatic non-small cell lung cancer. In some variations, the cancer is metastatic non-small cell lung cancer, wherein the cancer comprises central nervous system (CNS) metastases (e.g., brain metastases).

In another embodiment is provided such methods, wherein in the cancer in the individual has been determined by use of any nucleic acid-based diagnostic testing method to comprise one or more atypical epidermal growth factor receptor (EGFR) mutations. In another embodiment is provided such methods, wherein in the cancer in the individual is non-small cell lung cancer and the cancer has been determined by use of any nucleic acid-based diagnostic testing method to comprise one or more atypical epidermal growth factor receptor (EGFR) mutations.

In another embodiment is provided such methods, wherein in the cancer in the individual is locally advanced or metastatic non-small cell lung cancer with one or more atypical epidermal growth factor receptor (EGFR) mutations as detected by an FDA-approved test, and wherein the cancer has progressed on or after platinum-based chemotherapy. In another embodiment is provided such methods, wherein in the cancer in the individual is locally advanced or metastatic non-small cell lung cancer comprising one or more atypical epidermal growth factor receptor (EGFR) mutations as detected by an FDA-approved test, and wherein the cancer has progressed on or after platinum-based chemotherapy.

In another embodiment is provided such methods, wherein in the cancer in the individual is locally advanced or metastatic non-small cell lung cancer with one or more atypical epidermal growth factor receptor (EGFR) mutations, as detected by an FDA-approved test, and wherein the cancer has progressed following prior EGFR therapy.

In another embodiment is provided such methods, wherein the one or more atypical epidermal growth factor receptor (EGFR) mutations in the cancer of the individual comprises one or more (a) P-loop and alpha C-helix compressing (PACC) EGFR mutations, (b) Exon18 mutations, (c) Exon19 mutations, (d) Exon20 point mutations, (e) Exon21 mutations, (f) mutations in the extracellular domain of EGFR, (g) mutations in the transmembrane domain of EGFR, and (h) Exon20 insertion mutations, provided that the Exon20 insertion mutations do not include EGFR_Exon20insNPH, EGFR_Exon20insSVD, EGFR_Exon20insFQEA, EGFR_Exon20insH, and EGFR_Exon20insASV, EGFR_Exon20insYVMA.

In another embodiment is provided such methods, wherein the one or more atypical epidermal growth factor receptor (EGFR) mutations in the cancer of the individual is selected from EGFR_A702T, EGFR_E709A, EGFR_E709A G719A, EGFR_E709A G719S, EGFR E709K, EGFR E709K G719S, EGFR E709 T710delinsD, EGFR E709 T710del insD S22R, EGFR_L718Q, EGFR L718Q L858R, EGFR_L718V, EGFR_L718V L858R, EGFR_G719A, EGFR_G719A D761Y, EGFR_G719A L861Q, EGFR_G719A R776C, EGFR_G719A S768L, EGFR_G719C, EGFR_G719C S768L, EGFR_G719S, EGFR_G719S L861Q, EGFR_T725M, EGFR_G719S S768L, EGFR_S720P, EGFR_E736K, EGFR_G724S, EGFR_G724S Exl9del, EGFR_G724S L858R, EGFR_I740dupIPVAK, EGFR_E746_A750del A647T, EGFR_E746_A750del G724S, EGFR_E746_A750del S768L, EGFR_E746_A750del R675W, EGFR_E746_T751del insV, EGFR_E746_T751del insV S768C, EGFR T751_I759 delinsN, EGFR L747P, EGFR_L747S, EGFR_L747S V774M, EGFR_L747S L858R, EGFR_L747_S752del A755D, EGFR_A750_I759del insPN, EGFR_S752_I759del V769M, EGFR_K757M L858R, EGFR_K757R, EGFR_D761N, EGFR_A767dupASV, EGFR_S768C, EGFR_S768I, EGFR_S768I V769L, EGFR_S768I V774M, EGFR_S768I L858R, EGFR_S768I L861Q, EGFR_S768dupSVD, EGFR_V769L, EGFR_V769M, EGFR_N771G, EGFR_H773dup, EGFR_V774M, EGFR_R776C, EGFR_R776H, EGFR_G779F, EGFR_L792H, EGFR_Exl9del L792H, EGFR_G796S, EGFR_V774M, EGFR_S784F, EGFR_Exl9del G796S, EGFR_L833V, EGFR_V834L, EGFR T854I, EGFR Exl9del T854L, EGFR L858R L792H, EGFR L858R C797S, EGFR_L858R T854S, EGFR_L861Q, EGFR_L861R, EGFR_S811F, EGFR_A763insFQEA, EGFR_A763insLQEA, EGFR_E746_A750delL41W, EGFR_E746_A750delR451H, EGFR_K754E, EGFR_L747_E749del, EGFR_A750P, EGFR_L747_T751del, and EGFR_L833F. In one embodiment, the cancer in the individual has been determined to comprise an EGFR_A702T mutation. In another embodiment, the cancer in the individual has been determined to comprise an EGFR_E709A mutation. In another embodiment, the cancer in the individual has been determined to comprise an EGFR_E709A G719A mutation. In another embodiment, the cancer in the individual has been determined to comprise an EGFR_E709A G719S mutation. In another embodiment, the cancer in the individual has been determined to comprise an EGFR_E709K mutation. In another embodiment, the cancer in the individual has been determined to comprise an EGFR_E709K G719S mutation. In another embodiment, the cancer in the individual has been determined to comprise an EGFR_E709_T710delinsD mutation. In another embodiment, the cancer in the individual has been determined to comprise an EGFR_E709_T710del insD S22R mutation. In another embodiment, the cancer in the individual has been determined to comprise an EGFR_L718Q mutation. In another embodiment, the cancer in the individual has been determined to comprise an EGFR_L718Q L858R mutation. In another embodiment, the cancer in the individual has been determined to comprise an EGFR_L718V mutation. In another embodiment, the cancer in the individual has been determined to comprise an EGFR_L718V L858R mutation. In another embodiment, the cancer in the individual has been determined to comprise an EGFR_G719A mutation. In another embodiment, the cancer in the individual has been determined to comprise an EGFR_G719A D761Y mutation. In another embodiment, the cancer in the individual has been determined to comprise an EGFR_G719A L861Q mutation. In another embodiment, the cancer in the individual has been determined to comprise an EGFR_G719A R776C mutation. In another embodiment, the cancer in the individual has been determined to comprise an EGFR_G719A S768I mutation. In another embodiment, the cancer in the individual has been determined to comprise an EGFR_G719C mutation. In another embodiment, the cancer in the individual has been determined to comprise an EGFR_G719C S768I mutation. In another embodiment, the cancer in the individual has been determined to comprise an EGFR_G719S mutation. In another embodiment, the cancer in the individual has been determined to comprise an EGFR_G719S L861Q mutation. In another embodiment, the cancer in the individual has been determined to comprise an EGFR_T725M mutation. In another embodiment, the cancer in the individual has been determined to comprise an EGFR_G719S S768I mutation. In another embodiment, the cancer in the individual has been determined to comprise an EGFR_S720P mutation. In another embodiment, the cancer in the individual has been determined to comprise an EGFR_E736K mutation. In another embodiment, the cancer in the individual has been determined to comprise an EGFR_G724S mutation. In another embodiment, the cancer in the individual has been determined to comprise an EGFR_G724S Exl9del mutation. In another embodiment, the cancer in the individual has been determined to comprise an EGFR_G724S L858R mutation. In another embodiment, the cancer in the individual has been determined to comprise an EGFR_I740dupIPVAK mutation. In another embodiment, the cancer in the individual has been determined to comprise an EGFR_E746_A750del A647T mutation. In another embodiment, the cancer in the individual has been determined to comprise an EGFR_E746_A750del G724S mutation. In another embodiment, the cancer in the individual has been determined to comprise an EGFR_E746_A750del S768I mutation. In another embodiment, the cancer in the individual has been determined to comprise an EGFR_E746_A750del R675W mutation. In another embodiment, the cancer in the individual has been determined to comprise an EGFR_E746_T751del insV mutation. In another embodiment, the cancer in the individual has been determined to comprise an EGFR_E746_T751del insV S768C mutation. In another embodiment, the cancer in the individual has been determined to comprise an EGFR_T751_I759 delinsN mutation. In another embodiment, the cancer in the individual has been determined to comprise an EGFR_L747P mutation. In another embodiment, the cancer in the individual has been determined to comprise an EGFR_L747S mutation. In another embodiment, the cancer in the individual has been determined to comprise an EGFR_L747S V774M mutation. In another embodiment, the cancer in the individual has been determined to comprise an EGFR_L747S L858R mutation. In another embodiment, the cancer in the individual has been determined to comprise an EGFR_L747_S752del A755D mutation. In another embodiment, the cancer in the individual has been determined to comprise an EGFR_A750_I759del insPN mutation. In another embodiment, the cancer in the individual has been determined to comprise an EGFR_S752_I759del V769M mutation. In another embodiment, the cancer in the individual has been determined to comprise an EGFR_K757M L858R mutation. In another embodiment, the cancer in the individual has been determined to comprise an EGFR_K757R mutation. In another embodiment, the cancer in the individual has been determined to comprise an EGFR_D761N mutation. In another embodiment, the cancer in the individual has been determined to comprise an EGFR_A767dupASV mutation. In another embodiment, the cancer in the individual has been determined to comprise an EGFR_S768C mutation. In another embodiment, the cancer in the individual has been determined to comprise an EGFR_S768I mutation. In another embodiment, the cancer in the individual has been determined to comprise an EGFR_S768I V769L mutation. In another embodiment, the cancer in the individual has been determined to comprise an EGFR_S768I V774M mutation. In another embodiment, the cancer in the individual has been determined to comprise an EGFR_S768I L858R mutation. In another embodiment, the cancer in the individual has been determined to comprise an EGFR_S768I L861Q mutation. In another embodiment, the cancer in the individual has been determined to comprise an EGFR_S768dupSVD mutation. In another embodiment, the cancer in the individual has been determined to comprise an EGFR_V769L mutation. In another embodiment, the cancer in the individual has been determined to comprise an EGFR_V769M mutation. In another embodiment, the cancer in the individual has been determined to comprise an EGFR_N771G mutation. In another embodiment, the cancer in the individual has been determined to comprise an EGFR_H773dup mutation. In another embodiment, the cancer in the individual has been determined to comprise an EGFR_V774M mutation. In another embodiment, the cancer in the individual has been determined to comprise an EGFR_R776C mutation. In another embodiment, the cancer in the individual has been determined to comprise an EGFR_R776H mutation. In another embodiment, the cancer in the individual has been determined to comprise an EGFR_G779F mutation. In another embodiment, the cancer in the individual has been determined to comprise an EGFR_L792H mutation. In another embodiment, the cancer in the individual has been determined to comprise an EGFR_Exl9del L792H mutation. In another embodiment, the cancer in the individual has been determined to comprise an EGFR_G796S mutation. In another embodiment, the cancer in the individual has been determined to comprise an EGFR_V774M mutation. In another embodiment, the cancer in the individual has been determined to comprise an EGFR_S784F mutation. In another embodiment, the cancer in the individual has been determined to comprise an EGFR_Exl9del G796S mutation. In another embodiment, the cancer in the individual has been determined to comprise an EGFR_L833V mutation. In another embodiment, the cancer in the individual has been determined to comprise an EGFR_V834L mutation. In another embodiment, the cancer in the individual has been determined to comprise an EGFR_T854I mutation. In another embodiment, the cancer in the individual has been determined to comprise an EGFR_Exl9del T854I mutation. In another embodiment, the cancer in the individual has been determined to comprise an EGFR_L858R L792H mutation. In another embodiment, the cancer in the individual has been determined to comprise an EGFR_L858R C797S mutation. In another embodiment, the cancer in the individual has been determined to comprise an EGFR_L858R T854S mutation. In another embodiment, the cancer in the individual has been determined to comprise an EGFR_L861Q mutation. In another embodiment, the cancer in the individual has been determined to comprise an EGFR_L861R mutation. In another embodiment, the cancer in the individual has been determined to comprise an EGFR_S811F mutation. In another embodiment, the cancer in the individual has been determined to comprise an EGFR_A763insFQEA mutation. In another embodiment, the cancer in the individual has been determined to comprise an EGFR_A763insLQEA, mutation. In another embodiment, the cancer in the individual has been determined to comprise an EGFR_E746_A750delL41W mutation. In another embodiment, the cancer in the individual has been determined to comprise an EGFR_E746_A750delR451H mutation. In another embodiment, the cancer in the individual has been determined to comprise an EGFR_K754E mutation. In another embodiment, the cancer in the individual has been determined to comprise an EGFR_L747_E749del mutation. In another embodiment, the cancer in the individual has been determined to comprise an EGFR_A750P, mutation. In another embodiment, the cancer in the individual has been determined to comprise an EGFR_L747_T751del mutation. In another embodiment, the cancer in the individual has been determined to comprise an EGFR_L833F mutation.

In another embodiment, the cancer in the individual has been determined to comprise one or more EGFR mutations selected from those set forth in Table 13.

In another embodiment is provided such methods, wherein the cancer in the individual has been determined to further comprise an EGFR_C797S mutation.

In another embodiment is provided such methods, wherein the cancer in the individual has been determined not to comprise an EGFR_C797S mutation.

In another embodiment is provided such methods, wherein the cancer in the individual has been determined to further comprise one or more classical EGFR mutations.

In another embodiment is provided such methods, wherein the cancer in the individual has been determined to further comprise one or more EGFR mutation selected from T790M, L858R, one or more exon 19 deletions, and one or more exon 20 insertion mutations. In another embodiment is provided such methods, wherein the cancer in the individual has been determined to further comprise a T790M mutation. In another embodiment is provided such methods, wherein the cancer in the individual has been determined to further comprise an EGFR mutation L858R. In another embodiment is provided such methods, wherein the cancer in the individual has been determined to further comprise one or more EGFR mutation selected from one or more exon 19 deletions. In another embodiment is provided such methods, wherein the cancer in the individual has been determined to further comprise one or more EGFR mutation selected from one or more exon 20 insertion mutations.

In another embodiment is provided such methods, wherein the cancer in the individual has been determined to further comprise one or more mutations in EGFR selected from the group consisting of G309A, G309E, S310F, R678Q, R678Q and L755W, L755S, L755W, 1767M, D769H, D769Y, V777L, Y835F, V842I, R896C, G1201V, del.755-759, EGFR Del19/T790M, EGFR L858R/T790M, EGFR L858R, EGFR del 19, EGFR_de1746-750, EGFR L858R/C797S, EGFR del19/C797S, EGFR Exon20 ins NPH, EGFR Exon20 ins SVD, EGFR Exon20 ins FQEA, EGFR Exon20 ins H, and EGFR Exon20 ins ASV. In another embodiment is provided such methods, wherein the cancer in the individual has been determined to further comprise EGFR mutation G309A. In another embodiment is provided such methods, wherein the cancer in the individual has been determined to further comprise EGFR mutation G309E. In another embodiment is provided such methods, wherein the cancer in the individual has been determined to further comprise EGFR mutation S310F. In another embodiment is provided such methods, wherein the cancer in the individual has been determined to further comprise EGFR mutation R678Q. In another embodiment is provided such methods, wherein the cancer in the individual has been determined to further comprise EGFR mutation R678Q. In another embodiment is provided such methods, wherein the cancer in the individual has been determined to further comprise EGFR mutation L755W. In another embodiment is provided such methods, wherein the cancer in the individual has been determined to further comprise EGFR mutation L755S. In another embodiment is provided such methods, wherein the cancer in the individual has been determined to further comprise EGFR mutation L755W. In another embodiment is provided such methods, wherein the cancer in the individual has been determined to further comprise EGFR mutation 1767M. In another embodiment is provided such methods, wherein the cancer in the individual has been determined to further comprise EGFR mutation D769H. In another embodiment is provided such methods, wherein the cancer in the individual has been determined to further comprise EGFR mutation D769Y. In another embodiment is provided such methods, wherein the cancer in the individual has been determined to further comprise EGFR mutation V777L. In another embodiment is provided such methods, wherein the cancer in the individual has been determined to further comprise EGFR mutation Y835F. In another embodiment is provided such methods, wherein the cancer in the individual has been determined to further comprise EGFR mutation V842I. In another embodiment is provided such methods, wherein the cancer in the individual has been determined to further comprise EGFR mutation R896C. In another embodiment is provided such methods, wherein the cancer in the individual has been determined to further comprise EGFR mutation G1201V. In another embodiment is provided such methods, wherein the cancer in the individual has been determined to further comprise EGFR mutation EGFR del.755-759. In another embodiment is provided such methods, wherein the cancer in the individual has been determined to further comprise EGFR mutation EGFR Del19/T790M. In another embodiment is provided such methods, wherein the cancer in the individual has been determined to further comprise EGFR mutation L858R/T790M. In another embodiment is provided such methods, wherein the cancer in the individual has been determined to further comprise EGFR mutation EGFR L858R. In another embodiment is provided such methods, wherein the cancer in the individual has been determined to further comprise EGFR mutation EGFR del 19. In another embodiment is provided such methods, wherein the cancer in the individual has been determined to further comprise EGFR mutation EGFR_del746-750. In another embodiment is provided such methods, wherein the cancer in the individual has been determined to further comprise EGFR mutations EGFR L858R/C797S. In another embodiment is provided such methods, wherein the cancer in the individual has been determined to further comprise EGFR mutations EGFR del19/C797S. In another embodiment is provided such methods, wherein the cancer in the individual has been determined to further comprise EGFR mutation EGFR Exon20 ins NPH. In another embodiment is provided such methods, wherein the cancer in the individual has been determined to further comprise EGFR mutation EGFR Exon20 ins SVD. In another embodiment is provided such methods, wherein the cancer in the individual has been determined to further comprise EGFR mutation EGFR Exon20 ins FQEA. In another embodiment is provided such methods, wherein the cancer in the individual has been determined to further comprise EGFR mutation EGFR Exon20 ins H. In another embodiment is provided such methods, wherein the cancer in the individual has been determined to further comprise EGFR mutation EGFR Exon20 ins ASV

In another embodiment is provided such methods, wherein Compound I is administered to the individual in the form of a salt. In another embodiment is provided such methods, wherein Compound I is administered to the individual in the form of a fumarate, hemi-fumarate, glycolate or malonate salt. In another embodiment is provided such methods, wherein Compound I is administered to the individual in the form of a fumarate salt. In another embodiment is provided such methods, wherein Compound I is administered to the individual in the form of a hemi-fumarate salt. In another embodiment is provided such methods, wherein Compound I is administered to the individual in the form of a glycolate salt. In another embodiment is provided such methods, wherein Compound I is administered to the individual in the form of a malonate salt. In another embodiment is provided such methods, wherein Compound I is administered to the individual in the form of a hemi-malonate salt.

In another embodiment is provided such methods, wherein Compound I is administered to the individual in the form of a malonate salt, and wherein the salt is in a crystalline form.

In some embodiments, the crystalline form of the malonate salt maintains at least 95% of the crystalline form following storage in an open container for at least 7 days at 40° C. and 75% relative humidity, and wherein the amount of the crystalline form of the salt is measured by XRPD. In some embodiments, the crystalline form of the malonate salt maintains at least about 95% (e.g., at least 95%, at least 96%, at least 97%, at least 98%, at least 99%, at least 99.25%, at least 99.5%, at least 99.75%, at least 99.8%, or at least 99.9%) of the crystalline form following storage in an open container for at least about 7 days (e.g., 7 days, 8 days, 9 days, 10 days, 11 days, 12 days, 13 days, 2 weeks, one month, two months, three months, 6 months, 12 months, 18 months, or 24 months) at 40° C. and 75% relative humidity, and wherein the amount of the crystalline form of the salt is measured by XRPD. In some embodiments, the crystalline form of the malonate salt maintains at least 95% of the crystalline form following storage in an open container for at least 14 days at 40° C. and 75% relative humidity, and wherein the amount of the crystalline form of the salt is measured by XRPD. In some embodiments, the crystalline form of the malonate salt maintains at least about 95% (e.g., at least 95%, at least 96%, at least 97%, at least 98%, at least 99%, at least 99.25%, at least 99.5%, at least 99.75%, at least 99.8%, or at least 99.9%) of the crystalline form following storage in an open container for at least about 14 days (e.g., 14 days, 15 days, 16 days, 17 days, 18 days, 19 days, 20 days, one month, two months, three months, 6 months, 12 months, 18 months, or 24 months) at 40° C. and 75% relative humidity, and wherein the amount of the crystalline form of the salt is measured by XRPD.

In some embodiments, the crystalline form of the malonate salt maintains at least 95% of the crystalline form following storage in a closed container for at least 7 days at 40° C. and 75% relative humidity, and wherein the amount of the crystalline form of the salt is measured by XRPD. In some embodiments, the crystalline form of the malonate salt maintains at least about 95% (e.g., at least 95%, at least 96%, at least 97%, at least 98%, at least 99%, at least 99.25%, at least 99.5%, at least 99.75%, at least 99.8%, or at least 99.9%) of the crystalline form following storage in a closed container for at least about 7 days (e.g., 7 days, 8 days, 9 days, 10 days, 11 days, 12 days, 13 days, 2 weeks, one month, two months, three months, 6 months, 12 months, 18 months, or 24 months) at 40° C. and 75% relative humidity, and wherein the amount of the crystalline form of the salt is measured by XRPD. In some embodiments, the crystalline form of the malonate salt maintains at least 95% of the crystalline form following storage in a closed container for at least 14 days at 40° C. and 75% relative humidity, and wherein the amount of the crystalline form of the salt is measured by XRPD. In some embodiments, the crystalline form of the malonate salt maintains at least about 95% (e.g., at least 95%, at least 96%, at least 97%, at least 98%, at least 99%, at least 99.25%, at least 99.5%, at least 99.75%, at least 99.8%, or at least 99.9%) of the crystalline form following storage in a closed container for at least about 14 days (e.g., 14 days, 15 days, 16 days, 17 days, 18 days, 19 days, 20 days, one month, two months, three months, 6 months, 12 months, 18 months, or 24 months) at 40° C. and 75% relative humidity, and wherein the amount of the crystalline form of the salt is measured by XRPD.

In some embodiments, the malonate salt has a solubility in an aqueous solution having a pH of about 4.5 and at a temperature of about 37° C., calculated as an amount of the free base of Compound I, of greater than about 2 mg/mL (e.g., greater than about 2.5 mg/mL, greater than about 2.75 mg/mL, greater than about 3 mg/mL, greater than about 3.25 mg/mL, greater than about 3.5 mg/mL, greater than about 3.75 mg/mL, greater than about 4 mg/mL, greater than about 4.25 mg/mL, greater than about 4.5 mg/mL, greater than about 4.75 mg/mL, or greater than about 5 mg/mL). In some embodiments, the malonate salt has a solubility in an aqueous solution having a pH of about 2.9 and at a temperature of about 37° C., calculated as an amount of the free base of Compound I, of greater than about 2 mg/mL (e.g., greater than about 2.5 mg/mL, greater than about 2.75 mg/mL, greater than about 3 mg/mL, greater than about 3.25 mg/mL, greater than about 3.5 mg/mL, greater than about 3.75 mg/mL, greater than about 4 mg/mL, greater than about 4.25 mg/mL, greater than about 4.5 mg/mL, greater than about 4.75 mg/mL, or greater than about 5 mg/mL).

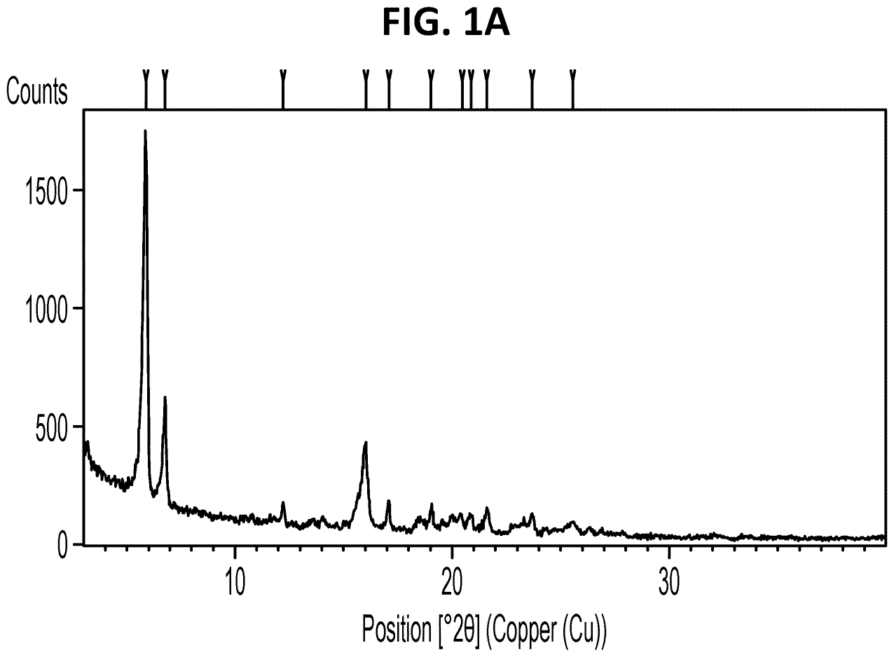

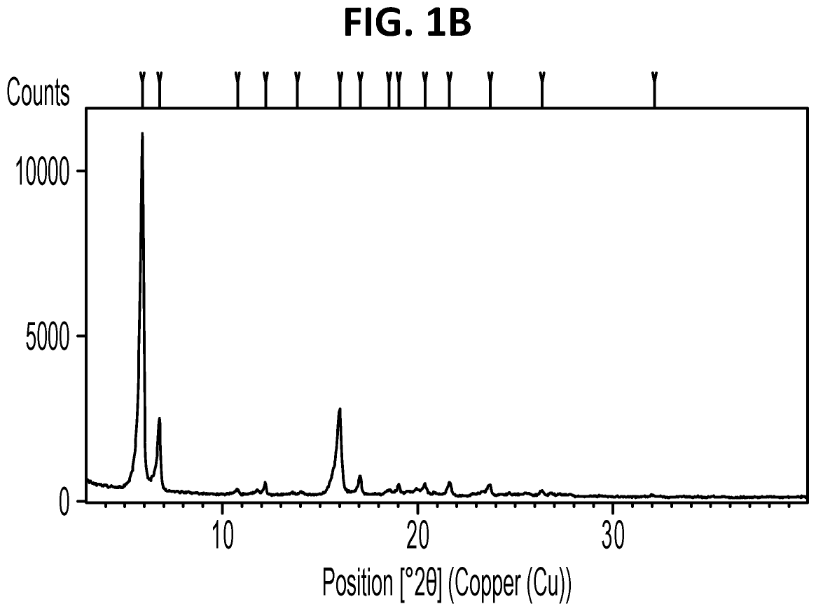

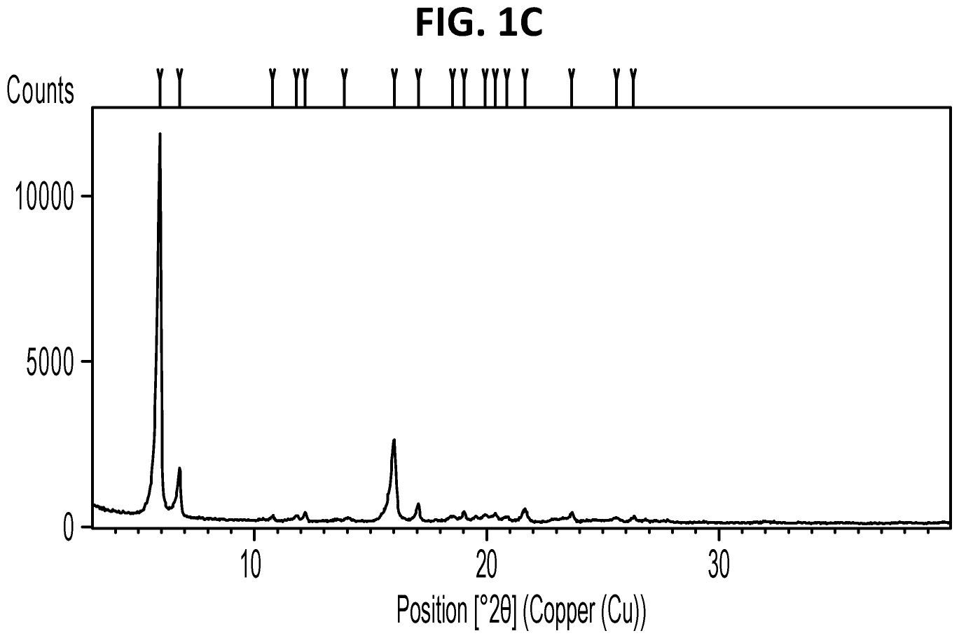

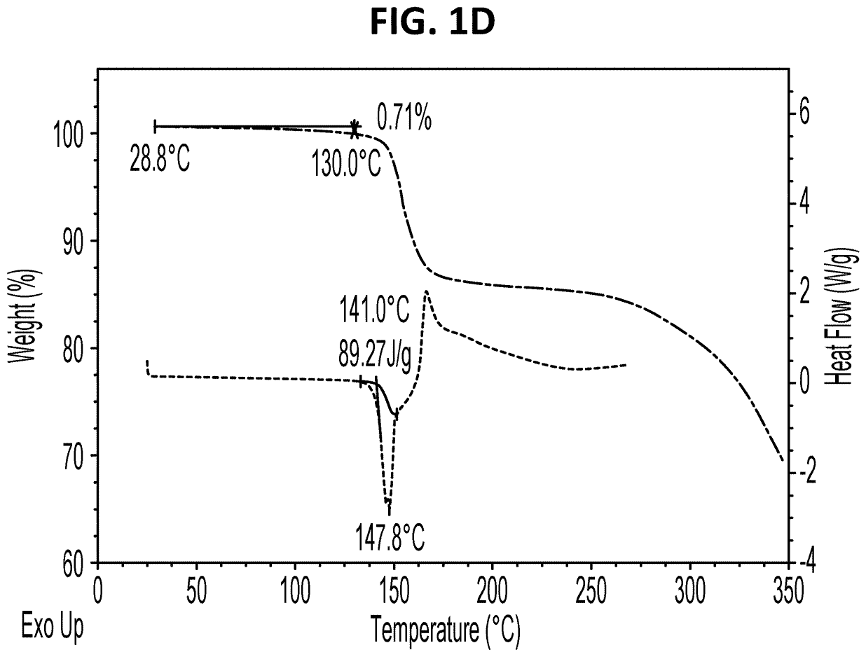

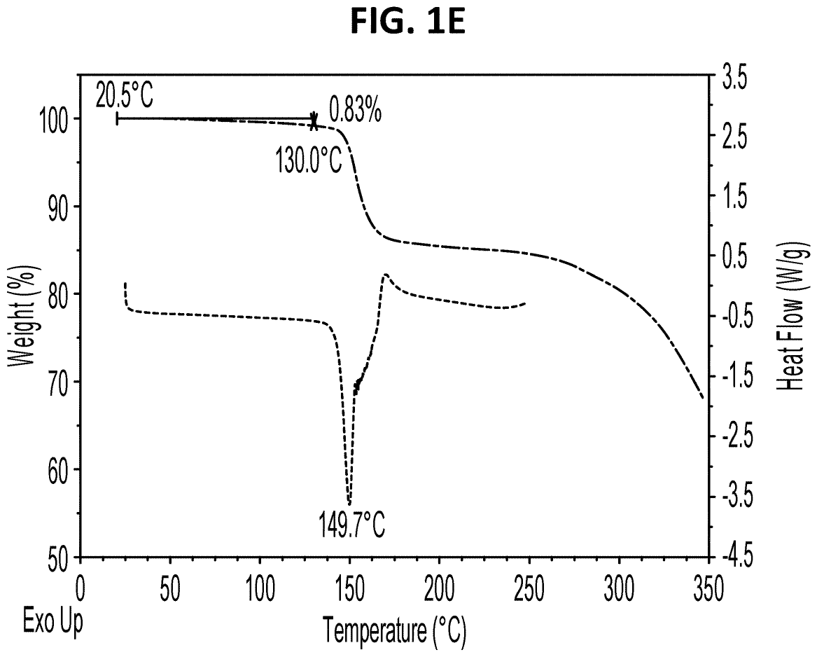

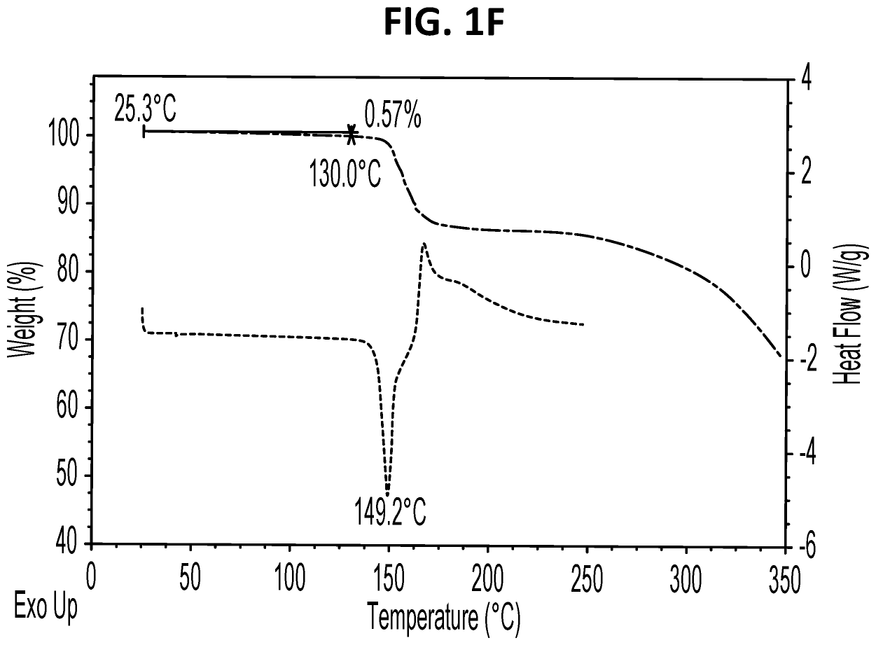

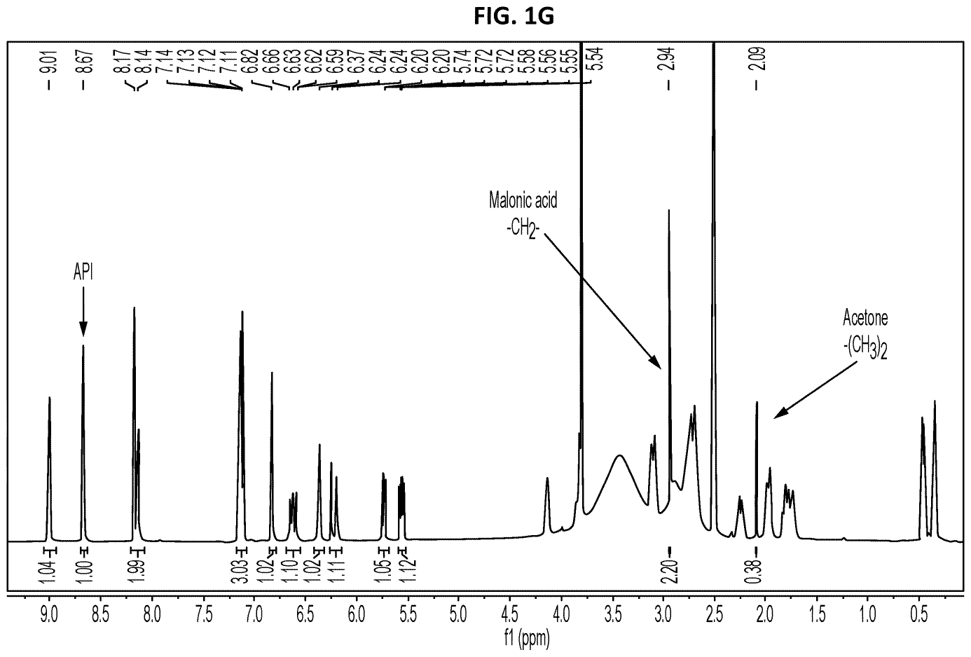

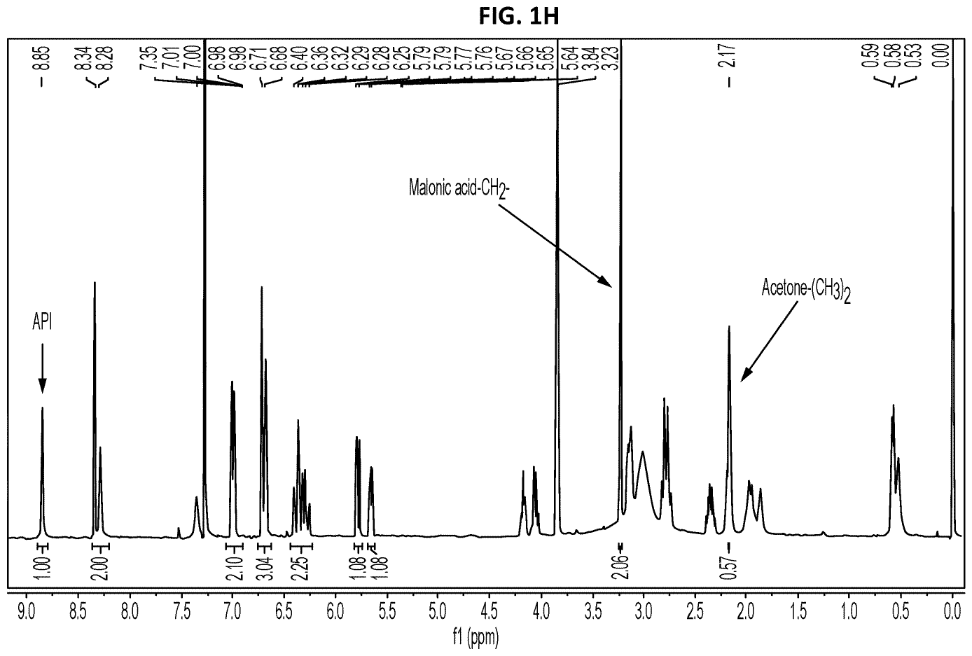

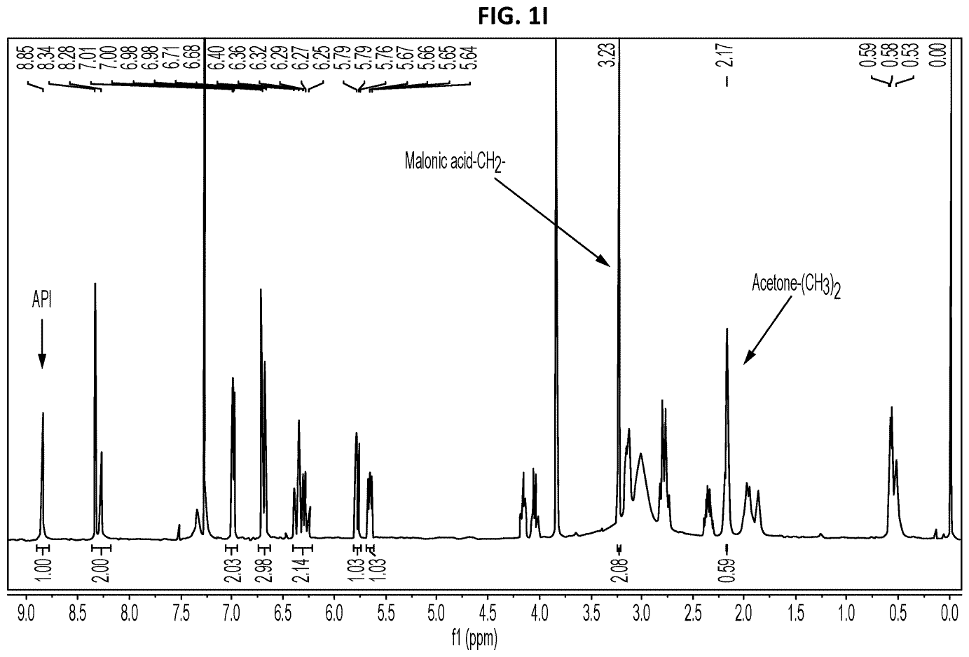

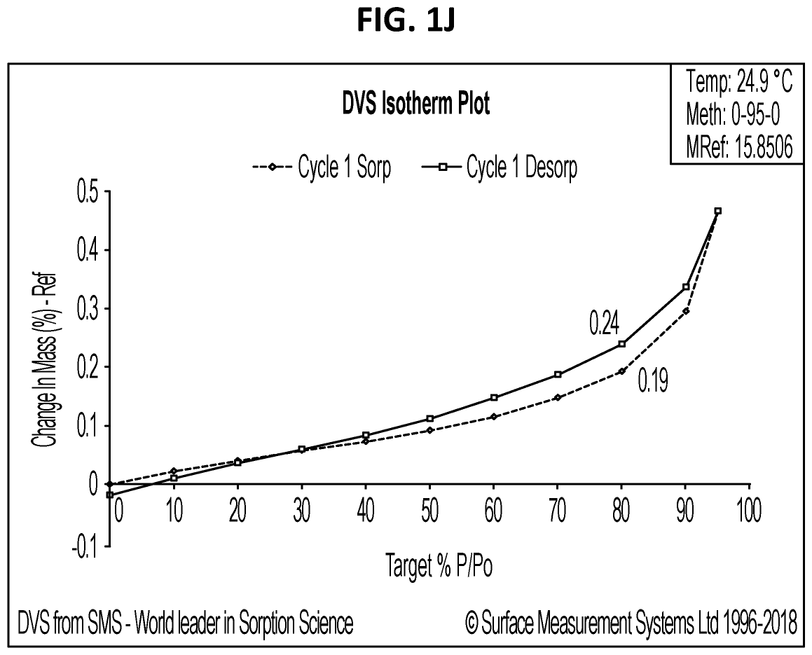

In some embodiments, the malonate salt has an XRPD pattern substantially as shown in FIG. 1A. Angles as measured in degrees 2-theta and peak intensities that may be observed for the malonate salt using XRPD are shown in Table 1.

TABLE 1

| Angle | d value | Intensity |
|---|---|---|
| [°2θ] | (Å) | (counts) |
| 5.9 | 15.0 | 1619.95 |
| 6.8 | 13.0 | 424.53 |
| 12.2 | 7.3 | 83.60 |
| 16.0 | 5.5 | 360.35 |
| 17.1 | 5.2 | 119.80 |
| 19.0 | 4.7 | 93.82 |
| 20.5 | 4.3 | 45.92 |
| 20.9 | 4.3 | 71.21 |
| 21.6 | 4.1 | 97.61 |
| 23.7 | 3.8 | 77.68 |
| 25.6 | 3.5 | 47.76 |

In some embodiments, the malonate salt has an XRPD pattern displaying at least one, at least two, at least three, at least four, at least five, at least six, at least seven, at least eight, at least nine, or at least ten of the peaks at degrees 2-theta with the greatest intensity in the XRPD pattern substantially as shown in FIG. 1A or as provided in Table 1. It should be understood that peak intensities can vary depending on a number of factors, including sample preparation, mounting, and the instrument and analytical procedure and settings used to obtain the spectrum. Peak intensities and peak assignments can vary within experimental error. In some embodiments, peak assignments listed herein, including for the malonate salt, can vary by about ±0.6 degrees, ±0.4 degrees, ±0.2 degrees, or 0.1 degrees 2-theta.

In some embodiments, the malonate salt has an XRPD pattern comprising peaks at 5.9±0.2, 6.8±0.2, 12.2±0.2, 16.0±0.2, 17.1±0.2, 19.0±0.2, 20.5±0.2, 20.9±0.2, 21.6±0.2, 23.7±0.2, and 25.6±0.2 degrees 2-theta. In some embodiments, the malonate salt has an XRPD pattern comprising peaks at 5.9±0.2, 6.8±0.2, 12.2±0.2, 16.0±0.2, 17.1±0.2, 19.0±0.2, 21.6±0.2, and 23.7±0.2 degrees 2-theta. In some embodiments, the malonate salt has an XRPD pattern comprising peaks at 5.9±0.2, 6.8±0.2, 16.0±0.2, and 17.1±0.2 degrees 2-theta. In some embodiments, the malonate salt has an XRPD pattern comprising peaks at 5.9±0.4, 6.8±0.4, 12.2±0.4, 16.0±0.4, 17.1±0.4, 19.0±0.4, 20.5±0.4, 20.9±0.4, 21.6±0.4, 23.7±0.4, and 25.6±0.4 degrees 2-theta. In some embodiments, the malonate salt has an XRPD pattern comprising peaks at 5.9±0.4, 6.8±0.4, 12.2±0.4, 16.0±0.4, 17.1±0.4, 19.0±0.4, 21.6±0.4, and 23.7±0.4 degrees 2-theta. In some embodiments, the malonate salt has an XRPD pattern comprising peaks at 5.9±0.4, 6.8±0.4, 16.0±0.4, and 17.1±0.4 degrees 2-theta. In some embodiments, the malonate salt has an XRPD pattern comprising peaks at 5.9±0.6, 6.8±0.6, 12.2±0.6, 16.0±0.6, 17.1±0.6, 19.0±0.6, 20.5±0.6, 20.9±0.6, 21.6±0.6, 23.7±0.6, and 25.6±0.6 degrees 2-theta. In some embodiments, the malonate salt has an XRPD pattern comprising peaks at 5.9±0.6, 6.8±0.6, 12.2±0.6, 16.0±0.6, 17.1±0.6, 19.0±0.6, 21.6±0.6, and 23.7±0.6 degrees 2-theta. In some embodiments, the malonate salt has an XRPD pattern comprising peaks at 5.9±0.6, 6.8±0.6, 16.0±0.6, and 17.1±0.6 degrees 2-theta. It is to be understood that additional peaks in the XRPD pattern other than those shown in FIG. 1A or as provided in Table 1 may be observed, for instance, due to the presence of impurities, solvent, or other polymorphs or amorphic forms present in the test sample.

In some embodiments, the malonate salt has an XRPD pattern substantially as shown in FIG. 1B. Angles as measured in degrees 2-theta and peak intensities that may be observed for the malonate salt using XRPD are shown in Table 2.

TABLE 2

| Angle | d value | Intensity |
|---|---|---|
| [°2θ] | (Å) | (counts) |
| 5.9 | 15.0 | 11406.7 |
| 6.8 | 13.1 | 2166.3 |
| 10.8 | 8.2 | 142.7 |
| 12.2 | 7.3 | 360.97 |
| 13.9 | 6.4 | 43.57 |
| 16.0 | 5.5 | 2587.22 |
| 17.1 | 5.2 | 587.92 |
| 18.5 | 4.8 | 159.66 |
| 19.0 | 4.7 | 308.38 |
| 20.4 | 4.4 | 368.72 |
| 21.6 | 4.1 | 417.74 |
| 23.7 | 3.8 | 270.03 |
| 26.4 | 3.4 | 107.6 |
| 32.1 | 2.8 | 36.66 |

In some embodiments, the malonate salt has an XRPD pattern displaying at least one, at least two, at least three, at least four, at least five, at least six, at least seven, at least eight, at least nine, or at least ten of the peaks at degrees 2-theta with the greatest intensity in the XRPD pattern substantially as shown in FIG. 1B or as provided in Table 2. It should be understood that peak intensities can vary depending on a number of factors, including sample preparation, mounting, and the instrument and analytical procedure and settings used to obtain the spectrum. Peak intensities and peak assignments can vary within experimental error. In some embodiments, peak assignments listed herein, including for the malonate salt, can vary by about ±0.6 degrees, ±0.4 degrees, ±0.2 degrees, or 0.1 degrees 2-theta.

In some embodiments, the malonate salt has an XRPD pattern comprising peaks at 5.9±0.2, 6.8±0.2, 10.8±0.2, 12.2±0.2, 13.9±0.2, 16.0 0.2, 17.1±0.2, 18.5±0.2, 19.0±0.2, 20.4±0.2, 21.6±0.2, 23.7±0.2, 26.4±0.2, and 32.1±0.2 degrees 2-theta. In some embodiments, the malonate salt has an XRPD pattern comprising peaks at 5.9±0.2, 6.8±0.2, 12.2±0.2, 16.0±0.2, 17.1±0.2, 18.5±0.2, 19.0±0.2, 20.4±0.2, 21.6±0.2, and 23.7±0.2 degrees 2-theta. In some embodiments, the malonate salt has an XRPD pattern comprising peaks at 5.9±0.2, 6.8±0.2, 16.0±0.2, 17.1±0.2, and 21.6±0.2 degrees 2-theta. In some embodiments, the malonate salt has an XRPD pattern comprising peaks at 5.9±0.4, 6.8±0.4, 10.8±0.4, 12.2±0.4, 13.9±0.4, 16.0±0.4, 17.1±0.4, 18.5±0.4, 19.0±0.4, 20.4±0.4, 21.6±0.4, 23.7±0.4, 26.4±0.4, and 32.1±0.4 degrees 2-theta. In some embodiments, the malonate salt has an XRPD pattern comprising peaks at 5.9±0.4, 6.8±0.4, 12.2±0.4, 16.0±0.4, 17.1±0.4, 18.5±0.4, 19.0±0.4, 20.4±0.4, 21.6±0.4, and 23.7±0.4 degrees 2-theta. In some embodiments, the malonate salt has an XRPD pattern comprising peaks at 5.9±0.4, 6.8±0.4, 16.0±0.4, 17.1±0.4, and 21.6±0.4 degrees 2-theta. In some embodiments, the malonate salt has an XRPD pattern comprising peaks at 5.9±0.6, 6.8±0.6, 10.8±0.6, 12.2±0.6, 13.9±0.6, 16.0±0.6, 17.1±0.6, 18.5±0.6, 19.0±0.6, 20.4±0.6, 21.6±0.6, 23.7±0.6, 26.4±0.6, and 32.1±0.6 degrees 2-theta. In some embodiments, the malonate salt has an XRPD pattern comprising peaks at 5.9±0.6, 6.8±0.6, 12.2±0.6, 16.0±0.6, 17.1±0.6, 18.5±0.6, 19.0±0.6, 20.4±0.6, 21.6±0.6, and 23.7±0.6 degrees 2-theta. In some embodiments, the malonate salt has an XRPD pattern comprising peaks at 5.9±0.6, 6.8±0.6, 16.0±0.6, 17.1±0.6, and 21.6±0.6 degrees 2-theta. It is to be understood that additional peaks in the XRPD pattern other than those shown in FIG. 1B or as provided in Table 2 may be observed, for instance, due to the presence of impurities, solvent, or other polymorphs or amorphic forms present in the test sample.

In some embodiments, the malonate salt has an XRPD pattern substantially as shown in FIG. 1C. Angles as measured in degrees 2-theta and peak intensities that may be observed for the malonate salt using XRPD are shown in Table 3.

TABLE 3

| Angle | d value | Intensity |
|---|---|---|
| [°2θ] | (Å) | (counts) |
| 5.9 | 14.9 | 12373.9 |
| 6.8 | 13.1 | 1460.57 |
| 10.8 | 8.2 | 129.02 |
| 11.8 | 7.5 | 141.93 |
| 12.2 | 7.3 | 265.89 |
| 13.9 | 6.4 | 52.99 |
| 16.0 | 5.5 | 2448.17 |
| 17.1 | 5.2 | 524.42 |
| 18.5 | 4.8 | 150.04 |
| 19.0 | 4.7 | 298.32 |
| 19.9 | 4.5 | 200.31 |
| 20.4 | 4.4 | 236.06 |
| 20.9 | 4.3 | 137.62 |
| 21.7 | 4.1 | 384.41 |
| 23.7 | 3.8 | 268.03 |
| 25.6 | 3.5 | 104.88 |
| 26.3 | 3.4 | 114.66 |

In some embodiments, the malonate salt has an XRPD pattern displaying at least one, at least two, at least three, at least four, at least five, at least six, at least seven, at least eight, at least nine, or at least ten of the peaks at degrees 2-theta with the greatest intensity in the XRPD pattern substantially as shown in FIG. 1C or as provided in Table 3. It should be understood that peak intensities can vary depending on a number of factors, including sample preparation, mounting, and the instrument and analytical procedure and settings used to obtain the spectrum. Peak intensities and peak assignments can vary within experimental error. In some embodiments, peak assignments listed herein, including for the malonate salt, can vary by about ±0.6 degrees, ±0.4 degrees, ±0.2 degrees, or ±0.1 degrees 2-theta.

In some embodiments, the malonate salt has an XRPD pattern comprising peaks at 5.9±0.2, 6.8±0.2, 10.8±0.2, 11.8±0.2, 12.2±0.2, 13.9±0.2, 16.0±0.2, 17.1±0.2, 18.5±0.2, 19.0±0.2, 19.9±0.2, 20.4±0.2, 20.9±0.2, 21.7±0.2, 23.7±0.2, 25.6±0.2, and 26.3±0.2 degrees 2-theta. In some embodiments, the malonate salt has an XRPD pattern comprising peaks at 5.9±0.2, 6.8±0.2, 11.8±0.2, 12.2±0.2, 16.0±0.2, 17.1±0.2, 18.5±0.2, 19.0±0.2, 19.9±0.2, 20.4±0.2, 21.7±0.2, and 23.7±0.2 degrees 2-theta. In some embodiments, the malonate salt has an XRPD pattern comprising peaks at 5.9±0.2, 6.8±0.2, 16.0±0.2, 17.1±0.2, 19.0±0.2, 21.7±0.2 degrees 2-theta. In some embodiments, the malonate salt has an XRPD pattern comprising peaks at 5.9±0.4, 6.8±0.4, 10.8±0.4, 11.8±0.4, 12.2±0.4, 13.9±0.4, 16.0±0.4, 17.1±0.4, 18.5±0.4, 19.0±0.4, 19.9±0.4, 20.4±0.4, 20.9±0.4, 21.7±0.4, 23.7±0.4, 25.6±0.4, and 26.3±0.4 degrees 2-theta. In some embodiments, the malonate salt has an XRPD pattern comprising peaks at 5.9±0.4, 6.8±0.4, 11.8±0.4, 12.2±0.4, 16.0±0.4, 17.1±0.4, 18.5±0.4, 19.0±0.4, 19.9±0.4, 20.4±0.4, 21.7±0.4, and 23.7±0.4 degrees 2-theta. In some embodiments, the malonate salt has an XRPD pattern comprising peaks at 5.9±0.4, 6.8±0.4, 16.0±0.4, 17.1±0.4, 19.0±0.4, 21.7±0.4 degrees 2-theta. In some embodiments, the malonate salt has an XRPD pattern comprising peaks at 5.9±0.6, 6.8±0.6, 10.8±0.6, 11.8±0.6, 12.2±0.6, 13.9±0.6, 16.0±0.6, 17.1±0.6, 18.5±0.6, 19.0±0.6, 19.9±0.6, 20.4±0.6, 20.9±0.6, 21.7±0.6, 23.7±0.6, 25.6±0.6, and 26.3±0.6 degrees 2-theta. In some embodiments, the malonate salt has an XRPD pattern comprising peaks at 5.9±0.6, 6.8±0.6, 11.8±0.6, 12.2±0.6, 16.0±0.6, 17.1±0.6, 18.5±0.6, 19.0±0.6, 19.9±0.6, 20.4±0.6, 21.7±0.6, and 23.7±0.6 degrees 2-theta. In some embodiments, the malonate salt has an XRPD pattern comprising peaks at 5.9±0.6, 6.8±0.6, 16.0±0.6, 17.1±0.6, 19.0±0.6, 21.7±0.6 degrees 2-theta. It is to be understood that additional peaks in the XRPD pattern other than those shown in FIG. 1C or as provided in Table 3 may be observed, for instance, due to the presence of impurities, solvent, or other polymorphs or amorphic forms present in the test sample.

In some embodiments, the malonate salt has an XRPD pattern comprising a peak at 5.9±0.2 degrees 2-theta. In some embodiments, the malonate salt has an XRPD pattern comprising peaks at 5.9±0.2 and 6.8±0.2 degrees 2-theta. In some embodiments, the malonate salt has an XRPD pattern comprising peaks at 5.9±0.2, 6.8±0.2, and 16.0±0.2 degrees 2-theta. In some embodiments, the malonate salt has an XRPD pattern comprising peaks at 5.9±0.2, 6.8±0.2, 16.0±0.2, 17.1±0.2, 19.0±0.2, and 21.6±0.2 degrees 2-theta. In some embodiments, the malonate salt has an XRPD pattern comprising peaks at 5.9±0.2, 6.8±0.2, 12.2±0.2, 16.0±0.2, 17.1±0.2, 19.0±0.2, 20.5±0.2, 21.6±0.2, and 23.7±0.2 degrees 2-theta. In some embodiments, the malonate salt has an XRPD pattern comprising a peak at 5.9±0.4 degrees 2-theta. In some embodiments, the malonate salt has an XRPD pattern comprising peaks at 5.9±0.4 and 6.8±0.4 degrees 2-theta. In some embodiments, the malonate salt has an XRPD pattern comprising peaks at 5.9±0.4, 6.8±0.4, and 16.0±0.4 degrees 2-theta. In some embodiments, the malonate salt has an XRPD pattern comprising peaks at 5.9±0.4, 6.8±0.4, 16.0±0.4, 17.1±0.4, 19.0±0.4, and 21.6±0.4 degrees 2-theta. In some embodiments, the malonate salt has an XRPD pattern comprising peaks at 5.9±0.4, 6.8±0.4, 12.2±0.4, 16.0±0.4, 17.1±0.4, 19.0±0.4, 20.5±0.4, 21.6±0.4, and 23.7±0.4 degrees 2-theta. In some embodiments, the malonate salt has an XRPD pattern comprising a peak at 5.9±0.6 degrees 2-theta. In some embodiments, the malonate salt has an XRPD pattern comprising peaks at 5.9±0.6 and 6.8±0.6 degrees 2-theta. In some embodiments, the malonate salt has an XRPD pattern comprising peaks at 5.9±0.6, 6.8±0.6, and 16.0±0.6 degrees 2-theta. In some embodiments, the malonate salt has an XRPD pattern comprising peaks at 5.9±0.6, 6.8±0.6, 16.0±0.6, 17.1±0.6, 19.0±0.6, and 21.6±0.6 degrees 2-theta. In some embodiments, the malonate salt has an XRPD pattern comprising peaks at 5.9±0.6, 6.8±0.6, 12.2±0.6, 16.0±0.6, 17.1±0.6, 19.0±0.6, 20.5±0.6, 21.6±0.6, and 23.7±0.6 degrees 2-theta.

In some embodiments, the malonate salt has a differential scanning calorimetry trace substantially as shown in FIG. 1D. In some embodiments, the malonate salt has a differential scanning calorimetry trace substantially as shown in FIG. 1E. In some embodiments, the malonate salt has a differential scanning calorimetry trace substantially as shown in FIG. 1F. In some embodiments, the malonate salt is characterized as exhibiting a differential scanning calorimetry trace comprising a peak of from about 147° C. to about 151° C. In some embodiments, the malonate salt is characterized as exhibiting a differential scanning calorimetry trace comprising a peak at 147±4° C. (e.g., 147±3° C., 147±2° C., or 147±1° C.). In some embodiments, the malonate salt is characterized as exhibiting a differential scanning calorimetry trace comprising a peak at 149±4° C. (e.g., 149±3° C., 149±2° C., or 149±1° C.). In some embodiments, the malonate salt is characterized as exhibiting a differential scanning calorimetry trace comprising a peak at 151±4° C. (e.g., 151±3° C., 151±2° C., or 1511° C.). In some embodiments, the malonate salt is characterized as exhibiting a differential scanning calorimetry trace comprising a peak of from about 147° C. to about 150° C. In some embodiments, the malonate salt is characterized as exhibiting a differential scanning calorimetry trace comprising a peak of from about 148° C. to about 151° C. In some embodiments, the malonate salt is characterized as exhibiting a differential scanning calorimetry trace comprising a peak of from about 148° C. to about 150° C. In some embodiments, the malonate salt is characterized as exhibiting a differential scanning calorimetry trace comprising a peak of from about 147° C. to about 149° C. In some embodiments, the malonate salt is characterized as exhibiting a differential scanning calorimetry trace comprising a peak of from about 149° C. to about 151° C.

In some embodiments of the malonate salt, at least one, at least two, at least three, at least four, at least five, at least six, at least seven, or all of the following (a)-(h) apply:
(a) the malonate salt is a hemi-malonate salt;
(b) the malonate salt is in a crystalline form;
(c) the crystalline form of the malonate salt maintains at least 95% of the crystalline form following storage in an open container for at least 7 days at 40° C. and 75% relative humidity, and wherein the amount of the crystalline form of the salt is measured by XRPD,
(d) the crystalline form of the malonate salt maintains at least 95% of the crystalline form following storage in a closed container for at least 7 days at 40° C. and 75% relative humidity, and wherein the amount of the crystalline form of the salt is measured by XRPD,
(e) the malonate salt has a solubility in an aqueous solution having a pH of about 4.5 and at a temperature of about 37° C., calculated as an amount of the free base of Compound I, of greater than about 2 mg/mL;
(f) the malonate salt has a solubility in an aqueous solution having a pH of about 2.9 and at a temperature of about 37° C., calculated as an amount of the free base of Compound I, of greater than about 2 mg/mL;
(g) the malonate salt exhibits an XRPD pattern comprising:
   (i) a peak at 5.9±0.2 degrees 2-theta;
   (ii) peaks at 5.9±0.2 and 6.8±0.2 degrees 2-theta;
   (iii) peaks at 5.9±0.2, 6.8±0.2, and 16.0±0.2 degrees 2-theta;
   (iv) peaks at 5.9±0.2, 6.8±0.2, 16.0±0.2, 17.1±0.2, 19.0±0.2, and 21.6±0.2 degrees 2-theta; or
   (v) peaks at 5.9±0.2, 6.8±0.2, 12.2±0.2, 16.0±0.2, 17.1±0.2, 19.0±0.2, 20.5±0.2, 21.6±0.2, and 23.7±0.2 degrees 2-theta; and
   (h) the malonate salt exhibits a differential scanning calorimetry trace comprising a peak of from about 147° C. to about 151° C.

In some embodiments, (a) and (b) apply. In some embodiments, (a), (b), and (c) apply. In some embodiments, (a), (b), (c), and (d) apply. In some embodiments, (a), (b), (c), (d), and (e) apply. In some embodiments, (a), (b), (c), (d), (e), and (f) apply. In some embodiments, (a), (b), (c), (d), (e), (f), and (g) apply. In some embodiments, (a), (b), (c), (d), (e), (f), (g), and (h) apply. In some embodiments, (g) and (h) apply. In some embodiments, (g)(i) and (h) apply. In some embodiments, (g)(ii) and (h) apply. In some embodiments, (g)(iii) and (h) apply. In some embodiments, (g)(iv) and (h) apply. In some embodiments, (g)(v) and (h) apply. In some embodiments, (e), (g), and (h) apply. In some embodiments, (e), (g)(i), and (h) apply. In some embodiments, (e), (g)(ii), and (h) apply. In some embodiments, (e), (g)(iii), and (h) apply. In some embodiments, (e), (g)(iv), and (h) apply. In some embodiments, (e), (g)(v), and (h) apply. In some embodiments, (f), (g), and (h) apply. In some embodiments, (f), (g)(i), and (h) apply. In some embodiments, (f), (g)(ii), and (h) apply. In some embodiments, (f), (g)(iii), and (h) apply. In some embodiments, (f), (g)(iv), and (h) apply. In some embodiments, (f), (g)(v), and (h) apply. In some embodiments, (e), (f), (g), and (h) apply. In some embodiments, (e), (f), (g)(i), and (h) apply. In some embodiments, (e), (f), (g)(ii), and (h) apply. In some embodiments, (e), (f), (g)(iii), and (h) apply. In some embodiments, (e), (f), (g)(iv), and (h) apply. In some embodiments, (e), (f), (g)(v), and (h) apply.

### Glycolate Salt

In some embodiments, are provided methods of treating cancer in an individual in need thereof, wherein in the cancer in the individual has been determined to comprise one or more atypical epidermal growth factor receptor (EGFR) mutations, comprising administering to the individual Compound I in the form of a glycolate salt. In some embodiments, the glycolate salt of Compound I is a hemi-glycolate salt.

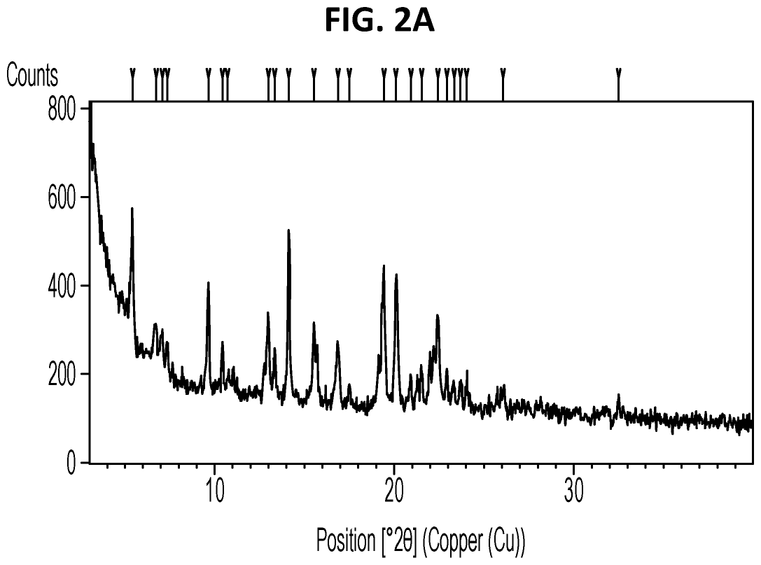

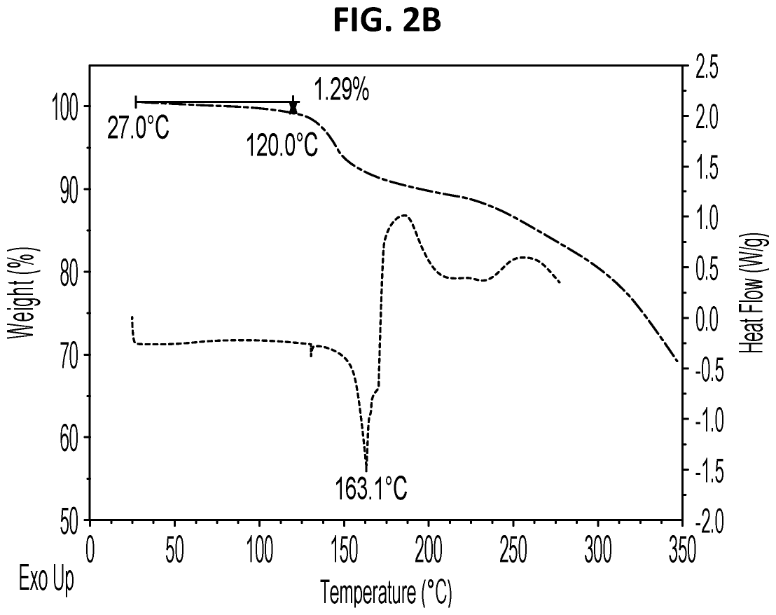

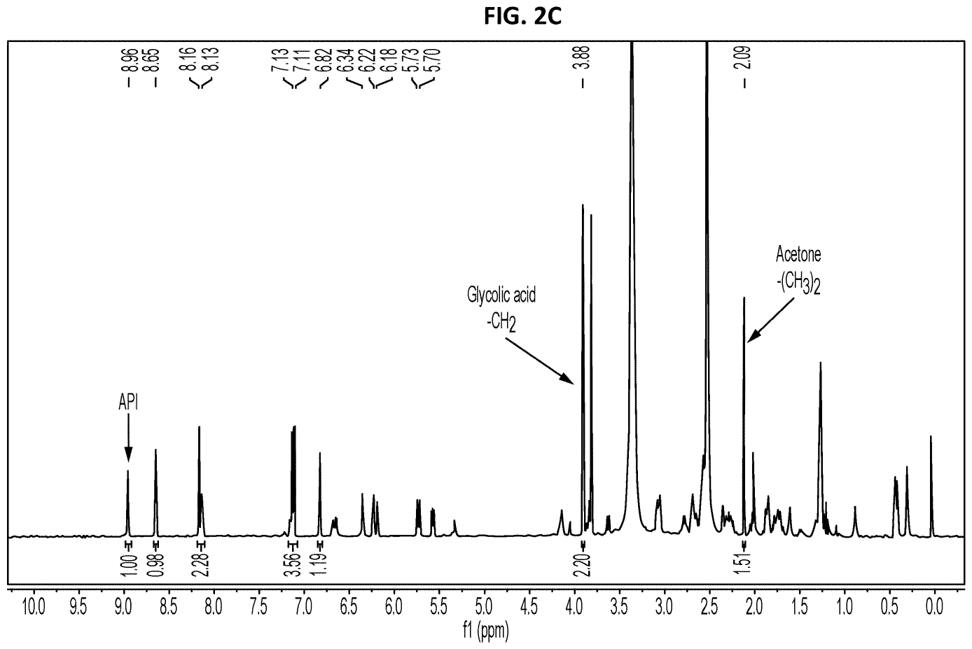

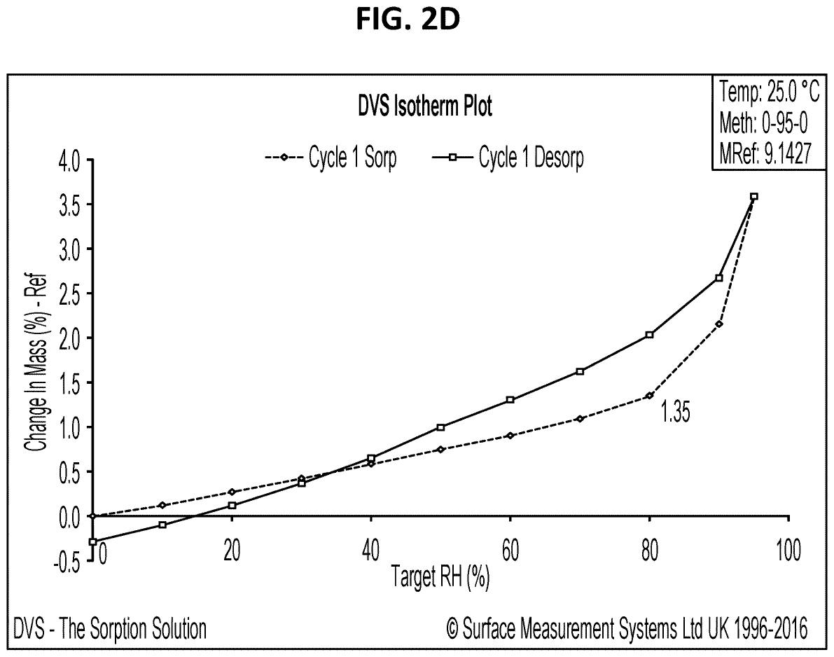

In some embodiments, the glycolate salt of Compound I is in a crystalline form. In some embodiments, the glycolate salt has an XRPD pattern substantially as shown in FIG. 2A. Angles as measured in degrees 2-theta and peak intensities that may be observed for the glycolate salt using XRPD are shown in Table 4.

TABLE 4

| Angle | d value | Intensity |
|---|---|---|
| [°2θ] | (Å) | (counts) |
| 5.4 | 16.3 | 271.16 |
| 6.7 | 13.1 | 85.19 |
| 7.1 | 12.5 | 90.56 |
| 7.4 | 12.0 | 66.16 |
| 9.6 | 9.2 | 240.58 |
| 10.4 | 8.5 | 109.09 |
| 10.7 | 8.3 | 21.56 |
| 13.0 | 6.8 | 192.16 |
| 13.3 | 6.6 | 110.32 |
| 14.1 | 6.3 | 367.49 |
| 15.5 | 5.7 | 179.23 |
| 16.9 | 5.3 | 143.13 |
| 17.5 | 5.1 | 42.23 |
| 19.4 | 4.6 | 311.96 |
| 20.1 | 4.4 | 282.66 |
| 20.9 | 4.3 | 53.9 |
| 21.5 | 4.1 | 61.7 |
| 22.4 | 4.0 | 188.55 |
| 22.9 | 3.9 | 62.88 |
| 23.3 | 3.8 | 23.48 |
| 23.7 | 3.8 | 27.46 |
| 24.0 | 3.7 | 41.88 |
| 26.1 | 3.4 | 24.09 |
| 32.5 | 2.8 | 49.51 |

In some embodiments, the glycolate salt has an XRPD pattern displaying at least one, at least two, at least three, at least four, at least five, at least six, at least seven, at least eight, at least nine, or at least ten of the peaks at degrees 2-theta with the greatest intensity in the XRPD pattern substantially as shown in FIG. 2A or as provided in Table 4. It should be understood that peak intensifies can vary depending on a number of factors, including sample preparation, mounting, and the instrument and analytical procedure and settings used to obtain the spectrum. Peak intensities and peak assignments can vary within experimental error. In some embodiments, peak assignments listed herein, including for the glycolate salt, can vary by about ±0.6 degrees, ±0.4 degrees, ±0.2 degrees, or 0.1 degrees 2-theta.

In some embodiments, the glycolate salt has an XRPD pattern comprising peaks at 5.4±0.2, 6.7±0.2, 7.1±0.2, 7.4±0.2, 9.6±0.2, 10.4±0.2, 10.7±0.2, 13.0±0.2, 13.3±0.2, 14.1±0.2, 15.5±0.2, 16.9±0.2, 17.5±0.2, 19.4±0.2, 20.1±0.2, 20.9±0.2, 21.5±0.2, 22.4±0.2, 22.9±0.2, 23.3±0.2, 23.7±0.2, 24±0.2, 26.1±0.2, and 32.5±0.2 degrees 2-theta. In some embodiments, the glycolate salt has an XRPD pattern comprising peaks at 5.4±0.2, 6.7±0.2, 7.1±0.2, 7.4±0.2, 9.6±0.2, 10.4±0.2, 13.0±0.2, 13.3±0.2, 14.1±0.2, 15.5±0.2, 16.9±0.2, 19.4±0.2, 20.1±0.2, 21.5±0.2, 22.4±0.2, and 22.9±0.2 degrees 2-theta. In some embodiments, the glycolate salt has an XRPD pattern comprising peaks at 5.4±0.2, 9.6±0.2, 13.0±0.2, 14.1±0.2, 15.5±0.2, 19.4±0.2, 20.1±0.2, and 22.4±0.2 degrees 2-theta. In some embodiments, the glycolate salt has an XRPD pattern comprising peaks at 5.4±0.4, 6.7±0.4, 7.1±0.4, 7.4±0.4, 9.6±0.4, 10.4±0.4, 10.7±0.4, 13.0±0.4, 13.3±0.4, 14.1±0.4, 15.5±0.4, 16.9±0.4, 17.5±0.4, 19.4±0.4, 20.1±0.4, 20.9±0.4, 21.5±0.4, 22.4±0.4, 22.9±0.4, 23.3±0.4, 23.7±0.4, 24±0.4, 26.1±0.4, and 32.5±0.4 degrees 2-theta. In some embodiments, the glycolate salt has an XRPD pattern comprising peaks at 5.4±0.4, 6.7±0.4, 7.1±0.4, 7.4±0.4, 9.6±0.4, 10.4±0.4, 13.0 0.4, 13.3±0.4, 14.1±0.4, 15.5±0.4, 16.9±0.4, 19.4±0.4, 20.1±0.4, 21.5±0.4, 22.4±0.4, and 22.9±0.4 degrees 2-theta. In some embodiments, the glycolate salt has an XRPD pattern comprising peaks at 5.4±0.4, 9.6±0.4, 13.0±0.4, 14.1±0.4, 15.5±0.4, 19.4±0.4, 20.1±0.4, and 22.4±0.4 degrees 2-theta. In some embodiments, the glycolate salt has an XRPD pattern comprising peaks at 5.4±0.6, 6.7±0.6, 7.1±0.6, 7.4±0.6, 9.6±0.6, 10.4±0.6, 10.7±0.6, 13.0±0.6, 13.3±0.6, 14.1±0.6, 15.5±0.6, 16.9±0.6, 17.5±0.6, 19.4±0.6, 20.1±0.6, 20.9±0.6, 21.5±0.6, 22.4±0.6, 22.9±0.6, 23.3±0.6, 23.7±0.6, 24±0.6, 26.1±0.6, and 32.5±0.6 degrees 2-theta. In some embodiments, the glycolate salt has an XRPD pattern comprising peaks at 5.4±0.6, 6.7±0.6, 7.1±0.6, 7.4±0.6, 9.6±0.6, 10.4±0.6, 13.0±0.6, 13.3±0.6, 14.1±0.6, 15.5±0.6, 16.9±0.6, 19.4±0.6, 20.1±0.6, 21.5±0.6, 22.4±0.6, and 22.9±0.6 degrees 2-theta. In some embodiments, the glycolate salt has an XRPD pattern comprising peaks at 5.4±0.6, 9.6±0.6, 13.0±0.6, 14.1±0.6, 15.5±0.6, 19.4±0.6, 20.1±0.6, and 22.4±0.6 degrees 2-theta. It is to be understood that additional peaks in the XRPD pattern other than those shown in FIG. 2A or as provided in Table 4 may be observed, for instance, due to the presence of impurities, solvent, or other polymorphs or amorphic forms present in the test sample.

In some embodiments, the glycolate salt has an XRPD pattern comprising a peak at 14.1±0.2 degrees 2-theta. In some embodiments, the glycolate salt has an XRPD pattern comprising peaks at 14.1±0.2 and 19.4±0.2 degrees 2-theta. In some embodiments, the glycolate salt has an XRPD pattern comprising peaks at 14.1±0.2, 19.4±0.2, and 20.1±0.2 degrees 2-theta. In some embodiments, the glycolate salt has an XRPD pattern comprising peaks at 5.4±0.2, 9.6±0.2, 13.0±0.2, 14.1±0.2, 15.5±0.2, 19.4±0.2, 20.1±0.2, and 22.4±0.2 degrees 2-theta. In some embodiments, the glycolate salt has an XRPD pattern comprising a peak at 14.1±0.4 degrees 2-theta. In some embodiments, the glycolate salt has an XRPD pattern comprising peaks at 14.1±0.4 and 19.4±0.4 degrees 2-theta. In some embodiments, the glycolate salt has an XRPD pattern comprising peaks at 14.1±0.4, 19.4±0.4, and 20.1±0.4 degrees 2-theta. In some embodiments, the glycolate salt has an XRPD pattern comprising peaks at 5.4±0.4, 9.6±0.4, 13.0±0.4, 14.1±0.4, 15.5±0.4, 19.4±0.4, 20.1±0.4, and 22.4±0.4 degrees 2-theta. In some embodiments, the glycolate salt has an XRPD pattern comprising a peak at 14.1±0.6 degrees 2-theta. In some embodiments, the glycolate salt has an XRPD pattern comprising peaks at 14.1±0.6 and 19.4±0.6 degrees 2-theta. In some embodiments, the glycolate salt has an XRPD pattern comprising peaks at 14.1±0.6, 19.4±0.6, and 20.1±0.6 degrees 2-theta. In some embodiments, the glycolate salt has an XRPD pattern comprising peaks at 5.4±0.6, 9.6±0.6, 13.0±0.6, 14.1±0.6, 15.5±0.6, 19.4±0.6, 20.1±0.6, and 22.4±0.6 degrees 2-theta.

In some embodiments, the glycolate salt has a differential scanning calorimetry trace substantially as shown in FIG. 2B. In some embodiments, the glycolate salt is characterized as exhibiting a differential scanning calorimetry trace comprising a peak of from about 162° C. to about 165° C. In some embodiments, the glycolate salt is characterized as exhibiting a differential scanning calorimetry trace comprising a peak at 162±4° C. (e.g., 162±3° C., 162±2° C., or 162±1° C.). In some embodiments, the glycolate salt is characterized as exhibiting a differential scanning calorimetry trace comprising a peak at 163±4° C. (e.g., 163±3° C., 163±2° C., or 163±1° C.). In some embodiments, the glycolate salt is characterized as exhibiting a differential scanning calorimetry trace comprising a peak at 165±4° C. (e.g., 165±3° C., 165±2° C., or 165±1° C.). In some embodiments, the glycolate salt is characterized as exhibiting a differential scanning calorimetry trace comprising a peak of from about 162° C. to about 164° C. In some embodiments, the glycolate salt is characterized as exhibiting a differential scanning calorimetry trace comprising a peak of from about 163° C. to about 165° C.

In some embodiments of the glycolate salt, at least one, at least two, at least three, or all of the following (a)-(d) apply:
(a) the glycolate salt is a hemi-glycolate salt;
(b) the glycolate salt is in a crystalline form;
(c) the glycolate salt exhibits an XRPD pattern comprising:
   (i) a peak at 14.1±0.2 degrees 2-theta;
   (ii) peaks at 14.1±0.2 and 19.4±0.2 degrees 2-theta;
   (iii) peaks at 14.1±0.2, 19.4±0.2, and 20.1±0.2 degrees 2-theta; or
   (iv) peaks at 5.4±0.2, 9.6±0.2, 13.0±0.2, 14.1±0.2, 15.5±0.2, 19.4±0.2, 20.1±0.2, and 22.4±0.2 degrees 2-theta; and
(d) the glycolate salt exhibits a differential scanning calorimetry trace comprising a peak of from about 162° C. to about 165° C.

In some embodiments, (a) and (b) apply. In some embodiments, (a), (b), and (c) apply. In some embodiments, (a), (b), (c), and (d) apply. In some embodiments, (c) and (d) apply. In some embodiments, (cxi) and (d) apply. In some embodiments, (cxii) and (d) apply. In some embodiments, (cxiii) and (d) apply. In some embodiments, (cxiv) and (d) apply.

### Compositions

Also provided herein are methods of treating cancer in an individual in need thereof, wherein in the cancer in the individual has been determined to comprise one or more atypical epidermal growth factor receptor (EGFR) mutations, comprising administering to the individual Compound I, or a pharmaceutically acceptable salt thereof, in the form of a pharmaceutically acceptable composition. Such pharmaceutically acceptable compositions comprise Compound I, or a pharmaceutically acceptable salt thereof, and one or more pharmaceutically acceptable carrier. In another embodiment is provided such methods, wherein the pharmaceutically acceptable composition comprises Compound I in the form of a fumarate, hemi-fumarate, glycolate or malonate salt. In another embodiment is provided such methods, wherein the pharmaceutically acceptable composition comprises Compound I in the form of a fumarate salt. In another embodiment is provided such methods, wherein the pharmaceutically acceptable composition comprises Compound I in the form of a hemi-fumarate salt. In another embodiment is provided such methods, wherein the pharmaceutically acceptable composition comprises Compound I in the form of a glycolate salt. In another embodiment is provided such methods, wherein Compound I is administered to the individual in the form of a malonate salt. In another embodiment is provided such methods, wherein the pharmaceutically acceptable composition comprises Compound I in the form of a hemi-malonate salt. In some embodiments, the composition is a sterile composition. In some embodiments, the pharmaceutical composition comprising Compound I, or a pharmaceutically acceptable salt thereof, and one or more pharmaceutically acceptable carriers is substantially free of other salts and non-salt forms of Compound I. In some embodiments, the composition is substantially free of amorphous or non-crystalline forms of Compound I.

In some embodiments of the composition containing a malonate salt of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is the malonate salt. In some embodiments of the composition containing a malonate salt of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I in the composition exists as the malonate salt.

In some embodiments of the composition containing a glycolate salt of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is the glycolate salt. In some embodiments of the composition containing a glycolate salt of Compound I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound I in the composition exists as the glycolate salt.

Also provided herein is the use of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, in the manufacture of a medicament for treatment of cancer in an individual in need thereof, wherein in the cancer in the individual has been determined to comprise one or more atypical epidermal growth factor receptor (EGFR) mutations as described herein.

Also provided herein is the use of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, for the treatment of cancer in an individual in need thereof, wherein in the cancer in the individual has been determined to comprise one or more atypical epidermal growth factor receptor (EGFR) mutations as described herein.

Also provided herein is (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, for the treatment of cancer in an individual in need thereof, wherein in the cancer in the individual has been determined to comprise one or more atypical epidermal growth factor receptor (EGFR) mutations as described herein.

Also provided herein is (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, for use as a medicament for use in the treatment of cancer in an individual in need thereof, wherein in the cancer in the individual has been determined to comprise one or more atypical epidermal growth factor receptor (EGFR) mutations as described herein.

In some embodiments of the methods provided herein, the cancer in the individual is selected from the group consisting of pseudomyxoma, intrahepatic cholangiocarcinoma, hepatoblastoma, liver cancer, thyroid cancer, colon cancer, testicular cancer, myelodysplastic syndrome, glioblastoma, oral cancer, lip cancer, mycosis fungoides, acute myeloid leukemia, acute lymphocytic leukemia, basal cell carcinoma, ovarian epithelial cancer, ovarian germ cell cancer, male breast cancer, brain cancer, pituitary adenomas, multiple myeloma, gallbladder cancer, biliary tract cancer, colorectal cancer, chronic myelogenous leukemia, chronic lymphocytic leukemia, retinoblastoma, choroidal melanoma, ampulla of vater cancer, bladder cancer, peritoneal cancer, parathyroid cancer, adrenal cancer, nasal and paranasal sinus cancer, non-small cell lung cancer, tongue cancer, astrocytoma, small cell lung cancer, childhood brain cancer, pediatric lymphoma, pediatric leukemia, small intestine cancer, meningioma, esophageal cancer, glioma, renal carcinoma, kidney cancer, heart cancer, duodenal cancer, malignant soft tissue cancer, malignant bone cancer, malignant lymphoma, malignant mesothelioma, malignant melanoma, eye cancer, vulvar cancer, ureter cancer, urethral cancer, cancer of unknown primary site, gastric lymphoma, gastric cancer, gastric carcinoid tumors, gastrointestinal stromal tumors, Wilms cancer, breast cancer, sarcoma, penile cancer, pharyngeal cancer, gestational trophoblastic disease, cervical cancer, endometrial cancer, uterine sarcoma, prostate cancer, metastatic bone cancer, metastatic brain cancer, mediastinal cancer, rectal cancer, rectal carcinoid tumors, vaginal cancer, spinal carcinoma, acoustic neuroma, pancreatic cancer, salivary gland cancer, Kaposi's sarcoma, Paget's disease, tonsillar cancer, squamous cell carcinoma, lung adenocarcinoma, lung cancer, lung squamous cell carcinoma, skin cancer, anal cancer, rhabdomyosarcoma, laryngeal cancer, pleural cancer, blood cancer, and thymic carcinoma. In some embodiments, the cancer is selected from the group consisting of metastatic brain cancer, breast cancer, and non-small cell lung cancer. In some embodiments, the cancer is metastatic brain cancer. In some embodiments, the cancer is breast cancer. In some embodiments, the cancer is non-small cell lung cancer. In some variations of any of the embodiments described herein, the cancer is a locally advanced cancer. In some variations, the cancer is unresectable. In some variations, the cancer comprises one or more central nervous system (CNS) metastases (e.g., brain metastases).

In some embodiments of the methods disclosed herein, the individual has not received one or more prior therapy for treatment of the cancer prior to administration to the individual of Compound I, or a pharmaceutically acceptable salt thereof. In some embodiments, the individual has not received one or more (such as 1, 2, or 3) prior therapy for treatment of the cancer prior to administration to the individual of a salt of Compound I as described herein or a pharmaceutical composition comprising a salt of Compound I as described herein.

In some embodiments of the methods disclosed herein, the cancer is non-small cell lung cancer and the individual has not received prior therapy for treatment of the cancer with an EGFR inhibitor prior to administration to the individual of Compound I, or a pharmaceutically acceptable salt thereof. In some embodiments, the individual has not received prior therapy for treatment of the cancer with an EGFR inhibitor prior to administration to the individual of a salt of Compound I as described herein or a pharmaceutical composition comprising a salt of Compound I as described herein. In some embodiments, the cancer has been determined to comprise one or more atypical epidermal growth factor receptor (EGFR) mutations, and the individual has not received prior therapy for treatment of the cancer with an EGFR inhibitor prior to administration to the individual of a salt of Compound I as described herein or a pharmaceutical composition comprising a salt of Compound I as described herein. In some embodiments, the cancer is non-small cell lung cancer that has been determined to comprise one or more atypical epidermal growth factor receptor (EGFR) mutations, and the individual has not received prior therapy for treatment of the cancer with an EGFR inhibitor prior to administration to the individual of a salt of Compound I as described herein or a pharmaceutical composition comprising a salt of Compound I as described herein. In some embodiments, the cancer is metastatic non-small cell lung cancer that has been determined to comprise one or more atypical epidermal growth factor receptor (EGFR) mutations, and the individual has not received prior therapy for treatment of the cancer with an EGFR inhibitor prior to administration to the individual of a salt of Compound I as described herein or a pharmaceutical composition comprising a salt of Compound I as described herein. In some embodiments, the cancer is non-small cell lung cancer comprising central nervous system (CNS) metastases (e.g., brain metastases) that has been determined to comprise one or more atypical epidermal growth factor receptor (EGFR) mutations, and the individual has not received prior therapy for treatment of the cancer with an EGFR inhibitor prior to administration to the individual of a salt of Compound I as described herein or a pharmaceutical composition comprising a salt of Compound I as described herein In some embodiments, the salt of Compound I is the malonate salt. In some embodiments, the pharmaceutical composition comprises the malonate salt of Compound I.

In some embodiments of the methods disclosed herein, the individual is EGFR inhibitor naïve prior to administration to the individual of Compound I, or a pharmaceutically acceptable salt thereof. In some embodiments, the cancer is non-small cell lung cancer. In some embodiments, the cancer has been determined to comprise one or more atypical epidermal growth factor receptor (EGFR) mutations, and the individual is EGFR inhibitor naïve prior to administration to the individual of a salt of Compound I as described herein or a pharmaceutical composition comprising a salt of Compound I as described herein. In some embodiments, the cancer is non-small cell lung cancer that has been determined to comprise one or more atypical epidermal growth factor receptor (EGFR) mutations, and the individual is EGFR inhibitor naïve prior to administration to the individual of a salt of Compound I as described herein or a pharmaceutical composition comprising a salt of Compound I as described herein. In some embodiments, the cancer is metastatic non-small cell lung cancer that has been determined to comprise one or more atypical epidermal growth factor receptor (EGFR) mutations, and the individual is EGFR inhibitor naïve prior to administration to the individual of a salt of Compound I as described herein or a pharmaceutical composition comprising a salt of Compound I as described herein. In some embodiments, the cancer is non-small cell lung cancer comprising central nervous system (CNS) metastases (e.g., brain metastases) that has been determined to comprise one or more atypical epidermal growth factor receptor (EGFR) mutations, and the individual is EGFR inhibitor naïve prior to administration to the individual of a salt of Compound I as described herein or a pharmaceutical composition comprising a salt of Compound I as described herein In some embodiments, the salt of Compound I is the malonate salt. In some embodiments, the pharmaceutical composition comprises the malonate salt of Compound I.

In some embodiments of the methods disclosed herein the individual has received one or more prior therapy for treatment of the cancer prior to administration to the individual of Compound I, or a pharmaceutically acceptable salt thereof. In some embodiments, the one or more prior therapies exhibit inhibitory activity against cancer comprising a T790M mutation in the epidermal growth factor receptor (EGFR) protein. In some embodiments, the one or more prior therapies exhibiting inhibitory activity against cancer comprising a T790M mutation in the epidermal growth factor receptor (EGFR) protein comprises osimertinib. In some embodiments, the one or more prior therapies comprise one or more standard therapies for the cancer.

In some embodiments of the methods disclosed herein, the individual has received one or more prior EGFR inhibitor therapies for treatment of the cancer before administration to the individual of Compound I, or a pharmaceutically acceptable salt thereof. In some embodiments, the cancer is non-small cell lung cancer. In some embodiments, the cancer is non-small cell lung cancer that has been determined to comprise one or more atypical epidermal growth factor receptor (EGFR) mutations, and the individual has received one or more prior EGFR inhibitor therapies for treatment of the cancer prior to administration to the individual of a salt of Compound I as described herein or a pharmaceutical composition comprising a salt of Compound I as described herein. In some embodiments, the cancer is metastatic non-small cell lung cancer that has been determined to comprise one or more atypical epidermal growth factor receptor (EGFR) mutations, and the individual has received one or more prior EGFR inhibitor therapies for treatment of the cancer prior to administration to the individual of a salt of Compound I as described herein or a pharmaceutical composition comprising a salt of Compound I as described herein. In some embodiments, the cancer is non-small cell lung cancer comprising central nervous system (CNS) metastases (e.g., brain metastases) that has been determined to comprise one or more atypical epidermal growth factor receptor (EGFR) mutations, and the individual has received one or more prior EGFR inhibitor therapies for treatment of the cancer prior to administration to the individual of a salt of Compound I as described herein or a pharmaceutical composition comprising a salt of Compound I as described herein. In some embodiments, the salt of Compound I is the malonate salt. In some embodiments, the pharmaceutical composition comprises the malonate salt of Compound I.

In some embodiments, provided is a method of treating cancer in an individual in need thereof comprising administering to the individual a salt or pharmaceutical composition provided herein, wherein the cancer in the individual comprises one or more central nervous system (CNS) metastases (e.g., brain metastases). In some embodiments, the CNS metastases developed in the individual following prior therapy comprising an EGFR or HER2 inhibitor. In some embodiments, the prior therapy comprises an EGFR inhibitor. In some embodiments, the prior therapy comprises an EGFR exon 20 mutant inhibitor. In some embodiments, the prior therapy comprises an EGFR atypical mutation inhibitor. In some embodiments, the prior therapy comprises a HER2 inhibitor. In some embodiments, the prior therapy comprises a HER2 exon 20 mutant inhibitor. In some embodiments, the administration to the individual of a salt or pharmaceutical composition provided herein successfully treats the one or more one or more central nervous system (CNS) metastases (e.g., brain metastases).

In some embodiments of the methods disclosed herein, the individual has received one or more prior EGFR inhibitor therapies for treatment of the cancer before administration to the individual of Compound I, or a pharmaceutically acceptable salt thereof, and wherein the individual is further administered a bispecific EGF receptor-directed and MET receptor-directed antibody. In some embodiments, the cancer is non-small cell lung cancer. In some embodiments, the bispecific EGF receptor-directed and MET receptor-directed antibody is amivantamab. In some embodiments, the salt of Compound I is the malonate salt. In some embodiments, the pharmaceutical composition comprises the malonate salt of Compound I.

In some embodiments of the methods disclosed herein, (a) the cancer is non-small cell lung cancer and (b) the individual has received one or more prior EGFR inhibitor therapies for treatment of the cancer before administration to the individual of Compound I, or a pharmaceutically acceptable salt thereof, and wherein individual is further administered amivantamab. In some embodiments, the salt of Compound I is the malonate salt. In some embodiments, the pharmaceutical composition comprises the malonate salt of Compound I.

In some embodiments of the methods disclosed herein, the individual has received one or more prior therapies for treatment of the cancer before administration to the individual of Compound I, or a pharmaceutically acceptable salt thereof, and wherein the one or more prior therapies comprise an EGFR inhibitor and a bispecific EGF receptor-directed and MET receptor-directed antibody. In some embodiments, the cancer is non-small cell lung cancer. In some embodiments, the bispecific EGF receptor-directed and MET receptor-directed antibody is amivantamab. In some embodiments, the salt of Compound I is the malonate salt. In some embodiments, the pharmaceutical composition comprises the malonate salt of Compound I.

In some embodiments of the methods disclosed herein, the cancer is non-small cell lung cancer and the individual has received one or more prior therapies for treatment of the cancer before administration to the individual of Compound I, or a pharmaceutically acceptable salt thereof, and wherein the one or more prior therapies comprise an EGFR inhibitor and amivantamab. In some embodiments, the salt of Compound I is the malonate salt. In some embodiments, the pharmaceutical composition comprises the malonate salt of Compound I.

In some embodiments, the individual has been administered osimertinib prior to administration to the individual of a salt of Compound I as described herein or a pharmaceutical composition comprising a salt of Compound I as described herein. In some embodiments, the administration of osimertinib has been completed for at least about 1, 2, 3, 4, 5, 6, 7, 8, 9, 10, 11, or 12 months at the time when the individual is administered a salt of Compound I as described herein or a pharmaceutical composition comprising a salt of Compound I as described herein.

In some variations of any of the embodiments described herein, the individual has received one or more prior therapies for treatment of the cancer before administration to the individual of Compound I, or a pharmaceutically acceptable salt thereof, and wherein the one or more prior therapies comprises chemotherapy. In some embodiments, the one or more prior therapies comprising chemotherapy comprises platinum-based chemotherapy. In some embodiments, the one or more prior therapies comprising chemotherapy does not comprise platinum-based chemotherapy. In some embodiments, the salt of Compound I is the malonate salt. In some embodiments, the pharmaceutical composition comprises the malonate salt of Compound I.

In some embodiments of the methods disclosed herein, the individual has failed one or more prior therapies prior to administration to the individual of Compound I, or a pharmaceutically acceptable salt thereof, and wherein the one or more prior therapies is selected from (a) a bispecific EGF receptor-directed and MET receptor-directed antibody, and (b) chemotherapy. In some embodiments, the one or more prior therapies is a bispecific EGF receptor-directed and MET receptor-directed antibody. In some embodiments, the bispecific EGF receptor-directed and MET receptor-directed antibody is amivantamab. In some embodiments, the one or more prior therapies is chemotherapy. In some embodiments, the one or more prior therapies comprising chemotherapy comprises platinum-based chemotherapy. In some embodiments, the one or more prior therapies comprising chemotherapy does not comprise platinum-based chemotherapy. In some embodiments, the salt of Compound I is the malonate salt. In some embodiments, the pharmaceutical composition comprises the malonate salt of Compound I.

In some embodiments of the methods disclosed herein, the Compound I, or a pharmaceutically acceptable salt thereof, is administered to the individual in need thereof orally, parentally, intravenously, subcutaneously, or intracerebrally. In another embodiment is provided such methods, wherein a composition comprising Compound I, or a pharmaceutically acceptable salt thereof, is administered to the individual in need thereof orally, parentally, intravenously, subcutaneously, or intracerebrally.

In some embodiments, the method further comprises administering to the individual in need thereof one or more additional anticancer agents or a second therapy. In some embodiments, the one or more additional anticancer agents comprises one or more agents selected from the group consisting of HER2 inhibitors, HER2-CD3 bispecific antibodies, HER2-immune targeting bispecific antibodies, anti-HER2 chimeric antigen receptor (CAR) T cells, anti-HER2 chimeric antigen receptor (CAR) cytotoxic T-lymphocytes (CTLs), anti-HER2 chimeric antigen receptor (CAR) natural killer (NK) cells, anti-HER2 chimeric antigen receptor (CAR) cytokine-induced killer (CIK) cells, epidermal growth factor receptor (EGFR) inhibitors, poly-ADP-ribose polymerase (PARP) inhibitors, PD-1 inhibitors, PD-L1 inhibitors, CTLA-4 inhibitors, phosphoinositide 3-kinase (PI3K) inhibitors, and chemotherapeutic agents.

Exemplary HER2 inhibitors include, but are not limited to, trastuzumab, trastuzumab and hyaluronidase, trastuzumab and capecitabine, tucatinib, lapatinib, neratinib, dacomitinib, pertuzumab, margetuximab, trastuzumab emtansine, trastuzumab deruxtecan, ZW49 (Zymeworks), A166 (Klaus Pharma), ARX788 (Ambrx), RC48-ADC (RemeGen), vic-trastuzumab duocarmazine, zanidatamab (ZW25), zenocutuzamab (MCLA-128), ISB 1302, afatanib, poziotinib, pyrotinib, mobocertinib (TAK-788), and BDTX-189.

Exemplary EGFR inhibitors include, but are not limited to, erlotinib, osimertinib, neratinib, gefitinib, cetuximab, panitumumab, lapatinib, dacomitinib, necitumumab, vandetanib, afatinib, brigatinib, and icotinib.

Exemplary PARP inhibitors include, but are not limited to, niraparib, olaparib, talazoparib, rucaparib, veliparib, iniparib, pamiparib (BGB-290), CEP-9722, and E7016.

Exemplary PD-1 inhibitors include, but are not limited to, pembrolizumab, nivolumab, cemiplimab, JTX-4014, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, and AMP-514.

Exemplary PD-L1 inhibitors include, but are not limited to, atezolizumab, avelumab, durvalumab, KN035, CK-301, AUNP12, CA-170, and BMS-986189.

Exemplary CTLA-4 inhibitors include ipilimumab and tremelimumab.

In some embodiments, the one or more additional cancer agents are selected from the group consisting of bevacizumab, trastuzumab, trastuzumab and hyaluronidase, capecitabine, trastuzumab and capecitabine, tucatinib, lapatinib, neratinib, dacomitinib, pertuzumab, margetuximab, trastuzumab emtansine, trastuzumab deruxtecan, ZW49 (Zymeworks), A166 (Klaus Pharma), ARX788 (Ambrx), RC48-ADC (RemeGen), vic-trastuzumab duocarmazine, zanidatamab (ZW25), zenocutuzamab (MCLA-128), ISB 1302, afatanib, poziotinib, pyrotinib, mobocertinib (TAK-788), and BDTX-189, erlotinib, osimertinib, gefitinib, cetuximab, panitumumab, necitumumab, vandetanib, afatinib, brigatinib, icotinib, niraparib, olaparib, talazoparib, rucaparib, veliparib, iniparib, pamiparib (BGB-290), CEP-9722, E7016, pembrolizumab, nivolumab, cemiplimab, JTX-4014, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, atezolizumab, avelumab, durvalumab, KN035, CK-301, AUNP12, CA-170, and BMS-986189.

In some embodiments, the one or more additional cancer agents are selected from antibody-drug conjugates. In some embodiments, the antibody-drug conjugates are selected from the group consisting of trastuzumab emtansine and trastuzumab deruxtecan.

In some embodiments, the one or more additional anticancer agents comprises one or more chemotherapeutic agents. In some embodiments, the one or more chemotherapeutic agents is selected from the group consisting of doxorubicin, docetaxel, pemetrexed, paclitaxel, carboplatin, cisplatin, capecitabine, gemcitabine, vinorelbine, temozolomide, irinotecan, oxiplatin, and eribulin.

In some embodiments, the one or more additional anticancer agents comprise trastuzumab and capecitabine.

In some embodiments, the one or more additional anticancer agents comprise a PI3K inhibitor. Exemplary PI3K inhibitors include, but are not limited to, taselisib (GDC-0032), GDC-0077, perifosine, idelalisib, buparlisib (BKM120), duvelisib, (IPI-145), copanlisib (BAY 80-6946), PX-866, dactolisib, CUDC-907, voxtalisib (SAR245409, XL765), ME-401, IPI-549, SF1126, RP6530, INK1117, pictilisib (GDC-0941), XL147 (SAR245408), palomid 529, GSK1059615, ZSTK474, and PWT33597.

In some embodiments, the one or more additional cancer agents are selected from the group consisting of erlotinib, gefitinib, afatanib, bevacizumab, trastuzumab, trastuzumab and hyaluronidase, capecitabine, trastuzumab and capecitabine, tucatinib, lapatinib, neratinib, dacomitinib, pertuzumab, margetuximab, trastuzumab emtansine, trastuzumab deruxtecan, ZW49 (Zymeworks), A166 (Klaus Pharma), ARX788 (Ambrx), RC48-ADC (RemeGen), vic-trastuzumab duocarmazine, zanidatamab (ZW25), zenocutuzamab (MCLA-128), ISB 1302, poziotinib, pyrotinib, mobocertinib (TAK-788), and BDTX-189, osimertinib, cetuximab, panitumumab, necitumumab, vandetanib, brigatinib, icotinib, niraparib, olaparib, talazoparib, rucaparib, veliparib, iniparib, pamiparib (BGB-290), CEP-9722, E7016, pembrolizumab, nivolumab, cemiplimab, JTX-4014, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, atezolizumab, avelumab, durvalumab, KN035, CK-301, AUNP12, CA-170, and BMS-986189.

In some embodiments, the one or additional cancer agents are selected from the group consisting of osimertinib, erlotinib, gefitinib, and afatinib.

In some embodiments, the one or additional cancer agents comprise osimertinib.

In some embodiments, the one or additional cancer agents comprise erlotinib.

In some embodiments, the one or additional cancer agents comprise gefitinib.

In some embodiments, the one or additional cancer agents comprise afatinib.

In some variations of any of the embodiments described herein, the method further comprises administering to the individual in need thereof one or more additional anticancer agents, wherein the one or more additional anticancer agents comprises a bispecific EGF receptor-directed and MET receptor-directed antibody. In some embodiments, the bispecific EGF receptor-directed and MET receptor-directed antibody is amivantamab.

In some embodiments, the method further comprises treating the individual in need thereof with radiation.

In some variations, the cancer comprises a mutation in phosphatidylinositol-4,5-bisphosphate 3-kinase catalytic subunit alpha (PIK3CA) protein. In some variations, the cancer does not comprise a mutation in phosphatidylinositol-4,5-bisphosphate 3-kinase catalytic subunit alpha (PIK3CA) protein. In some variations, the cancer does not comprise a mutation in phosphatidylinositol-4,5-bisphosphate 3-kinase catalytic subunit alpha (PIK3CA) protein at histidine 1047. In some variations, the cancer does not comprise a mutation in phosphatidylinositol-4,5-bisphosphate 3-kinase catalytic subunit alpha (PIK3CA) protein selected from H1047L and H1047R.

In some variations of any of the embodiments described herein, the cancer comprises one or more (such as 1, 2, or 3) mutations in the epidermal growth factor receptor (EGFR) protein. In some variations, the one or more mutations in the EGFR protein comprises one or more (such as 1, 2, or 3) mutations selected from the group consisting of G309A, G309E, S310F, R678Q, R678Q and L755W, L755S, L755W, 1767M, D769H, D769Y, V777L, Y835F, V842I, R896C, G1201V, del.755-759EGFR Del19/T790M, EGFR L858R/T790M, EGFR L858R, EGFR del 19, EGFR L858R/C797S, EGFR del19/C797S, EGFR Exon20 ins NPH, EGFR Exon20 ins SVD, EGFR Exon20 ins FQEA, EGFR Exon20 ins H, and EGFR Exon20 ins ASV. In some variations, the cancer comprises one or more mutations selected from the group consisting of G309A, G309E, S310F, R678Q, R678Q and L755W, L755S, L755W, 1767M, D769H, D769Y, V777L, Y835F, V842I, R896C, G1201V, del.755-759EGFR Del19/T790M, EGFR L858R/T790M, EGFR L858R, EGFR del 19, EGFR L858R/C797S, EGFR del19/C797S, EGFR Exon20 ins NPH, EGFR Exon20 ins SVD, EGFR Exon20 ins FQEA, EGFR Exon20 ins H, and EGFR Exon20 ins ASV. In some variations, the cancer does not comprise any of the EGFR mutations described above. In some variations, the cancer does not comprise any mutation in the epidermal growth factor receptor (EGFR) protein.

In some variations of any of the embodiments described herein, the cancer comprises one or more (such as 1, 2, or 3) mutations in the HER2 protein, wherein the one or more (such as 1, 2, or 3) mutations in the HER2 protein is selected from p.A775_G776insYVMA, p.778insGCP, p.G780_P781dupGSP, and p.G778_S779insCPG. In some variations, the cancer does not comprise any mutation in the HER2 protein. In some variations, the cancer does not comprise one or more mutations in the HER2 protein. In some variations, the cancer does not comprise one or more exon 20 insertion mutations in the HER2 protein. In some variations, the cancer does not comprise one or more exon 20 insertion mutations in the HER2 protein, selected from p.A775_G776insYVMA, p.778insGCP, p.G780_P781dupGSP, and p.G778_S779insCPG. In some variations, the cancer does not comprise an exon 20 insertion mutation in the HER2 protein that is p.A775_G776insYVMA. In some variations, the cancer does not comprise an exon 20 insertion mutation in the HER2 protein that is p.778insGCP. In some variations, the cancer does not comprise an exon 20 insertion mutation in the HER2 protein that is p.G780_P781dupGSP. In some variations, the cancer does not comprise an exon 20 insertion mutation in the HER2 protein that is p.G778_S779insCPG. In some variations, the cancer does not comprise a mutation in the HER2 protein that is L775S. In some variations, the cancer does not comprise a mutation in the HER2 protein that is G776C.

In some embodiments, the individual is a mammal. In some embodiments, the individual is a human.

### Determination of EGFR Mutational Status

In some aspects of the methods disclosed herein, the EGFR mutational status of a cancer in an individual involve is derived from analysis of a biological sample obtained from the individual. The methods of obtaining such a biological sample may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from lung tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. In some aspects, a sample is a cancerous or non-cancerous lung tissue sample. Alternatively, the sample may be obtained from any other source including but not limited to blood, sweat, hair follicle, buccal tissue, tears, menses, feces, or saliva. A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein.

In some cases, a biological sample is a cell-free sample (e.g., a serum sample). In such cases, a biological sample may contain cell-free nucleic acids such as DNA (e.g., cell-free tumor DNA, cell-free fetal DNA) or RNA (e.g., cell-free tumor RNA, cell-free fetal RNA). In some aspects, a cell-free biological sample contains, or is suspected of containing, DNA or RNA from lung cancer.

The EGFR mutational status of a cancer in an individual may be determined by analyzing the sample of biological materials derived from an individual by using commercial providers and/or methods known to those having ordinary skill in the art. Examples of such methods include, but are not limited to, next-generation sequencing (NGS) technologies [including Oncomine Dx target test (ODxTT; Thermo Fisher Scientific); Guardant360 TissueNext (Guardant Health); and GuardantOMNI (Guardant Health)], RT-PCR [including the RT-qPCR-based cobas EGFR mutation test v2 (cobas EGFR: Roche Molecular Systems)], fluorescence in situ hybridization (FISH), immunohistochemistry (IHC), polony sequencing, Illumina (Solexa) sequencing, sequencing by oligonucleotide ligation and detection (SOLiD) sequencing, ion torrent semiconductor sequencing, DNA nanoball sequencing, Heliscope single molecule sequencing, and single molecule real time (SMRT) sequencing. Some of these methods are described in Dalurzo et al., OncoTargets and Therapy, 2021, vol. 14, pp. 4671-4692, which is hereby incorporated by reference in its entirety.

In some embodiments, methods involve amplifying and/or sequencing one or more target genomic regions using at least one pair of primers specific to the target genomic regions. In other embodiments, enzymes are added such as primases or primase/polymerase combination enzyme to the amplification step to synthesize primers.

In some embodiments, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991, each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

Other assays that may be used to analyze nucleic acids in order to determine the EGFR mutational status of a biological sample derived from an individual in need thereof that are known to those of ordinary skill in the art include, but are not limited to, nucleic amplification, polymerase chain reaction, quantitative PCR, RT-PCR, in situ hybridization, digital PCR, ddPCR (droplet digital PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### Dosing and Method of Administration

The amount of Compound I, or a pharmaceutically acceptable salt thereof, as described herein administered to an individual (e.g., a human) may vary with the particular composition, the method of administration, and the particular cancer being treated. The amount should be sufficient to produce a desirable response, such as a therapeutic response against the cancer. In some embodiments, the amount of Compound I, or a pharmaceutically acceptable salt thereof, as described herein is below the level that induces a toxicological effect (e.g., an effect above a clinically acceptable level of toxicity) or is at a level where a potential side effect can be controlled or tolerated when a salt of Compound I as described herein is administered to the individual.

In some embodiments, Compound I, or a pharmaceutically acceptable salt thereof, as described herein is administered to a subject systemically. In some embodiments, Compound I, or a pharmaceutically acceptable salt thereof, as described herein is administered to a subject parenterally. In some embodiments, Compound I, or a pharmaceutically acceptable salt thereof, as described herein is administered to a subject topically (i.e., locally). In some embodiments, Compound I, or a pharmaceutically acceptable salt thereof, as described herein is administered to a subject orally, intravenously, intraarterially, intraperitoneally, intravesicularly, subcutaneously, intrathecally, intrapulmonarily, intramuscularly, intratracheally, intracerebrally, intraocularly, transdermally, or by inhalation. In some embodiments, Compound I, or a pharmaceutically acceptable salt thereof, as described herein is administered to a subject orally. In some embodiments, Compound I, or a pharmaceutically acceptable salt thereof, as described herein is administered to a subject orally, while one or more additional agents may be administered to the subject by other routes, including intravenously, intraarterially, intraperitoneally, intravesicularly, subcutaneously, intrathecally, intrapulmonarily, intramuscularly, intratracheally, intracerebrally, intraocularly, transdermally, or by inhalation. For example, in some embodiments, Compound I, or a pharmaceutically acceptable salt thereof, as described herein is administered to a subject orally, while one or more additional agents are administered to the subject intravenously. In some embodiments, Compound I, or a pharmaceutically acceptable salt thereof, as described herein is orally administered to the individual. In some embodiments, Compound I, or a pharmaceutically acceptable salt thereof, as described herein is parenterally administered to the individual. In some embodiments, Compound I, or a pharmaceutically acceptable salt thereof, as described herein is intravenously administered to the individual. In some embodiments, Compound I, or a pharmaceutically acceptable salt thereof, as described herein is subcutaneously administered to the individual. In some embodiments Compound I, or a pharmaceutically acceptable salt thereof, as described herein is intracerebrally administered to the individual.

In some embodiments, Compound I, or a pharmaceutically acceptable salt thereof, as described herein and a second agent or therapy are administered simultaneously to the individual. In some embodiments, Compound I, or a pharmaceutically acceptable salt thereof, as described herein and a second agent or therapy are administered concurrently to the individual. In some embodiments, Compound I, or a pharmaceutically acceptable salt thereof, as described herein and a second agent or therapy are administered sequentially to the individual.

In some embodiments, a dosage of from about 1 mg/kg to about 100 mg/kg of Compound I, or a pharmaceutically acceptable salt thereof, as described herein is administered into an individual, such as a human (e.g., from about 1 mg/kg to about 75 mg/kg, from about 2 mg/kg to about 75 mg/kg, from about 3 mg/kg to about 75 mg/kg, from about 4 mg/kg to about 75 mg/kg, from about 5 mg/kg to about 75 mg/kg, from about 1 mg/kg to about 50 mg/kg, from about 2 mg/kg to about 50 mg/kg, from about 3 mg/kg to about 50 mg/kg, from about 4 mg/kg to about 50 mg/kg, from about 5 mg/kg to about 50 mg/kg, from about 1 mg/kg to about 40 mg/kg, from about 2 mg/kg to about 40 mg/kg, from about 3 mg/kg to about 40 mg/kg, from about 4 mg/kg to about 40 mg/kg, from about 5 mg/kg to about 40 mg/kg, from about 1 mg/kg to about 30 mg/kg, from about 2 mg/kg to about 30 mg/kg, from about 3 mg/kg to about 30 mg/kg, from about 4 mg/kg to about 30 mg/kg, from about 5 mg/kg to about 30 mg/kg, from about 6 mg/kg to about 80 mg/kg, from about 10 mg/kg to about 80 mg/kg, from about 15 mg/kg to about 80 mg/kg, from about 20 mg/kg to about 80 mg/kg, from about 25 mg/kg to about 80 mg/kg, from about 30 mg/kg to about 80 mg/kg, from about 35 mg/kg to about 80 mg/kg, from about 40 mg/kg to about 80 mg/kg, from about 45 mg/kg to about 80 mg/kg, or from about 50 mg/kg to about 80 mg/kg). In some embodiments, a salt of Compound I as described herein is administered to an individual (e.g., human) once or twice daily, optionally orally or parentally. For oral administration, an exemplary daily dose of Compound I, or a pharmaceutically acceptable salt thereof, as described herein will be from about 0.001 to about 1000 mg/kg of body weight, with courses of treatment repeated at appropriate intervals.

Furthermore, Compound I, or a pharmaceutically acceptable salt thereof, as described herein may be administered according to the methods disclosed herein in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg, or from about 10 mg to about 750 mg, or from about 10 mg to about 700 mg, or from about 10 mg to about 650 mg, or from about 10 mg to about 600 mg, or from about 10 mg to about 575 mg, or from about 10 mg to about 550 mg, or from about 10 mg to about 500 mg, or from about 10 mg to about 450 mg, or from about 10 mg to about 400 mg, or from about 10 mg to about 350 mg, or from about 10 mg to about 300 mg, or from about 10 mg to about 275 mg, or from about 10 mg to about 250 mg, or from about 10 mg to about 200 mg, or from about 10 mg to about 150 mg, or from about 10 mg to about 100 mg, or from about 10 mg to about 75 mg, or from about 10 mg to about 50 mg, or from about 10 mg to about 25 mg.

Furthermore, Compound I, or a pharmaceutically acceptable salt thereof, as described herein may be administered according to the methods disclosed herein once per day (QD). Compound I, or a pharmaceutically acceptable salt thereof, may be administered in an amount that provides an amount of the free base of Compound I, of about 6 mg QD, about 12 mg QD, about 20 mg QD, about 30 mg QD, about 45 mg QD, about 60 mg QD, about 75 mg QD, about 90 mg QD, or about 105 mg QD. In one embodiment, Compound I, or a pharmaceutically acceptable salt thereof, may be administered in an amount that provides an amount of the free base of Compound I of about 6 mg QD. In another embodiment, Compound I, or a pharmaceutically acceptable salt thereof, may be administered in an amount that provides an amount of the free base of Compound I of about 12 mg QD. In another embodiment, Compound I, or a pharmaceutically acceptable salt thereof, may be administered in an amount that provides an amount of the free base of Compound I of about 20 mg QD. In another embodiment, Compound I, or a pharmaceutically acceptable salt thereof, may be administered in an amount that provides an amount of the free base of Compound I of about 30 mg QD. In another embodiment, Compound I, or a pharmaceutically acceptable salt thereof, may be administered in an amount that provides an amount of the free base of Compound I of about 45 mg QD. In another embodiment, Compound I, or a pharmaceutically acceptable salt thereof, may be administered in an amount that provides an amount of the free base of Compound I of about 60 mg QD. In another embodiment, Compound I, or a pharmaceutically acceptable salt thereof, may be administered in an amount that provides an amount of the free base of Compound I of about 75 mg QD. In another embodiment, Compound I, or a pharmaceutically acceptable salt thereof, may be administered in an amount that provides an amount of the free base of Compound I of about 90 mg QD. In another embodiment, Compound I, or a pharmaceutically acceptable salt thereof, may be administered in an amount that provides an amount of the free base of Compound I of about 105 mg QD.

In another embodiment, Compound I may be administered as a salt in an amount that provides an amount of the free base of Compound I of about 110 mg QD, or about 115 mg QD, or about 120 mg QD, or about 125 mg QD, or about 130 mg QD, or about 135 mg QD, or about 140 mg QD, or about 145 mg QD, or about 150 mg QD, or about 155 mg QD, or about 160 mg QD, or about 165 mg QD, or about 170 mg QD, or about 180 mg QD, or about 190 mg QD, or about 200 mg QD. In some embodiments, Compound I as administered in the form of a malonate salt in an amount that provides an amount of the free base of Compound I of about 110 mg QD, or about 115 mg QD, or about 120 mg QD, or about 125 mg QD, or about 130 mg QD, or about 135 mg QD, or about 140 mg QD, or about 145 mg QD, or about 150 mg QD, or about 155 mg QD, or about 160 mg QD, or about 165 mg QD, or about 170 mg QD, or about 180 mg QD, or about 190 mg QD, or about 200 mg QD. In some embodiments, the salt of Compound I is administered in the form of a pharmaceutically acceptable composition. In some embodiments, the salt of Compound I is the malonate salt. In some embodiments, the salt of Compound I is the glycolate salt.

Furthermore, Compound I, or a pharmaceutically acceptable salt thereof, as described herein may be administered according to the methods disclosed herein twice per day (BID). In another embodiment, Compound I, or a pharmaceutically acceptable salt thereof, may be administered in an amount that provides an amount of the free base of Compound I, of about 3 mg BID, about 6 mg BID, about 10 mg BID, about 15 mg BID, about 20 mg BID, about 25 mg BID, about 30 mg BID, about 45 mg BID, or about 50 mg BID. In another embodiment, Compound I, or a pharmaceutically acceptable salt thereof, may be administered in an amount that provides an amount of the free base of Compound I of about 3 mg BID. In another embodiment, Compound I, or a pharmaceutically acceptable salt thereof, may be administered in an amount that provides an amount of the free base of Compound I of about 6 mg BID. In another embodiment, Compound I, or a pharmaceutically acceptable salt thereof, may be administered in an amount that provides an amount of the free base of Compound I of about 10 mg BID. In another embodiment, Compound I, or a pharmaceutically acceptable salt thereof, may be administered in an amount that provides an amount of the free base of Compound I of about 15 mg BID. In another embodiment, Compound I, or a pharmaceutically acceptable salt thereof, may be administered in an amount that provides an amount of the free base of Compound I of about 20 mg BID. In another embodiment, Compound I, or a pharmaceutically acceptable salt thereof, may be administered in an amount that provides an amount of the free base of Compound I of about 25 mg BID. In another embodiment, Compound I, or a pharmaceutically acceptable salt thereof, may be administered in an amount that provides an amount of the free base of Compound I of about 30 mg BID. In another embodiment, Compound I, or a pharmaceutically acceptable salt thereof, may be administered in an amount that provides an amount of the free base of Compound I of about 45 mg BID. In another embodiment, Compound I, or a pharmaceutically acceptable salt thereof, may be administered in an amount that provides an amount of the free base of Compound I of about 50 mg BID.

Furthermore, Compound I, or a pharmaceutically acceptable salt thereof, as described herein may be administered according to the methods disclosed herein in an amount that provides a total daily dose of Compound I selected from (a) more than 10 mg and less than about 45 mg, (b) more than about 45 mg but less than about 200 mg, (c) from about 45 mg to about 60 mg, (d) from about 45 mg to about 90 mg, (e) from about 45 mg to about 120 mg, (f) about 45 mg, (g) about 60 mg, (h) about 75 mg, (i) about 80 mg, (j) about 90 mg, (k) about 100 mg, (l) about 120 mg, (m) about 150 mg, (n) about 175 mg, (o) about 200 mg, and (p) greater than about 120 mg but less than about 200 mg. In some embodiments, the salt of Compound I is the malonate salt. In some embodiments, the pharmaceutical composition comprises the malonate salt of Compound I.

Furthermore, Compound I may be administered in the form of a malonate salt according to the methods disclosed herein in an amount that provides a total daily dose of Compound I selected from (a) more than 10 mg and less than about 45 mg, (b) more than about 45 mg but less than about 200 mg, (c) from about 45 mg to about 60 mg, (d) from about 45 mg to about 90 mg, (e) from about 45 mg to about 120 mg, (f) about 45 mg, (g) about 60 mg, (h) about 75 mg, (i) about 80 mg, (j) about 90 mg, (k) about 100 mg, (l) about 120 mg, (m) about 150 mg, (n) about 175 mg, (o) about 200 mg, and (p) greater than about 120 mg but less than about 200 mg. In some embodiments, the individual in need thereof is administered the malonate salt in an amount that provides a total daily dose of Compound I of greater than about 45 mg but less than about 200 mg. In some embodiments, the individual in need thereof is administered the malonate salt in an amount that provides a total daily dose of Compound I of greater than about 45 mg but less than about 175 mg. In some embodiments, the individual in need thereof is administered the malonate salt in an amount that provides a total daily dose of Compound I of greater than about 45 mg but less than about 150 mg. In some embodiments, the individual in need thereof is administered the malonate salt in an amount that provides a total daily dose of Compound I of greater than about 45 mg but less than about 140 mg. In some embodiments, the individual in need thereof is administered the malonate salt in an amount that provides a total daily dose of Compound I of greater than about 45 mg but less than about 130 mg. In some embodiments, the individual in need thereof is administered the malonate salt in an amount that provides a total daily dose of Compound I of greater than about 45 mg but less than about 120 mg. In some embodiments, the individual in need thereof is administered the malonate salt in an amount that provides a total daily dose of Compound I of greater than about 45 mg but less than about 110 mg. In some embodiments, the individual in need thereof is administered the malonate salt in an amount that provides a total daily dose of Compound I of greater than about 45 mg but less than about 100 mg. In some embodiments, the individual in need thereof is administered the malonate salt in an amount that provides a total daily dose of Compound I of greater than about 45 mg but less than about 95 mg. In some embodiments, the individual in need thereof is administered the malonate salt in an amount that provides a total daily dose of Compound I of greater than about 45 mg but less than about 90 mg. In some embodiments, the individual in need thereof is administered the malonate salt in an amount that provides a total daily dose of Compound I of greater than about 45 mg but less than about 85 mg. In some embodiments, the individual in need thereof is administered the malonate salt in an amount that provides a total daily dose of Compound I of greater than about 45 mg but less than about 80 mg. In some embodiments, the individual in need thereof is administered the malonate salt in an amount that provides a total daily dose of Compound I of greater than about 45 mg but less than about 75 mg.

Furthermore, Compound I may be administered in the form of a malonate salt according to the methods disclosed herein in an amount that is about 30 mg BID, about 40 mg BID, about 45 mg QD, about 50 mg BID, about 60 mg QD, about 75 mg QD, about 90 mg QD, or about 120 mg QD. In some embodiments, the individual in need thereof is administered the malonate salt or the pharmaceutical composition in an amount that is about 30 mg BID. In another embodiment, the individual in need thereof is administered the malonate salt or the pharmaceutical composition in an amount that is about 40 mg BID. In another embodiment, the individual in need thereof is administered the malonate salt or the pharmaceutical composition in an amount that is about 45 mg QD. In another embodiment, the individual in need thereof is administered the malonate salt or the pharmaceutical composition in an amount that is about 50 mg BID. In another embodiment, the individual in need thereof is administered the malonate salt or the pharmaceutical composition in an amount that is about 60 mg QD. In another embodiment, the individual in need thereof is administered the malonate salt or the pharmaceutical composition in an amount that is about 75 mg QD. In another embodiment, the individual in need thereof is administered the malonate salt or the pharmaceutical composition in an amount that is about 80 mg. In another embodiment, the individual in need thereof is administered the malonate salt or the pharmaceutical composition in an amount that is about 85 mg. In another embodiment, the individual in need thereof is administered the malonate salt or the pharmaceutical composition in an amount that is about 90 mg QD. In another embodiment, the individual in need thereof is administered the malonate salt or the pharmaceutical composition in an amount that is 100 mg. In another embodiment, the individual in need thereof is administered the malonate salt or the pharmaceutical composition in an amount that is 110 mg. In another embodiment, the individual in need thereof is administered the malonate salt or the pharmaceutical composition in an amount that about 120 mg QD. In another embodiment, the individual in need thereof is administered the malonate salt or the pharmaceutical composition in an amount that is about 125 mg. In another embodiment, the individual in need thereof is administered the malonate salt or the pharmaceutical composition in an amount that is about 130 mg. In another embodiment, the individual in need thereof is administered the malonate salt or the pharmaceutical composition in an amount that is about 135 mg. In another embodiment, the individual in need thereof is administered the malonate salt or the pharmaceutical composition in an amount that is about 140 mg. In another embodiment, the individual in need thereof is administered the malonate salt or the pharmaceutical composition in an amount that is about 145 mg. In another embodiment, the individual in need thereof is administered the malonate salt or the pharmaceutical composition in an amount that is about 150 mg. In another embodiment, the individual in need thereof is administered the malonate salt or the pharmaceutical composition in an amount that is about 160 mg. In another embodiment, the individual in need thereof is administered the malonate salt or the pharmaceutical composition in an amount that is 170 mg. In another embodiment, the individual in need thereof is administered the malonate salt or the pharmaceutical composition in an amount that is about 180 mg. In another embodiment, the individual in need thereof is administered the malonate salt or the pharmaceutical composition in an amount that is 190 mg. In another embodiment, the individual in need thereof is administered the malonate salt or the pharmaceutical composition in an amount that is about 200 mg.

In another embodiment are provided the methods disclosed herein, wherein the individual is administered Compound I, or a pharmaceutically acceptable salt thereof, in an amount that provides a mean unbound concentration of Compound I in the plasma of the individual that is greater than or equal to 0.1 nM for at least 8 hours following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 8 hours following the administration to the individual of Compound I, or a pharmaceutically acceptable salt thereof. In some embodiments, the salt of Compound I is the malonate salt.

In another embodiment, the individual in need thereof is administered Compound I, or a pharmaceutically acceptable salt thereof, in an amount that provides a mean unbound concentration of Compound I in the plasma of the individual that is greater than or equal to 0.1 nM for at least 12 hours following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 12 hours following the administration to the individual of Compound I, or a pharmaceutically acceptable salt thereof. In some embodiments, the salt of Compound I is the malonate salt.

In another embodiment, the individual in need thereof is administered Compound I, or a pharmaceutically acceptable salt thereof, in an amount that provides a mean unbound concentration of Compound I in the plasma of the individual that is greater than or equal to 0.1 nM for at least 16 hours following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 16 hours following the administration to the individual of Compound I, or a pharmaceutically acceptable salt thereof. In some embodiments, the salt of Compound I is the malonate salt.

In another embodiment, the individual in need thereof is administered Compound I, or a pharmaceutically acceptable salt thereof, in an amount that provides a mean unbound concentration of Compound I in the plasma of the individual that is greater than or equal to 0.1 nM for at least 20 hours following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 20 hours following the administration to the individual of Compound I, or a pharmaceutically acceptable salt thereof. In some embodiments, the salt of Compound I is the malonate salt.

In another embodiment, the individual in need thereof is administered Compound I, or a pharmaceutically acceptable salt thereof, in an amount that provides a mean unbound concentration of Compound I in the plasma of the individual that is greater than or equal to 0.1 nM for at least 24 hours following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 24 hours following the administration to the individual of Compound I, or a pharmaceutically acceptable salt thereof. In some embodiments, the salt of Compound I is the malonate salt.

In another embodiment, the individual in need thereof is administered Compound I, or a pharmaceutically acceptable salt thereof, in an amount that provides a Cmax of Compound I in the plasma of the individual that is greater than or equal to 0.2 nM following the administration to the individual of Compound I, or a pharmaceutically acceptable salt thereof. In another embodiment, the individual in need thereof is administered Compound I, or a pharmaceutically acceptable salt thereof, in an amount that provides a Cmax of Compound I in the plasma of the individual that is greater than or equal to 0.25 nM, or greater than or equal to 0.3 nM, or greater than or equal to 0.35 nM, or greater than or equal to 0.4 nM, or greater than or equal to 0.45 nM, or greater than or equal to 0.5 nM, or greater than or equal to 0.55 nM, or greater than or equal to 0.6 nM, or greater than or equal to 0.65 nM, or greater than or equal to 0.7 nM, or greater than or equal to 0.75 nM, or greater than or equal to 0.8 nM, or greater than or equal to 0.85 nM, or greater than or equal to 0.9 nM, or greater than or equal to 0.95 nM, or greater than or equal to 1 nM, or greater than or equal to 1.25 nM, or greater than or equal to 1.5 nM, or greater than or equal to 1.75 nM, or greater than or equal to 2 nM, or greater than or equal to 2.5 nM, or greater than or equal to 3 nM, or greater than or equal to 3.5 nM, or greater than or equal to 4 nM, or greater than or equal to 4.5 nM, or greater than or equal to 5 nM, or greater than or equal to 5.5 nM, or greater than or equal to 6 nM, or greater than or equal to 6.5 nM, or greater than or equal to 7 nM, or greater than or equal to 7.5 nM, or greater than or equal to 8 nM, or greater than or equal to 8.5 nM, or greater than or equal to 9 nM, or greater than or equal to 9.5 nM, or greater than or equal to 10 nM following the administration to the individual of Compound I, or a pharmaceutically acceptable salt thereof. In some embodiments, the salt of Compound I is the malonate salt.

In another embodiment, the individual in need thereof is administered Compound I, or a pharmaceutically acceptable salt thereof, in an amount that provides a Cmax of Compound I in the plasma of the individual that is greater than or equal to 0.2 nM and less than or equal to 10 nM following the administration to the individual of Compound I, or a pharmaceutically acceptable salt thereof. In another embodiment, the individual in need thereof is administered Compound I, or a pharmaceutically acceptable salt thereof, in an amount that provides a Cmax of Compound I in the plasma of the individual that is greater than or equal to 0.2 nM and less than or equal to 9 nM, or greater than or equal to 0.2 nM and less than or equal to 8.5 nM, or greater than or equal to 0.2 nM and less than or equal to 8 nM, or greater than or equal to 0.2 nM and less than or equal to 7.5 nM, or greater than or equal to 0.2 nM and less than or equal to 7 nM, or greater than or equal to 0.2 nM and less than or equal to 6.5 nM, or greater than or equal to 0.2 nM and less than or equal to 6 nM, or greater than or equal to 0.2 nM and less than or equal to 5.5 nM, or greater than or equal to 0.2 nM and less than or equal to 5 nM, or greater than or equal to 0.2 nM and less than or equal to 4.5 nM, or greater than or equal to 0.2 nM and less than or equal to 4 nM, or greater than or equal to 0.2 nM and less than or equal to 3.5 nM, greater than or equal to 0.2 nM and less than or equal to 3 nM, or greater than or equal to 0.2 nM and less than or equal to 2.75 nM following the administration to the individual of Compound I, or a pharmaceutically acceptable salt thereof. In some embodiments, the salt of Compound I is the malonate salt.

In another embodiment, the individual in need thereof is administered Compound I, or a pharmaceutically acceptable salt thereof, in an amount that provides in the plasma of the individual following the administration to the individual of Compound I, or a pharmaceutically acceptable salt thereof, (a) a Cmax of Compound I that is greater than or equal to 0.2 nM and less than or equal to 10 nM, and (b) a mean unbound concentration of Compound I that is greater than or equal to 0.2 nM and less than or equal to 10 nM for at least 8 hours. In another embodiment, the individual in need thereof is administered Compound I, or a pharmaceutically acceptable salt thereof, in an amount that provides a Cmax of Compound I in the plasma of the individual that is greater than or equal to 0.2 nM and less than or equal to 9 nM, or greater than or equal to 0.2 nM and less than or equal to 8.5 nM, or greater than or equal to 0.2 nM and less than or equal to 8 nM, or greater than or equal to 0.2 nM and less than or equal to 7.5 nM, or greater than or equal to 0.2 nM and less than or equal to 7 nM, or greater than or equal to 0.2 nM and less than or equal to 6.5 nM, or greater than or equal to 0.2 nM and less than or equal to 6 nM, or greater than or equal to 0.2 nM and less than or equal to 5.5 nM, or greater than or equal to 0.2 nM and less than or equal to 5 nM, or greater than or equal to 0.2 nM and less than or equal to 4.5 nM, or greater than or equal to 0.2 nM and less than or equal to 4 nM, or greater than or equal to 0.2 nM and less than or equal to 3.5 nM, greater than or equal to 0.2 nM and less than or equal to 3 nM, or greater than or equal to 0.2 nM and less than or equal to 2.75 nM following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 8 hours following the administration to the individual of Compound I, or a pharmaceutically acceptable salt thereof. In some embodiments, the salt of Compound I is the malonate salt. It is understood that the disclosure herein contemplates any combinations of the values disclosed for Cmax with any values disclosed herein for mean unbound concentration of Compound I.

In another embodiment, the individual in need thereof is administered Compound I, or a pharmaceutically acceptable salt thereof, in an amount that provides in the plasma of the individual following the administration to the individual of Compound I, or a pharmaceutically acceptable salt thereof, (a) a Cmax of Compound I that is greater than or equal to 0.2 nM and less than or equal to 10 nM, and (b) a mean unbound concentration of Compound I that is greater than or equal to 0.2 nM and less than or equal to 10 nM for at least 12 hours. In another embodiment, the individual in need thereof is administered Compound I, or a pharmaceutically acceptable salt thereof, in an amount that provides a Cmax of Compound I in the plasma of the individual that is greater than or equal to 0.2 nM and less than or equal to 9 nM, or greater than or equal to 0.2 nM and less than or equal to 8.5 nM, or greater than or equal to 0.2 nM and less than or equal to 8 nM, or greater than or equal to 0.2 nM and less than or equal to 7.5 nM, or greater than or equal to 0.2 nM and less than or equal to 7 nM, or greater than or equal to 0.2 nM and less than or equal to 6.5 nM, or greater than or equal to 0.2 nM and less than or equal to 6 nM, or greater than or equal to 0.2 nM and less than or equal to 5.5 nM, or greater than or equal to 0.2 nM and less than or equal to 5 nM, or greater than or equal to 0.2 nM and less than or equal to 4.5 nM, or greater than or equal to 0.2 nM and less than or equal to 4 nM, or greater than or equal to 0.2 nM and less than or equal to 3.5 nM, greater than or equal to 0.2 nM and less than or equal to 3 nM, or greater than or equal to 0.2 nM and less than or equal to 2.75 nM following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 12 hours following the administration to the individual of Compound I, or a pharmaceutically acceptable salt thereof. In some embodiments, the salt of Compound I is the malonate salt. It is understood that the disclosure herein contemplates any combinations of the values disclosed for Cmax with any values disclosed herein for mean unbound concentration of Compound I.

In another embodiment, the individual in need thereof is administered Compound I, or a pharmaceutically acceptable salt thereof, in an amount that provides in the plasma of the individual following the administration to the individual of Compound I, or a pharmaceutically acceptable salt thereof, (a) a Cmax of Compound I that is greater than or equal to 0.2 nM and less than or equal to 10 nM, and (b) a mean unbound concentration of Compound I that is greater than or equal to 0.2 nM and less than or equal to 10 nM for at least 16 hours. In another embodiment, the individual in need thereof is administered Compound I, or a pharmaceutically acceptable salt thereof, in an amount that provides a Cmax of Compound I in the plasma of the individual that is greater than or equal to 0.2 nM and less than or equal to 9 nM, or greater than or equal to 0.2 nM and less than or equal to 8.5 nM, or greater than or equal to 0.2 nM and less than or equal to 8 nM, or greater than or equal to 0.2 nM and less than or equal to 7.5 nM, or greater than or equal to 0.2 nM and less than or equal to 7 nM, or greater than or equal to 0.2 nM and less than or equal to 6.5 nM, or greater than or equal to 0.2 nM and less than or equal to 6 nM, or greater than or equal to 0.2 nM and less than or equal to 5.5 nM, or greater than or equal to 0.2 nM and less than or equal to 5 nM, or greater than or equal to 0.2 nM and less than or equal to 4.5 nM, or greater than or equal to 0.2 nM and less than or equal to 4 nM, or greater than or equal to 0.2 nM and less than or equal to 3.5 nM, greater than or equal to 0.2 nM and less than or equal to 3 nM, or greater than or equal to 0.2 nM and less than or equal to 2.75 nM following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 16 hours following the administration to the individual of Compound I, or a pharmaceutically acceptable salt thereof. In some embodiments, the salt of Compound I is the malonate salt. It is understood that the disclosure herein contemplates any combinations of the values disclosed for Cmax with any values disclosed herein for mean unbound concentration of Compound I.

In another embodiment, the individual in need thereof is administered Compound I, or a pharmaceutically acceptable salt thereof, in an amount that provides in the plasma of the individual following the administration to the individual of Compound I, or a pharmaceutically acceptable salt thereof, (a) a Cmax of Compound I that is greater than or equal to 0.2 nM and less than or equal to 10 nM, and (b) a mean unbound concentration of Compound I that is greater than or equal to 0.2 nM and less than or equal to 10 nM for at least 20 hours. In another embodiment, the individual in need thereof is administered Compound I, or a pharmaceutically acceptable salt thereof, in an amount that provides a Cmax of Compound I in the plasma of the individual that is greater than or equal to 0.2 nM and less than or equal to 9 nM, or greater than or equal to 0.2 nM and less than or equal to 8.5 nM, or greater than or equal to 0.2 nM and less than or equal to 8 nM, or greater than or equal to 0.2 nM and less than or equal to 7.5 nM, or greater than or equal to 0.2 nM and less than or equal to 7 nM, or greater than or equal to 0.2 nM and less than or equal to 6.5 nM, or greater than or equal to 0.2 nM and less than or equal to 6 nM, or greater than or equal to 0.2 nM and less than or equal to 5.5 nM, or greater than or equal to 0.2 nM and less than or equal to 5 nM, or greater than or equal to 0.2 nM and less than or equal to 4.5 nM, or greater than or equal to 0.2 nM and less than or equal to 4 nM, or greater than or equal to 0.2 nM and less than or equal to 3.5 nM, greater than or equal to 0.2 nM and less than or equal to 3 nM, or greater than or equal to 0.2 nM and less than or equal to 2.75 nM following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 20 hours following the administration to the individual of Compound I, or a pharmaceutically acceptable salt thereof. In some embodiments, the salt of Compound I is the malonate salt. It is understood that the disclosure herein contemplates any combinations of the values disclosed for Cmax with any values disclosed herein for mean unbound concentration of Compound I.

In another embodiment, the individual in need thereof is administered Compound I, or a pharmaceutically acceptable salt thereof, in an amount that provides in the plasma of the individual following the administration to the individual of Compound I, or a pharmaceutically acceptable salt thereof, (a) a Cmax of Compound I that is greater than or equal to 0.2 nM and less than or equal to 10 nM, and (b) a mean unbound concentration of Compound I that is greater than or equal to 0.2 nM and less than or equal to 10 nM for at least 24 hours. In another embodiment, the individual in need thereof is administered Compound I, or a pharmaceutically acceptable salt thereof, in an amount that provides a Cmax of Compound I in the plasma of the individual that is greater than or equal to 0.2 nM and less than or equal to 9 nM, or greater than or equal to 0.2 nM and less than or equal to 8.5 nM, or greater than or equal to 0.2 nM and less than or equal to 8 nM, or greater than or equal to 0.2 nM and less than or equal to 7.5 nM, or greater than or equal to 0.2 nM and less than or equal to 7 nM, or greater than or equal to 0.2 nM and less than or equal to 6.5 nM, or greater than or equal to 0.2 nM and less than or equal to 6 nM, or greater than or equal to 0.2 nM and less than or equal to 5.5 nM, or greater than or equal to 0.2 nM and less than or equal to 5 nM, or greater than or equal to 0.2 nM and less than or equal to 4.5 nM, or greater than or equal to 0.2 nM and less than or equal to 4 nM, or greater than or equal to 0.2 nM and less than or equal to 3.5 nM, greater than or equal to 0.2 nM and less than or equal to 3 nM, or greater than or equal to 0.2 nM and less than or equal to 2.75 nM following the administration to the individual of Compound I, or a pharmaceutically acceptable salt thereof. In some embodiments, the individual in need thereof is administered Compound I, or a pharmaceutically acceptable salt thereof, in an amount that provides a mean unbound concentration of Compound I in the plasma of the individual that is greater than or equal to 0.2 nM, or 0.25 nM, or 0.3 nM, or 0.35 nM, or 0.4 nM, or 0.45 nM, or 0.5 nM, or 0.55 nM, or 0.6 nM, or 0.65 nM, or 0.7 nM, 0.8 nM, or 0.9 nM, or 1 nM, or 1.25 nM, or 1.5 nM, or 1.75 nM, or 2 nM for at least 24 hours following the administration to the individual of Compound I, or a pharmaceutically acceptable salt thereof. In some embodiments, the salt of Compound I is the malonate salt. It is understood that the disclosure herein contemplates any combinations of the values disclosed for Cmax with any values disclosed herein for mean unbound concentration of Compound I.

In another embodiment are provided the methods disclosed herein, wherein the individual achieves stable disease, a partial response, or a complete response in the individual's target tumors in accordance with RECIST 1.1 following administration to the individual of Compound I, or a pharmaceutically acceptable salt thereof. In some embodiments, the salt of Compound I is a malonate salt. In some embodiments, the response in accordance with RECIST 1.1 is a partial response or a complete response. In some embodiments, the response in accordance with RECIST 1.1 is a partial response. In some embodiments, the response in accordance with RECIST 1.1 is a complete response.

In another embodiment are provided the methods disclosed herein, wherein the individual does not experience any Grade 3 or Grade 4 treatment related adverse events following administration of Compound I, or a pharmaceutically acceptable salt thereof, to the individual. In some embodiments the salt of Compound I is a malonate salt.

Those of ordinary skill in the art will understand that with respect to Compound I, or a pharmaceutically acceptable salt thereof, as described herein the particular pharmaceutical formulation, the dosage, and the number of doses given per day to an individual requiring such treatment, are all choices within the knowledge of one of ordinary skill in the art and can be determined without undue experimentation.

Dosages of compositions described herein can be determined by any suitable method. Maximum tolerated doses (MTD) and maximum response doses (MRD) of a salt of Compound I, or a pharmaceutically acceptable salt thereof, as described herein can be determined via established animal and human experimental protocols as well as in the examples described herein. For example, toxicity and therapeutic efficacy of Compound I, or a pharmaceutically acceptable salt thereof, as described herein can be determined by standard pharmaceutical procedures in cell cultures or experimental animals, including, but not limited to, for determining the LD50 (the dose lethal to 50% of the population) and the ED50 (the dose therapeutically effective in 50% of the population). The dose ratio between the toxic and therapeutic effects is the therapeutic index and it can be expressed as the molar ratio between LD50 and ED50. The data obtained from cell culture assays and animal studies can be used in formulating a range of dosage for use in human. The dosage of Compound I, or a pharmaceutically acceptable salt thereof, as described herein lies preferably within a range of circulating concentrations that include the ED50 with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of Compound I, or a pharmaceutically acceptable salt thereof, as described herein comprising a formulation that corresponds to such an amount varies depending upon factors such as the particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is 56 days. In some embodiments, a treatment cycle lasts one, two, three, or four weeks. In some embodiments, a treatment cycle lasts three weeks. In some embodiments, a treatment cycle lasts four weeks. The number of treatment doses scheduled within each cycle also varies depending on the drugs being given.

In one aspect, compositions comprising Compound I, or a pharmaceutically acceptable salt thereof, as described herein are used for the treatment of cancer in a subject. In one embodiment, such compositions are in the form of suitable dosage forms. Suitable dosage forms include, for example, liquids, suspensions, powders for reconstitution, tablets, pills, sachets, or capsules of hard or soft gelatin (See, e.g., Remington: The Science and Practice of Pharmacy (Gennaro, 21st Ed. Mack Pub. Co., Easton, PA (2005)).

Compound I, or a pharmaceutically acceptable salt thereof, as described herein may be formulated into pharmaceutical compositions as described below in any pharmaceutical form recognizable to the skilled artisan as being suitable. Pharmaceutical compositions of the disclosure comprise a therapeutically effective amount of at least one salt of Compound I of the present disclosure and an inert, pharmaceutically acceptable carrier or diluent.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, Compound I, or a pharmaceutically acceptable salt thereof, as described herein may be dissolved in an aqueous solution of an organic or inorganic acid, such as a 0.3 M solution of succinic acid or citric acid. If a soluble salt form is not available, the agent may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, a salt of Compound I as described herein is dissolved in DMSO and diluted with water. The composition may also be in the form of a solution of the salt form of the active ingredient in an appropriate aqueous vehicle such as water or isotonic saline or dextrose solution.

Proper formulation is dependent upon the route of administration selected. For injection, the agents of Compound I, or a pharmaceutically acceptable salt thereof, of the present disclosure may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, Compound I, or a pharmaceutically acceptable salt thereof, as described herein can be formulated by combining the active compounds with pharmaceutically acceptable carriers known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active ingredient.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, Compound I, or a pharmaceutically acceptable salt thereof, for use according to the present disclosure may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the salt of Compound I as described herein and a suitable powder base such as lactose or starch.

Compound I, or a pharmaceutically acceptable salt thereof, as described herein may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active ingredients may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the salts of Compound I as described herein to allow for the preparation of highly concentrated solutions.

Alternatively, the active ingredient may be in powder form for constitution with a suitable vehicle, e.g. sterile pyrogen-free water, before use.

In addition to the formulations described above, Compound I, or a pharmaceutically acceptable salt thereof, may also be formulated as a depot preparation. Such long-acting formulations may be administered by implantation (for example, subcutaneously or intramuscularly) or by intramuscular injection. Thus, for example, the salt of Compound I as described herein may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire, Capryol, Labrafil, Labrasol, Lauroglycol, Plurol, Peceol, Transcutol, and the like may be used.

Further, the pharmaceutical composition may be incorporated into a skin patch for delivery of the drug directly onto the skin.

Additionally, the pharmaceutically acceptable formulations to be used according to the methods disclosed herein may comprise Compound I, or a pharmaceutically acceptable salt thereof, as described herein in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

### Articles of Manufacture and Kits

The kit or article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active ingredient in the composition is a) Compound I, or a pharmaceutically acceptable salt thereof, as described herein; and/or b) one or more additional anticancer agents such as any of the anticancer agents described herein. The label or package insert indicates that the composition is used for treating cancer (e.g., a particular cancer) in an individual. The label or package insert will further comprise instructions for administering the composition to the individual (e.g., according to any of the methods described herein). Articles of manufacture and kits comprising combination therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating cancer.

Additionally, the kit or article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

For example, the kit or article of manufacture comprises a) a composition comprising a composition comprising Compound I, or a pharmaceutically acceptable salt thereof, as described herein and b) instructions for administering a composition comprising Compound I, or a pharmaceutically acceptable salt thereof, as described herein to an individual for treatment of a cancer, optionally in combination with one or more additional anticancer agents. In some embodiments, the kit or article of manufacture comprises a) a composition comprising Compound I, or a pharmaceutically acceptable salt thereof, as described herein b) one or more additional anticancer agents such as any of the anticancer agents described herein, and c) instructions for administering Compound I, or a pharmaceutically acceptable salt thereof, as described herein and the one or more additional anticancer agents to an individual for treatment of a cancer.

The composition comprising Compound I, or a pharmaceutically acceptable salt thereof, as described herein and the one or more additional anticancer agents can be present in separate containers or in a single container. For example, the kit may comprise one distinct composition or two or more compositions wherein one composition comprises a composition comprising Compound I, or a pharmaceutically acceptable salt thereof, as described herein and another composition comprises the one or more additional anticancer agents.

The kits or articles of manufacture of the application are in suitable packaging. Suitable packaging includes, but is not limited to, vials, bottles, jars, flexible packaging (e.g., sealed Mylar or plastic bags), and the like. Kits or articles of manufacture may optionally provide additional components such as buffers and interpretative information. The present application thus also provides articles of manufacture, which include vials (such as sealed vials), bottles, jars, flexible packaging, and the like.

The instructions relating to the use of Compound I, or a pharmaceutically acceptable salt thereof, as described herein and one or more additional anticancer agents generally include information as to dosage, dosing schedule, and route of administration for the intended treatment. The containers may be unit doses, bulk packages (e.g., multi-dose packages) or sub-unit doses. For example, kits may be provided that contain sufficient dosages of a composition comprising Compound I, or a pharmaceutically acceptable salt thereof, as described herein and one or more additional anticancer agents as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, 12 months, 24 months or more. Kits may also include multiple unit doses of Compound I, or a pharmaceutically acceptable salt thereof, as described herein and the one or more additional anticancer agents and instructions for use, packaged in quantities sufficient for storage and use in pharmacies, for example, hospital pharmacies and compounding pharmacies.

Compound I may be prepared using the methods outlined in Scheme 1 below.

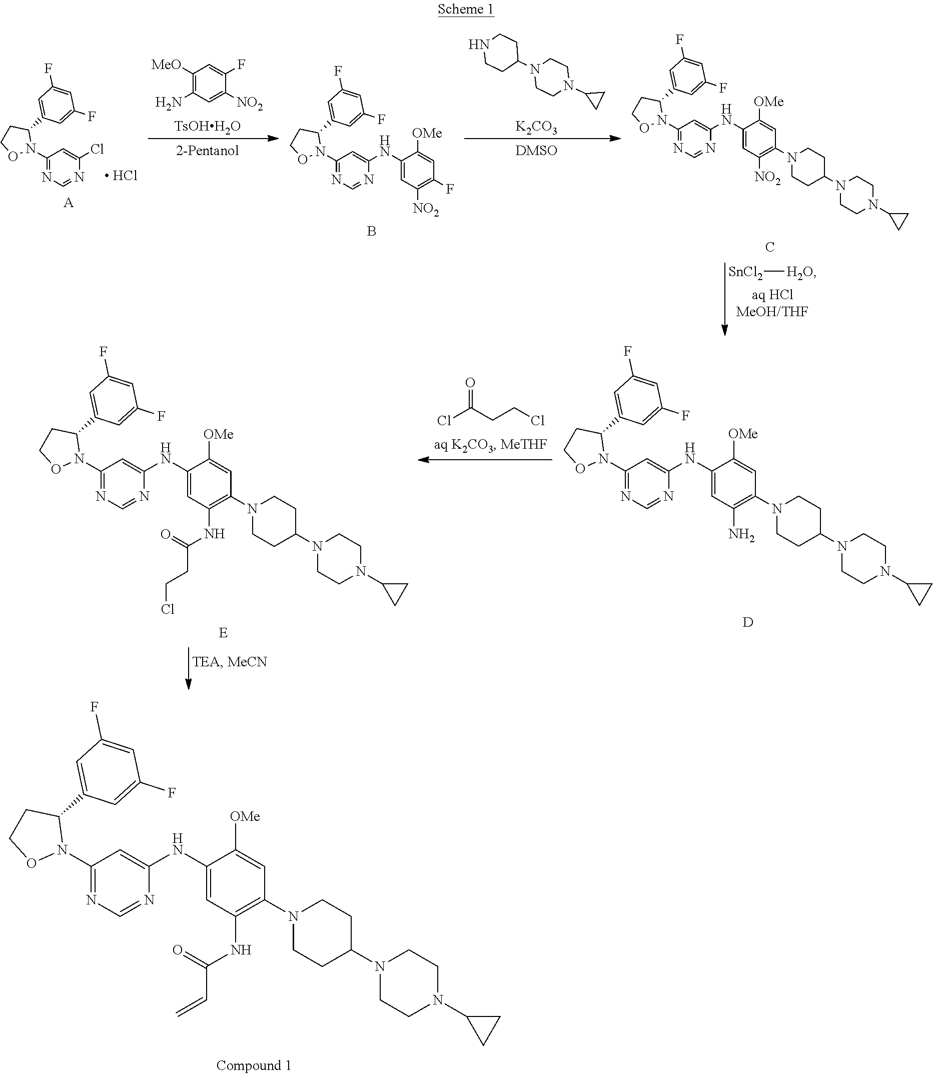

(R)-2-(6-chloropyrimidin-4-yl)-3-(3,5-difluorophenyl)isoxazolidine (Compound A) may be prepared by methods analogous to those described in United States Patent Application Publication No. U.S. 20220073505 A1, which is hereby incorporated by reference for that purpose. (R)-2-(6-chloropyrimidin-4-yl)-3-(3,5-difluorophenyl)isoxazolidine may be allowed to react with 4-fluoro-2-methoxy-5-nitroaniline to provide (R)-6-(3-(3,5-difluorophenyl)isoxazolidin-2-yl)-N-(4-fluoro-2-methoxy-5-nitrophenyl)pyrimidin-4-amine (Compound B). The reaction of Compound A and 4-fluoro-2-methoxy-5-nitroaniline may be conducted in a protic solvent, or a mixture of protic solvents, optionally in the presence water, and in the presence of an acid. For example, the Compound A may be allowed to react with 4-fluoro-2-methoxy-5-nitroaniline in an alcohol, such as 2-pentanol, and in the precent of para-toluenesulfonic acid, and at a temperature of from about 0° C. to about 100° C. to provide Compound B. 4-fluoro-2-methoxy-5-nitroaniline may be purchased or synthesized by methods known to those having ordinary skill in the art. In turn, Compound B may be allowed to react with 1-cyclopropyl-4-(piperidin-4-yl)piperazine in an aprotic solvent and in the presence of a base to afford (R)—N-(4-(4-(4-cyclopropylpiperazin-1-yl)piperidin-1-yl)-2-methoxy-5-nitrophenyl)-6-(3-(3,5-difluorophenyl)isoxazolidin-2-yl)pyrimidin-4-amine (Compound C). For example, Compound B may be allowed to react with 1-cyclopropyl-4-(piperidin-4-yl)piperazine in dimethylsulfoxide and in the presence of potassium carbonate, and at a temperature of from about 0° C. to about 100° C. to afford Compound C. The compound 1-cyclopropyl-4-(piperidin-4-yl)piperazine may be prepared using methods known to those having ordinary skill in the art and methods similar to those disclosed in United States Patent Application Publication No. U.S. 20220073505 A1. In turn, Compound C may be allowed to react with a reducing agent and in the presence of a protic solvent, an aprotic solvent, or a mixture of a protic and an aprotic solvent depending on the reducing agent used, to afford (R)-4-(4-(4-cyclopropylpiperazin-1-yl)piperidin-1-yl)-N1-(6-(3-(3,5-difluorophenyl)isoxazolidin-2-yl)pyrimidin-4-yl)-6-methoxybenzene-1,3-diamine (Compound D). For example, Compound C may be allowed to react with tin (II) dichloride as a reducing agent in a mixture of methanol and tetrahydrofuran as solvent, and a temperature of from about 0° C. to about 100° C. to afford Compound D. In turn, Compound D may be allowed to react with a reagent capable of selectively reacting with the primary amino group in Compound D to afford (R)-3-chloro-N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)propenamide (Compound E). For example, Compound D may be allowed to react with 3-chloropropanoyl chloride in an aprotic solvent, such as 2-methyltetrahydrofuran, and in the presence of a base, such as potassium carbonate, and at a temperature of from about 0° C. to about 100° C. to afford Compound E. Compound E may be isolated, or may be used in the next reaction without further purification. Compound E may be allowed to react with a base capable of causing an elimination reaction to afford (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide (Compound I). For example, Compound E may be allowed to react with a trialkylamine base, such as triethylamine (TEA), in the presence of an aprotic solvent, such as acetonitrile, and at a temperature from about 0° C. to about 100° C., to afford Compound I. Compound I may be isolated as the free base, or the free base may be used without further purification in a reaction to afford a suitable salt form, such as a malonate or glycolate salt as further described herein.

### ENUMERATED EMBODIMENTS

Various embodiments are contemplated herein. For example, in Embodiment 1, provided is a method of treating cancer in an individual in need thereof, wherein the cancer in the individual has been determined to comprise one or more atypical epidermal growth factor receptor (EGFR) mutations, comprising administering to the individual (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

Embodiment 2: The method of Embodiment 1, wherein the one or more atypical epidermal growth factor receptor (EGFR) mutations in the cancer of the individual comprises one or more (a) P-loop and alpha C-helix compressing (PACC) EGFR mutations, (b) Exon18 mutations, (c) Exon19 mutations, (d) Exon20 point mutations, (e) Exon21 mutations, (f) mutations in the extracellular domain of EGFR, (g) mutations in the transmembrane domain of EGFR, and (h) Exon20 insertion mutations, provided that the Exon20 insertion mutations do not include EGFR_Exon20insNPH, EGFR_Exon20insSVD, EGFR_Exon20insFQEA, EGFR_Exon20insH, and EGFR_Exon20insASV, EGFR_Exon20insYVMA.

Embodiment 3: The method of Embodiment 1, wherein the more atypical epidermal growth factor receptor (EGFR) mutations in the cancer of the individual is selected from the group consisting of EGFR_A702T, EGFR_E709A, EGFR_E709A G719A, EGFR_E709A G719S, EGFR_E709K, EGFR_E709K G719S, EGFR_E709_T710delinsD, EGFR_E709_T710del insD S22R, EGFR_L718Q, EGFR L718Q L858R, EGFR_L718V, EGFR_L718V L858R, EGFR_G719A, EGFR_G719A D761Y, EGFR_G719A L861Q, EGFR_G719A R776C, EGFR_G719A S768L, EGFR_G719C, EGFR_G719C S768L, EGFR_G719S, EGFR_G719S L861Q, EGFR_T725M, EGFR_G719S S768L, EGFR_S720P, EGFR_E736K, EGFR_G724S, EGFR_G724S Exl9del, EGFR_G724S L858R, EGFR_I740dupIPVAK, EGFR_E746_A750del A647T, EGFR_E746_A750del G724S, EGFR_E746_A750del S768L, EGFR_E746_A750del R675W, EGFR_E746_T751del insV, EGFR_E746_T751del insV S768C, EGFR_T751_I759 delinsN, EGFR_L747P, EGFR_L747S, EGFR_L747S V774M, EGFR_L747S L858R, EGFR_L747_S752del A755D, EGFR_A750_I759del insPN, EGFR S752 I759del V769M, EGFR_K757M L858R, EGFR_K757R, EGFR_D761N, EGFR_A767dupASV, EGFR_S768C, EGFR_S768I, EGFR_S768I V769L, EGFR_S768I V774M, EGFR_S768I L858R, EGFR_S768I L861Q, EGFR_S768dupSVD, EGFR_V769L, EGFR_V769M, EGFR_N771G, EGFR_H773dup, EGFR_V774M, EGFR_R776C, EGFR_R776H, EGFR_G779F, EGFR_L792H, EGFR Exl9del L792H, EGFR G796S, EGFR V774M, EGFR S784F, EGFR Exl9del G796S, EGFR_L833V, EGFR_V834L, EGFR_T854I, EGFR_Exl9del T854L, EGFR_L858R L792H, EGFR_L858R C797S, EGFR_L858R T854S, EGFR_L861Q, EGFR_L861R, EGFR_S811F, EGFR_A763insFQEA, EGFR_A763insLQEA, EGFR_E746_A750delL41W, EGFR E746_A750delR451H, EGFR_K754E, EGFR L747_E749del, EGFR_A750P, EGFR_L747_T751del, and EGFR_L833F.

Embodiment 4: The method of any one of Embodiments 1 to 3, wherein the cancer in the individual has been determined to further comprise one or more classical EGFR mutations.

Embodiment 5: The method of any one of Embodiments 1 to 3, wherein the cancer in the individual has been determined to further comprise one or more EGFR mutation selected from T790M, L858R, one or more exon 19 deletions, and one or more exon 20 insertion mutations.

Embodiment 6: The method of any one of Embodiments 1 to 3, wherein the cancer in the individual has been determined to further comprise one or more mutations in EGFR selected from the group consisting of G309A, G309E, S310F, R678Q, R678Q and L755W, L755S, L755W, 1767M, D769H, D769Y, V777L, Y835F, V842I, R896C, G1201V, del.755-759, EGFR Del19/T790M, EGFR L858R/T790M, EGFR L858R, EGFR del 19, EGFR_del746-750, EGFR L858R/C797S, EGFR del19/C797S, EGFR Exon20 ins NPH, EGFR Exon20 ins SVD, EGFR Exon20 ins FQEA, EGFR Exon20 ins H, and EGFR Exon20 ins ASV.

Embodiment 7: The method of any one of Embodiments 1 to 6, wherein the (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is administered to the individual in the form of a salt.

Embodiment 8: The method of Embodiment 7, wherein the (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is administered to the individual in the form of a fumarate, hemi-fumarate, glycolate or malonate salt.

Embodiment 9: The method of Embodiment 8, wherein the (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is administered to the individual in the form of a fumarate salt.

Embodiment 10: The method of Embodiment 8, wherein the (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is administered to the individual in the form of a hemi-fumarate salt.

Embodiment 11: The method of Embodiment 8, wherein the (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is administered to the individual in the form of a glycolate salt.

Embodiment 12: The method of Embodiment 8, wherein the (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is administered to the individual in the form of a malonate salt.

Embodiment 13: The method of Embodiment 12, wherein the malonate salt is a hemi-malonate salt.

Embodiment 14: The method of Embodiment 12 or Embodiment 13, wherein the salt is in a crystalline form.

Embodiment 15: The method of Embodiment 14, wherein the crystalline form of the salt maintains at least 95% of the crystalline form following storage in an open container for at least 7 days at 40° C. and 75% relative humidity, and wherein the amount of the crystalline form of the salt is measured by XRPD.

Embodiment 16: The method of Embodiment 14, wherein the crystalline form of the salt maintains at least 95% of the crystalline form following storage in an open container for at least 14 days at 40° C. and 75% relative humidity, and wherein the amount of the crystalline form of the salt is measured by XRPD.

Embodiment 17: The method of Embodiment 14, wherein the crystalline form of the salt maintains at least 95% of the crystalline form following storage in a closed container for at least 7 days at 40° C. and 75% relative humidity, and wherein the amount of the crystalline form of the salt is measured by XRPD.

Embodiment 18: The method of Embodiment 14, wherein the crystalline form of the salt maintains at least 95% of the crystalline form following storage in a closed container for at least 14 days at 40° C. and 75% relative humidity, and wherein the amount of the crystalline form of the salt is measured by XRPD.

Embodiment 19: The method of any one of Embodiments 12 to 18, wherein the malonate salt has a solubility in an aqueous solution having a pH of about 4.5 and at a temperature of about 37° C., calculated as an amount of the free base of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, of greater than about 2 mg/mL.

Embodiment 20: The method of any one of Embodiments 12 to 19, wherein the malonate salt has a solubility in an aqueous solution having a pH of about 2.9 and at a temperature of about 37° C., calculated as an amount of the free base of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, of greater than about 2 mg/mL.

Embodiment 21: The method of any one of Embodiments 14 to 20, wherein the malonate salt exhibits an XRPD pattern comprising a peak at 5.9±0.2 degrees 2-theta.

Embodiment 22: The method of Embodiment 21, wherein the malonate salt further comprises a peak in the XRPD pattern at 6.8±0.2 degrees 2-theta.

Embodiment 23: The method of Embodiment 22, wherein the malonate salt further comprises a peak in the XRPD pattern at 16.0±0.2 degrees 2-theta.

Embodiment 24: The method of Embodiment 23, wherein the malonate salt further comprises peaks in the XRPD pattern at 17.1±0.2, 19.0±0.2, and 21.6±0.2 degrees 2-theta.

Embodiment 25: The method of Embodiment 24, wherein the malonate salt further comprises peaks in the XRPD pattern at 12.2±0.2, 20.5±0.2, and 23.7±0.2 degrees 2-theta.

Embodiment 26: The method of any one of Embodiments 12 to 25, wherein the malonate salt exhibits a differential scanning calorimetry trace comprising a peak of from about 147° C. to about 151° C.

Embodiment 27: The method of any of Embodiments 12 or 13, wherein the malonate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is in crystalline form, and wherein the crystalline form exhibits (a) an XRPD pattern comprising a peak at 5.9±0.2 degrees 2-theta, and (b) a differential scanning calorimetry trace comprising a peak of from about 147° C. to about 151° C.

Embodiment 28: The method of Embodiment 27, wherein the malonate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form further comprises a peak in the XRPD pattern at 6.8±0.2 degrees 2-theta.

Embodiment 29: The method of Embodiment 28, wherein the malonate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form further comprises a peak in the XRPD pattern at 16.0±0.2 degrees 2-theta.

Embodiment 30: The method of Embodiment 29, wherein the malonate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form further comprises peaks in the XRPD pattern at 17.1±0.2, 19.0±0.2, and 21.6±0.2 degrees 2-theta.

Embodiment 31: The method of Embodiment 30, wherein the malonate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form further comprises peaks in the XRPD pattern 12.2±0.2, 20.5±0.2, and 23.7±0.2 degrees 2-theta.

Embodiment 32: The method of any one of Embodiments 27 to 31, wherein the malonate salt has a solubility in an aqueous solution having a pH of about 4.5 and at a temperature of about 37° C., calculated as an amount of the free base of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, of greater than about 2 mg/mL.

Embodiment 33: The method of any one of Embodiments 27 to 32, wherein the malonate salt has a solubility in an aqueous solution having a pH of about 2.9 and at a temperature of about 37° C., calculated as an amount of the free base of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, of greater than about 2 mg/mL.

Embodiment 34: The method of any one of Embodiments 1 to 8, wherein the salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is a glycolate salt.

Embodiment 35: The method of Embodiment 34, wherein the glycolate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is a hemi-glycolate salt.

Embodiment 36: The method of Embodiment 34 or Embodiment 35, wherein the glycolate salt is in a crystalline form.

Embodiment 37: The method of Embodiment 36, wherein the glycolate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form exhibits an XRPD pattern comprising a peak at 14.1±0.2 degrees 2-theta.

Embodiment 38: The method of Embodiment 37, wherein the glycolate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form further comprises a peak in the XRPD pattern at 19.4±0.2 degrees 2-theta.

Embodiment 39: The method of Embodiment 38, wherein the glycolate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form further comprises a peak in the XRPD pattern 20.1±5.2 degrees 2-theta.

Embodiment 40: The method of Embodiment 39, wherein the glycolate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form further comprises peaks in the XRPD pattern at 5.4±0.2, 9.6±0.2, 13.0±0.2, 15.5±0.2, and 22.4±0.2 degrees 2-theta.

Embodiment 41: The method of any one of Embodiments 34 to 40, wherein the glycolate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide salt exhibits a differential scanning calorimetry trace comprising a peak of from about 162° C. to about 165° C.

Embodiment 42: The method of any one of Embodiments 34 or 35, wherein the glycolate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is in crystalline form, and wherein the crystalline form exhibits (a) an XRPD pattern comprising a peak at 14.1±0.2 degrees 2-theta, and (b) a differential scanning calorimetry trace comprising a peak of from about 162° C. to about 165° C.

Embodiment 43: The method of Embodiment 42, wherein the glycolate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form further comprises a peak in the XRPD pattern at 20.1±0.2 degrees 2-theta.

Embodiment 44: The method of Embodiment 43, wherein the glycolate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in crystalline form further comprises peaks in the XRPD pattern at 5.4±0.2 9.6±0.2, 13.0±0.2, 14.1±0.2, 15.5±0.2, and 22.4±0.2 degrees 2-theta.

Embodiment 45: The method of any one of Embodiments 1 to 44, wherein the cancer in the individual is selected from the group consisting of pseudomyxoma, intrahepatic cholangiocarcinoma, hepatoblastoma, liver cancer, thyroid cancer, colon cancer, testicular cancer, myelodysplastic syndrome, glioblastoma, oral cancer, lip cancer, mycosis fungoides, acute myeloid leukemia, acute lymphocytic leukemia, basal cell carcinoma, ovarian epithelial cancer, ovarian germ cell cancer, male breast cancer, brain cancer, pituitary adenomas, multiple myeloma, gallbladder cancer, biliary tract cancer, colorectal cancer, chronic myelogenous leukemia, chronic lymphocytic leukemia, retinoblastoma, choroidal melanoma, ampulla of vater cancer, bladder cancer, peritoneal cancer, parathyroid cancer, adrenal cancer, nasal and paranasal sinus cancer, non-small cell lung cancer, tongue cancer, astrocytoma, small cell lung cancer, childhood brain cancer, pediatric lymphoma, pediatric leukemia, small intestine cancer, meningioma, esophageal cancer, glioma, renal carcinoma, kidney cancer, heart cancer, duodenal cancer, malignant soft tissue cancer, malignant bone cancer, malignant lymphoma, malignant mesothelioma, malignant melanoma, eye cancer, vulvar cancer, ureter cancer, urethral cancer, cancer of unknown primary site, gastric lymphoma, gastric cancer, gastric carcinoid tumors, gastrointestinal stromal tumors, Wilms cancer, breast cancer, sarcoma, penile cancer, pharyngeal cancer, gestational trophoblastic disease, cervical cancer, endometrial cancer, uterine sarcoma, prostate cancer, metastatic bone cancer, metastatic brain cancer, mediastinal cancer, rectal cancer, rectal carcinoid tumors, vaginal cancer, spinal carcinoma, acoustic neuroma, pancreatic cancer, salivary gland cancer, Kaposi's sarcoma, Paget's disease, tonsillar cancer, squamous cell carcinoma, lung adenocarcinoma, lung cancer, lung squamous cell carcinoma, skin cancer, anal cancer, rhabdomyosarcoma, laryngeal cancer, pleural cancer, blood cancer, and thymic carcinoma.

Embodiment 46: The method of Embodiment 45, wherein the cancer in the individual is selected from the group consisting of metastatic brain cancer, breast cancer, and non-small cell lung cancer.

Embodiment 47: The method of Embodiment 46, wherein the cancer in the individual is metastatic brain cancer.

Embodiment 48: The method of Embodiment 46, wherein the cancer in the individual is breast cancer.

Embodiment 49: The method of Embodiment 46, wherein the cancer in the individual is non-small cell lung cancer.

Embodiment 50: The method of any one of Embodiments 1 to 49, wherein the cancer is a locally advanced cancer.

Embodiment 51: The method of any one of Embodiments 1 to 50, wherein the cancer is unresectable.

Embodiment 52: The method of any one of Embodiments 1 to 51, wherein the cancer comprises one or more central nervous system (CNS) metastases (e.g., brain metastases).

Embodiment 53: The method of any one of Embodiments 1 to 52, wherein the individual has not received one or more prior therapy for treatment of the cancer prior to administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

Embodiment 54: The method of any one of Embodiments 1 to 52, wherein the individual has received one or more prior therapy for treatment of the cancer prior to administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

Embodiment 55: The method of any one of Embodiments 1 to 54, wherein the method further comprises administering to the individual in need thereof one or more additional anticancer agents.

Embodiment 56: The method of Embodiment 55, wherein the one or more additional anticancer agents comprises one or more agents selected from HER2 inhibitors, HER2-CD3 bispecific antibodies, HER2-immune targeting bispecific antibodies, anti-HER2 chimeric antigen receptor (CAR) T cells, anti-HER2 chimeric antigen receptor (CAR) cytotoxic T-lymphocytes (CTLs), anti-HER2 chimeric antigen receptor (CAR) natural killer (NK) cells, anti-HER2 chimeric antigen receptor (CAR) cytokine-induced killer (CIK) cells, epidermal growth factor receptor (EGFR) inhibitors, poly-ADP-ribose polymerase (PARP) inhibitors, PD-1 inhibitors, PD-L1 inhibitors, Phosphoinositide 3-kinase (PI3K) inhibitors, and chemotherapeutic agents.

Embodiment 57: The method of Embodiment 55, wherein the one or more additional anticancer agents are selected from the group consisting of trastuzumab, trastuzumab and hyaluronidase, capecitabine, trastuzumab and capecitabine, tucatinib, lapatinib, neratinib, dacomitinib, pertuzumab, margetuximab, trastuzumab emtansine, trastuzumab deruxtecan, ZW49 (Zymeworks), A166 (Klaus Pharma), ARX788 (Ambrx), RC48-ADC (RemeGen), vic-trastuzumab duocarmazine, zanidatamab (ZW25), zenocutuzamab (MCLA-128), ISB 1302, afatanib, poziotinib, pyrotinib, mobocertinib (TAK788), and BDTX-189, eriotinib, osimertinib, gefitinib, cetuximab, panitumumab, necitumumab, vandetanib, afatinib, brigatinib, icotinib, niraparib, olaparib, talazoparib, rucaparib, veliparib, iniparib, pamiparib (BGB-290), CEP-9722, E7016, pembrolizumab, nivolumab, cemiplimab, JTX-4014, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, atezolizumab, avelumab, durvalumab, KN035, CK-301, AUNP12, CA-170, and BMS-986189.

Embodiment 58: The method of Embodiment 55, wherein the one or more additional anticancer agents are selected from antibody-drug conjugates.

Embodiment 59: The method of Embodiment 58, wherein the antibody-drug conjugates are selected from trastuzumab emtansine and trastuzumab deruxtecan.

Embodiment 60: The method of Embodiment 55, wherein the one or more additional anticancer agents comprises one or more chemotherapeutic agents.

Embodiment 61: The method of Embodiment 60, wherein the one or more chemotherapeutic agents is selected from the group consisting of doxorubicin, docetaxel, pemetrexed, paclitaxel, carboplatin, cisplatin, capecitabine, gemcitabine, vinorelbine, temozolomide, irinotecan, oxiplatin, and eribulin.

Embodiment 62: The method of Embodiment 57, wherein the one or more additional anticancer agents comprise trastuzumab and capecitabine.

Embodiment 63: The method of any one of Embodiments 1 to 62, wherein the method further comprises treating the individual in need thereof with radiation.

Embodiment 64: The method of any one of Embodiments 1 to 63, wherein the cancer does not comprise a mutation in phosphatidylinositol-4,5-bisphosphate 3-kinase catalytic subunit alpha (PIK3CA) protein.

Embodiment 65: The method of Embodiment 64, wherein the mutation in the PIK3CA protein comprises one or more mutations at histidine 1047.

Embodiment 66: The method of Embodiment 65, wherein the mutation at histidine 1047 in the PIK3CA protein is selected from H1047L and H1047R.

Embodiment 67: The method of any one of Embodiments 1 to 66, wherein the (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, is administered to the individual in need thereof orally, parentally, intravenously, subcutaneously, or intracerebrally.

Embodiment 68: The method of any one of Embodiments 1 to 67, wherein the individual is a human.

Embodiment 69: The method of any one of Embodiments 1 to 68, wherein the cancer has been determined by use of an FDA-approved test to comprise one or more atypical epidermal growth factor receptor (EGFR) mutations.

Embodiment 70: The method of any one of Embodiments 1 to 69, wherein in the cancer in the individual has been determined by use of any nucleic acid-based diagnostic testing method to comprise one or more atypical epidermal growth factor receptor (EGFR) mutations.

Embodiment 71: The method of any one of Embodiments 1 to 46 and 50 to 70, wherein in the cancer in the individual is non-small cell lung cancer and the cancer has been determined by use of any nucleic acid-based diagnostic testing method to comprise one or more atypical epidermal growth factor receptor (EGFR) mutations.

Embodiment 72: The method of any one of Embodiments 1 to 46 and 50 to 71, wherein in the cancer in the individual is locally advanced or metastatic non-small cell lung cancer with one or more atypical epidermal growth factor receptor (EGFR) mutations as detected by an FDA-approved test, and wherein the cancer has progressed on or after platinum-based chemotherapy.

Embodiment 73: The method of any one of Embodiments 1 to 46 and 50 to 72, wherein in the cancer in the individual is locally advanced or metastatic non-small cell lung cancer with one or more atypical epidermal growth factor receptor (EGFR) mutations, as detected by an FDA-approved test, and wherein the cancer has progressed following prior EGFR therapy.

Embodiment 74: The method of any one of Embodiments 1 to 46 and 50 to 73, wherein the cancer is non-small cell lung cancer and the individual has not received prior therapy for treatment of the cancer with an EGFR inhibitor prior to administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

Embodiment 75: The method of any one of Embodiments 1 to 74, wherein the individual is EGFR inhibitor naïve prior to administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

Embodiment 76: The method of any one of Embodiments 1 to 46 and 50 to 75, wherein the cancer is non-small cell lung cancer and the individual is EGFR inhibitor naive to administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

Embodiment 77: The method of any one of Embodiments 1 to 73, wherein the individual has received one or more prior EGFR inhibitor therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

Embodiment 78: The method of any one of Embodiments 1 to 46, 50 to 73, and 77, wherein the cancer is non-small cell lung cancer and the individual has received one or more prior EGFR inhibitor therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

Embodiment 79: The method of any one of Embodiments 1 to 73, 77, and 78, wherein the individual has received one or more prior EGFR inhibitor therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, and wherein the individual is further administered a bispecific EGF receptor-directed and MET receptor-directed antibody.

Embodiment 80: The method of any one of Embodiments 1 to 73 and 77 to 79, wherein the individual has received one or more prior EGFR inhibitor therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, and wherein the cancer is non-small cell lung cancer and the individual is further administered a bispecific EGF receptor-directed and MET receptor-directed antibody.

Embodiment 81: The method of any one of Embodiments 78 and 79, wherein the bispecific EGF receptor-directed and MET receptor-directed antibody is amivantamab.

Embodiment 82: The method of any one of Embodiments 1 to 73 and 77 to 81, wherein (a) the cancer is non-small cell lung cancer and (b) the individual has received one or more prior EGFR inhibitor therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, and wherein individual is further administered amivantamab.

Embodiment 83: The method of any one of Embodiments 1 to 73, 77, and 78, wherein the individual has received one or more prior therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, and wherein the one or more prior therapies comprise an EGFR inhibitor and a bispecific EGF receptor-directed and MET receptor-directed antibody.

Embodiment 84: The method of any one of Embodiments 1 to 73, 77, and 78, wherein the cancer is non-small lung cancer and the individual has received one or more prior therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, and wherein the one or more prior therapies comprise an EGFR inhibitor and a bispecific EGF receptor-directed and MET receptor-directed antibody.

Embodiment 85: The method of any one of Embodiments 1 to 73, 77, and 78, wherein the individual has received one or more prior therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, and wherein the one or more prior therapies comprise an EGFR inhibitor and amivantamab.

Embodiment 86: The method of any one of Embodiments 1 to 73, 77, and 78, wherein the cancer is non-small cell lung cancer and the individual has received one or more prior therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, and wherein the one or more prior therapies comprise an EGFR inhibitor and amivantamab.

Embodiment 87: The method of any one of Embodiments 1 to 73 and 77 to 86, wherein the individual has failed one or more prior therapies prior to administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, and wherein the one or more prior therapies is selected from (a) a bispecific EGF receptor-directed and MET receptor-directed antibody, and (b) chemotherapy.

Embodiment 88: The method of Embodiment 87, wherein the one or more prior therapies comprises a bispecific EGF receptor-directed and MET receptor-directed antibody is amivantamab.

Embodiment 89: The method of Embodiment 87, wherein the one or more prior therapies comprises chemotherapy.

Embodiment 90: The method of Embodiment 89, wherein the one or more prior therapies comprising chemotherapy comprises platinum-based chemotherapy.

Embodiment 91: The method of Embodiment 89, wherein the one or more prior therapies comprising chemotherapy does not comprise platinum-based chemotherapy.

Embodiment 92: The method of any one of Embodiments 1 to 91, wherein the individual is administered (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, in an amount that provides a total daily dose of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide selected from (a) more than 10 mg and less than about 45 mg, (b) more than about 45 mg but less than about 200 mg, (c) from about 45 mg to about 60 mg, (d) from about 45 mg to about 90 mg, (e) from about 45 mg to about 120 mg, (f) about 45 mg, (g) about 60 mg, (h) about 75 mg, (i) about 80 mg, (j) about 90 mg, (k) about 100 mg, (l) about 120 mg, (m) about 150 mg, (n) about 175 mg, (o) about 200 mg, and (p) greater than about 120 mg but less than about 200 mg.

Embodiment 93: The method of any one of Embodiments 1 to 92, wherein the individual is administered a malonate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in an amount that provides a total daily dose of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide selected from (a) more than 10 mg and less than about 45 mg, (b) greater than about 45 mg but less than about 200 mg, (c) from about 45 mg to about 60 mg, (d) from about 45 mg to about 90 mg, (e) from about 45 mg to about 120 mg, (f) about 45 mg, (g) about 60 mg, (h) about 75 mg, (i) about 80 mg, (j) about 90 mg, (k) about 100 mg, (l) about 120 mg, (m) about 150 mg, (n) about 175 mg, (o) about 200 mg, and (p) greater than about 120 mg but less than about 200 mg.

Embodiment 94: The method of any one of Embodiments 1 to 93, wherein the individual is administered a malonate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide or the pharmaceutical composition in an amount that is about 30 mg BID, about 40 mg BID, about 45 mg QD, about 50 mg BID, about 60 mg QD, about 75 mg QD, about 90 mg QD, or about 120 mg QD.

Embodiment 95: The method of any one of Embodiments 1 to 94, wherein the individual is administered (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, in an amount that provides a mean unbound concentration of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide in the plasma of the individual that is greater than or equal to 0.1 nM for at least 8 hours following the administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

Embodiment 96: The method of any one of Embodiments 1 to 95, wherein the individual achieves stable disease, a partial response, or a complete response in the individual's target tumors in accordance with RECIST 1.1.

Embodiment 97: The method of Embodiment 96, wherein the response in accordance with RECIST 1.1 is a partial response or a complete response.

Embodiment 98: The method of any one of Embodiments 1 to 97, wherein the individual does not experience any Grade 3 or Grade 4 treatment related adverse events following administration of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof, to the individual.

Embodiment 99: The method of any one of Embodiments 1 to 98, wherein the cancer is a metastatic solid tumor.

### EXAMPLES

The following abbreviations may be used herein:

| | |
|---|---|
| XRPD | X-Ray Powder Diffraction |
| DSC | Differential Scanning Calorimetry |
| DVS | Dynamic vapor sorption |
| NMR | Nuclear magnetic resonance |
| TGA | Thermal Gravimetric Analysis |
| RT | Room Temperature |
| 2-MeTHF | 2-Methyltetrahydrofuran |
| ACN | Acetonitrile |
| CPME | Cyclopentyl methyl ether |
| DCM | Dichloromethane |
| DMSO | Dimethyl sulfoxide |
| EtOAc | Ethyl acetate |
| EtOH | Ethanol |
| IPA | 2-propanol |
| IPAc | Isopropyl acetate |
| MeOH | Methanol |
| MIBK | Methyl isobutyl ketone |
| MTBE | Methyl tert-butyl ether |
| THF | Tetrahydrofuran |

The salts form of Compound I were characterized by various analytical techniques, including XRPD, DSC, TGA, ¹H NMR, and DVS, using the procedures described below.

### XRPD

For XRPD analysis, X'pert³ and Empyrean X-ray powder diffractometers were used. Sample was spread on the middle of a zero-background Si holder. The XRPD parameters used are listed in Table 5 below.

TABLE 5

| Instrument | X′ Pert³ | Empyrean |
|---|---|---|
| Model | Reflection | VT-XRPD |

| | |
|---|---|
| X-Ray | Cu, Kα, Kα1 (Å): 1.540598; |
| | Kα2 (Å): 1.544426 |
| | Kα2/Kα1 intensity ratio: 0.50 |
| X-Ray tube setting | 45 kV, 40 mA |

| | | |
|---|---|---|
| Divergence slit | ⅛° | ⅛° |
| Scan mode | Continuous | Continuous |
| Scan range (°2TH) | 3°~40° | 3°~40° |
| Step size (°2TH) | 0.0263 | 0.0167 |
| Scan step time (s) | 46.67 | 33.02 |
| Test time(s) | 5 min 04 s | 10 min 12 s |

### DSC

DSC and mDSC analyses were performed using a TA Q2000 and Discovery DSC 2500 DSC from TA Instruments. The DSC parameters used are listed in Table 6 below.

TABLE 6

| Parameters | DSC |
|---|---|
| Method | Ramp |
| Sample pan | Aluminum, crimped |
| Temperature | 25° C.~270° C. |
| Heating rate | 10° C./min |
| Purge gas | N₂ |

### TGA

TGA data were collected using a TA Q5000 and Discovery TGA 5500 TGA from TA Instruments. The TGA parameters used are listed in Table 7 below.

TABLE 7

| Parameters | TGA |
|---|---|
| Method | Ramp |
| Sample pan | Aluminum, open |
| Temperature | RT~350° C. |
| Heating rate | 10° C./min |
| Purge gas | N₂ |

### ¹H NMR

¹H solution NMR was collected on Bruker 400M NMR Spectrometer, using DMSO-d₆ or deuterated chloroform as solvent.

### DVS

DVS was measured via a SMS (Surface Measurement Systems) DVS Intrinsic.

The relative humidity at 25° C. was calibrated against deliquescence point of LiCl, Mg(NO₃)₂ and KCl. Parameters for DVS test are listed in Table 8 below.

TABLE 8

| Parameters | DVS | |
|---|---|---|
| Temperature | 25° | C. |
| Sample size | 20~40 | mg |
| Gas and flow rate | N₂, 200 mL/min | |
| dm/dt | 0.002%/min | |
| Min. dm/dt | 10 | min |
| stability duration | | |
| Max. equilibrium time | 180 | min |

| | |
|---|---|
| RH range | Room humidity~95% RH~0% RH~95% RH |
| RH step size | 10% RH (0% RH~90% RH & 90% RH~0% RH) |
| | 5% RH (90% RH~95% RH & 95% RH~90% RH) |

### Example 1. Preparation of a Malonate Salt of Comppund I (Method 1)

A malonate salt of Compound I was prepared as set forth below in Scheme 2. (R)-2-(6-chloropyrimidin-4-yl)-3-(3,5-difluorophenyl)isoxazolidine (Compound A) may be prepared according to methods known to those having ordinary skill in the art, and methods analogous to those described in United States Patent Application Publication No. 2022/0162203A1, the contents of which are incorporated by reference herein for that purpose. 1-cyclopropyl-4-(piperidin-4-yl)piperazine may be prepared using methods known to those having ordinary skill in the art, and methods analogous to those described in PCT Publication No. WO2004087700 A1, the contents of which are hereby incorporated by reference for that purpose.

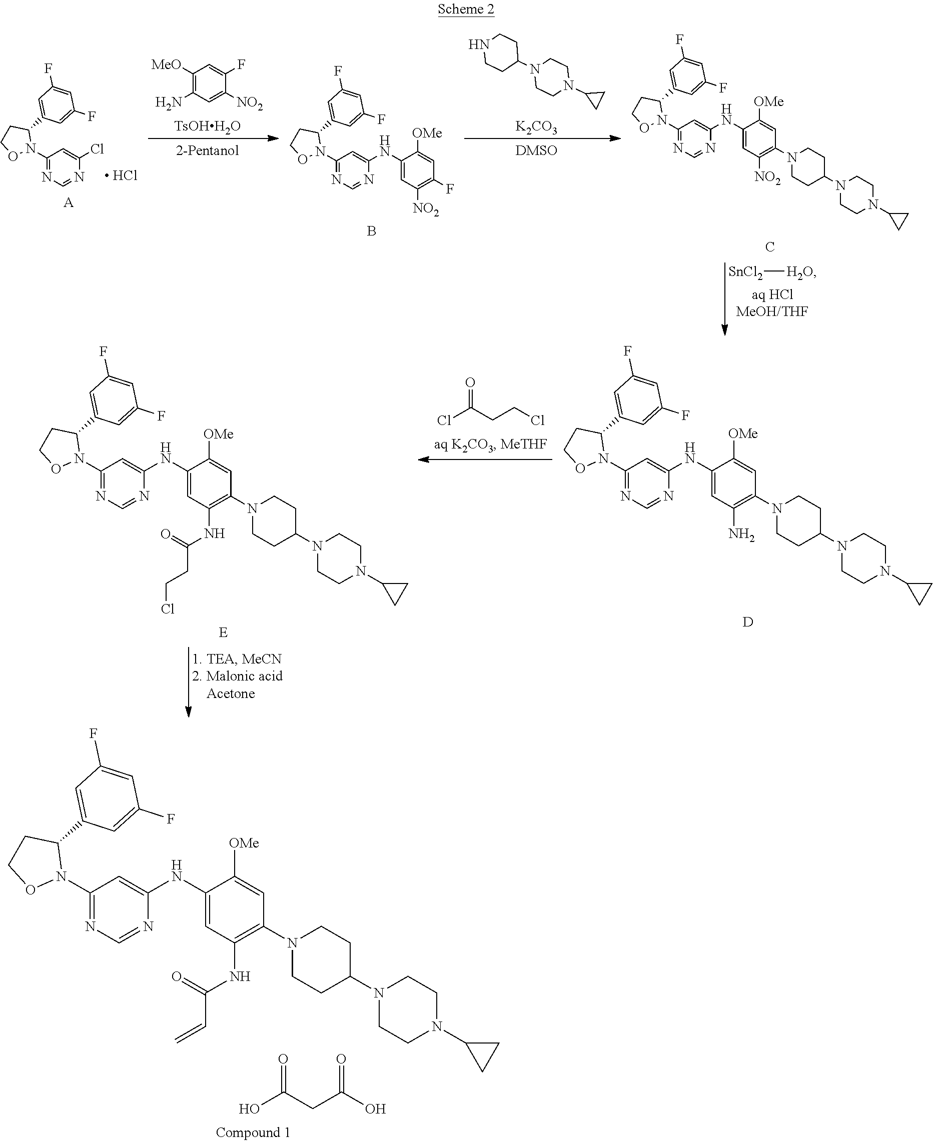

Step 1: A solution of (R)-2-(6-chloropyrimidin-4-yl)-3-(3,5-difluorophenyl)isoxazolidine (Compound A) and 4-fluoro-2-methoxy-5-nitroaniline in 2-pentanol was treated with p-toluenesulfonic acid (TsOH) and the mixture was heated until the reaction was deemed complete. The reaction mixture was subjected to an aqueous workup into a methyl tert-butylether (MTBE)/dichloromethane (DCM) mixture, crystallized from MTBE/heptane, and then dried to give (R)-6-(3-(3,5-difluorophenyl)isoxazolidin-2-yl)-N-(4-fluoro-2-methoxy-5-nitrophenyl)pyrimidin-4-amine (Compound B).

Step 2: A solution of (R)-6-(3-(3,5-difluorophenyl)isoxazolidin-2-yl)-N-(4-fluoro-2-methoxy-5-nitrophenyl)pyrimidin-4-amine (Compound B) from Step 1 and 1-cyclopropyl-4-(piperidin-4-yl)piperazine (3 HCl) in dimethylsulfoxide (DMSO) was treated with potassium carbonate (K₂CO₃) and the mixture was heated until the reaction was deemed complete. The resulting crude product was isolated upon addition of water followed by filtration of the resulting solids, which were recrystallized from 2-methyltetrahydrofuran (2-MeTHF)/heptane and dried to give (R)—N-(4-(4-(4-cyclopropylpiperazin-1-yl)piperidin-1-yl)-2-methoxy-5-nitrophenyl)-6-(3-(3,5-difluorophenyl)isoxazolidin-2-yl)pyrimidin-4-amine (Compound C) as a solid.

Step 3: A solution of R)—N-(4-(4-(4-cyclopropylpiperazin-1-yl)piperidin-1-yl)-2-methoxy-5-nitrophenyl)-6-(3-(3,5-difluorophenyl)isoxazolidin-2-yl)pyrimidin-4-amine (Compound C) from Step 2 was dissolved in a mixture of methanol (MeOH) and tetrahydrofuran (THF), and the resulting solution was treated with tin chloride dihydrate (SnCl2-2H2O) and aqueous hydrochloric acid (aqueous HCl). The resulting mixture was stirred until the reaction was deemed to be complete. The reaction mixture was neutralized and subjected to an aqueous workup into dichloromethane (DCM). (R)-4-(4-(4-cyclopropylpiperazin-1-yl)piperidin-1-yl)-N1-(6-(3-(3,5-difluorophenyl)isoxazolidin-2-yl)pyrimidin-4-yl)-6-methoxybenzene-1,3-diamine (Compound D) was isolated via crystallization from ethyl acetate (EtOAc)/heptane followed by drying.

Step 4: A solution of (R)-4-(4-(4-cyclopropylpiperazin-1-yl)piperidin-1-yl)-N1-(6-(3-(3,5-difluorophenyl)isoxazolidin-2-yl)pyrimidin-4-yl)-6-methoxybenzene-1,3-diamine (Compound D) from Step 3 was dissolved in 2-MeTHF and the resulting solution was treated with K2CO3 and 3-chloropropanoyl chloride. The resulting mixture was stirred until the reaction was deemed to be complete. The reaction mixture was then subjected to an aqueous workup, the solvent was exchanged the acetonitrile (MeCN) and the resulting solution of (R)-3-chloro-N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)propenamide (Compound E) was used directly in the next step without further purification.

Step 5: A solution (R)-3-chloro-N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)propenamide (Compound E) from Step 4 in MeCN was treated with triethylamine (TEA) and the mixture was stirred until the reaction was deemed to be complete. The resulting reaction mixture was then subjected to an aqueous workup into DCM, the solvent was exchanged to acetone, and the solution of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide was treated with malonic acid in acetone to provide Compound I.

### Example 2. Preparation of a Malonate Salt of Compound I (Method 2)

Freebase Compound I was stirred with an equimolar amount of malonic acid in acetone at RT for 14.5 hrs. The slurry was subjected to vacuum drying at 50° C. for about 6 hrs. The malonate salt of Compound I was analyzed by XRPD, DSC, TGA, ¹H NMR, and DVS. FIG. 1A shows an XRPD pattern of a malonate salt of Compound I. FIG. 1D shows DSC and TGA graphs of a malonate salt of Compound I. As shown in the DSC graph, an endotherm at 147.8° C. was observed. As shown in the TGA graph, a weight loss of about 0.7% up to 130° C. was observed. FIG. 1G show a ¹H NMR spectrum of a malonate salt of Compound I. As shown in FIG. 1G, the molar ratio of acid/Compound I was about 1.1, and the residual solvent acetone/Compound I was about 0.06:1 (0.5 wt. %). FIG. 1J shows a DVS graph of a malonate salt of Compound I. As shown in FIG. 1J, a moisture uptake of 0.19% at 25° C./80% relative humidity (RH) range as determined by DVS was observed.

### Example 3. Preparation of a Malonate Salt of Compound I (Method 3)

Freebase Compound I (499.7 mg) and acetone (5 mL) were placed in a 20-mL glass vial to form the freebase solution. Malonic acid (80.0 mg) was dissolved in acetone (3 mL) to form the acid solution. The malonate salt of Compound I (2.0 mg) as prepared according to Example 1 was added to the freebase solution as seeds. The acid solution was added dropwise into the freebase solution with stirring. The resulting suspension was stirred at RT for about 40 hrs. The suspension was centrifuged and the resulting solids were dried at 50° C. under vacuum for 2 hrs. A malonate salt of Compound I (524.6 mg, 90.4%) was obtained and analyzed by XRPD, DSC, TGA, and ¹H NMR. FIG. 1B shows an XRPD pattern of a malonate salt of Compound I. FIG. 1E shows DSC and TGA graphs of a malonate salt of Compound I. As shown in the DSC graph, an endotherm at 149.7° C. was observed. As shown in the TGA graph, a weight loss of about 0.8% up to 130° C. was observed. FIG. 1H show a ¹H NMR spectrum of a malonate salt of Compound I. As shown in FIG. 1H, the molar ratio of acid/Compound I was about 1.0, and the residual solvent acetone/Compound I was about 0.1 (0.7 wt. %).

### Example 4. Preparation of a Malonate Salt of Compound I (Method 4)

Freebase Compound I (5.00 g) and acetone (50 mL) were placed in a 100-mL reactor and stirred at RT to form the freebase solution. Malonic acid (801.0 mg) was dissolved in acetone (30 mL) to form the acid solution. The malonate salt of Compound I (51.0 mg) as prepared according to Example 2 was added to the freebase solution as seeds. The acid solution was added to the freebase solution in 1 hr with stirring. The resulting suspension was stirred at RT for about 41 hrs. The solids were isolated by vacuum filtration and dried under vacuum at 50° C. for 6 hrs followed by at RT for 14 hrs. A malonate salt of Compound I (5.17 g, 88.5%) was obtained and analyzed by XRPD, DSC, TGA, and ¹H NMR. FIG. 1C shows an XRPD pattern of a malonate salt of Compound I. FIG. 1F shows DSC and TGA graphs of a malonate salt of Compound I. As shown in the DSC graph, an endotherm at 149.2° C. was observed. As shown in the TGA graph, a weight loss of about 0.6% up to 130° C. was observed. FIG. 1I shows a ¹H NMR spectrum of a malonate salt of Compound I. As shown in FIG. 1I, the molar ratio of acid/Compound I was about 1.0, and the residual solvent acetone/Compound I was about 0.1 (0.7 wt. %).

### Example 5. Preparation of a Glycolate Salt of Compound I

Freebase Compound I was stirred with an equimolar amount of glycolic acid in acetone at RT for 2 days, followed by vacuum drying at 50° C. for 2 hrs. The glycolate salt of Compound I was analyzed by XRPD, DSC, TGA, ¹H NMR, and DVS. FIG. 2A shows an XRPD pattern of a glycolate salt of Compound I. FIG. 2B shows DSC and TGA graphs of a glycolate salt of Compound I. As shown in the DSC graph, an endotherm at 163.1° C. was observed. As shown in the TGA graph, a weight loss of about 1.3% up to 120° C. was observed. FIG. 2C show a ¹H NMR spectrum of a glycolate salt of Compound I. As shown in FIG. 2C, the molar ratio of acid/Compound I was 1.1, and the molar ratio of residual solvent acetone/Compound I was 0.3 (2.2 wt. %). FIG. 2D shows a DVS graph of a glycolate salt of Compound I. As shown in FIG. 2D, a moisture uptake of 1.4% at 25° C./80% relative humidity (RH) as determined by DVS was observed.

### Example 6. Solubility Screening of Malonate Salt of Compound I

The solubility of the malonate salt of Compound I as prepared according to Example 2 in various solvents at RT was measured. The malonate salt of Compound I (approximately 2 mg) was added to a 3-mL glass vial. The solvent was then added stepwise (50/50/200/200/500/1000 μL) into the vial until the solids were dissolved visually or a total volume of 2 mL was reached. The results are provided below in Table 9.

TABLE 9

| Solvent | Solubility (mg/mL) | Solvent | Solubility (mg/mL) |
|---|---|---|---|
| MeOH | S > 46.0 | CPME | S < 1.1 |
| EtOH | 1.9 < S < 6.3 | 1,4-Dioxane | 2.1 < S < 7.0 |
| IPA | S < 1.2 | Anisole | S < 0.9 |
| Acetone | 1.1 < S < 2.2 | ACN | 6.0 < S < 18.0 |
| MIBK | S < 1.2 | DCM | 20.0 < S < 40.0 |
| EtOAc | S < 1.0 | CHCl₃ | S > 40.0 |
| IPAc | S < 0.9 | n-Heptane | S < 1.1 |
| MTBE | S < 1.0 | Toluene | S < 1.0 |
| THF | 2.1 < S < 7.0 | DMSO | S > 40.0 |
| 2-MeTHF | 1.1 < S < 2.2 | Water | S < 1.2 |

### Example 7: Biodistribution of Compound I after Administration in Mice

### Study Design

Female non-tumor bearing Fox Chase CB17 SCID mice were orally administered a single dose as specified in Table B1.

TABLE B1
Treatment Groups

| | | | | Dose |
|---|---|---|---|---|
| Group | Formulation | ROA | Dose | volume |
| 1 | 30% Captisol in | Oral | 50 mg/kg | 10 |
| | sterile water | | tucatinib | mL/kg |
| 2 | 0.5% citric acid in | Oral | 3 mg/kg of | |
| | sterile water | | Compound I | |

ROA = Route of administration

Brain tissues were excised and plasma was collected from all the animals receiving Compound I, or tucatinib at 1 hour, 4 hours, and 8 hours post-administration. Tucatinib was used as a reference agent. Tucatinib is a clinically approved therapy for HER2 positive breast cancer patients with brain metastases. The harvested brains were rinsed in ice-cold phosphate-buffered saline and weighed. All plasma and brain samples were stored at approximately −80° C. until thawed for LC-MS/MS analysis.

Prior to analysis, brain tissues were homogenized 1:5 with phosphate buffer. Average free brain-to-free plasma ratio (K_{p,uu}) was calculated from individual mice by using total concentration values determined in the brain and plasma at individual time points, or by calculating the area under the curve (AUC) and converting the values to free concentration (or AUC) with in vitro binding parameters determined in the brain (f_{u,b}) and plasma (f_{u,p}) as follows:

Kp,uu=Brain Concentration (or AUC)*fu,b/Plasma Concentration (or AUC)*fu,p.

Free fraction in the brain (f_{u,b}) and in plasma (f_{u,p}) was determined with equilibrium dialysis as follows: The brain tissue homogenate or plasma from the mice were spiked to yield a final concentration of 1 μM Compound I, or 5 μM tucatinib. Compound I and tucatinib were incubated in equilibrium dialysis plate on a shaking incubator at 37° C. with 5% CO₂ at 450 rpm for 8 hours and 24 hours, respectively. Following the incubation, aliquots of the matrix and the buffer samples were sampled and the concentration of Compound I, or tucatinib were determined.

Free fraction in plasma was determined as follows:

$${f}_{u , p} = \frac{Buffer concentration}{Plasma concentration}$$

Free fraction in brain was determined as follows:

$${f}_{u , b} = \frac{1 / ( Dilution Factor )}{( \frac{1}{{f}_{u , b , d}} - 1 ) + 1 / ( Dilution Factor )}$$

where f_{u,b,d} is fraction unbound determined with 20% brain homogenate.

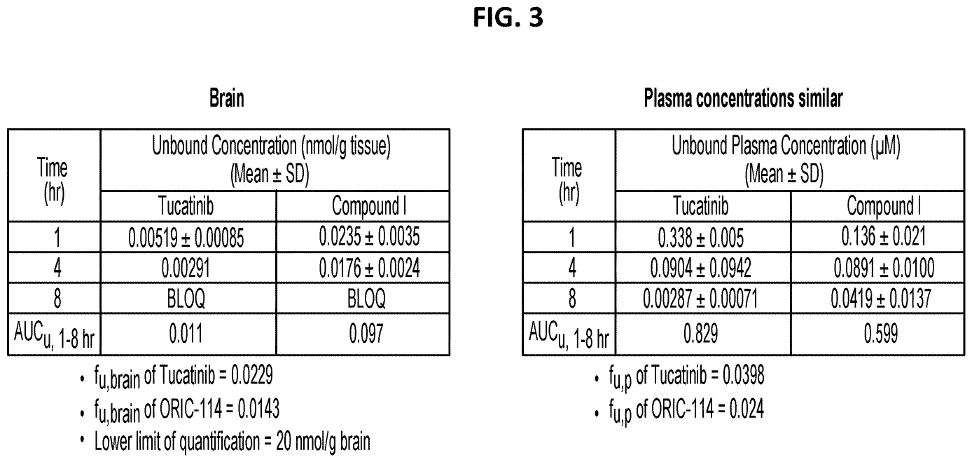

As indicated in FIG. 3, concentrations of unbound tucatinib and Compound I in the brain tissue or plasma were observed over time. Surprisingly, Compound I at a dosage of only 3 mg/kg exhibited similar unbound plasma concentration 4 hours after administration as compared to tucatinib at a dosage of 50 mg/kg. At 8 hours after administration, Compound I at 3 mg/kg achieved an unbound plasma concentration that is about 15-fold of the unbound plasma concentration achieved by tucatinib at 50 mg/kg. See FIG. 3, the table at the right.

More strikingly, Compound I at the 3 mg/kg dosage exhibited a much higher unbound brain concentration at both one-hour and four-hour time points after administration as compared to tucatinib at 50 mg/kg. Both compounds were below the limit of quantitation (BLOQ) after 8 hours. The total area under the curve (AUC) of Compound I at 3 mg/kg during the first eight hours is about 800% higher than that of tucatinib at 50 mg/kg.

The above results demonstrate the excellent bioavailability profile of Compound I in both plasma and brain. Importantly, the superior ability of Compound I to penetrate the blood-brain barrier as compared to tucatinib is striking.

### Example 8: Sensitivity of Compound I on EGFR Mutant Proteins

Compound I was dissolved in DMSO to generate a 10 mM stock solution. Dosing solutions for biochemical assays were prepared by serial dilution in DMSO followed by dilution in assay buffers as described in the experimental procedures. Stock formulations were stored at −80° C. and dosing solutions were prepared for each experiment immediately prior to use.

Activity of human recombinant EGFR wildtype and mutant proteins was measured by the phosphorylation of a peptide substate via chelation-enhanced fluorescence using the sulfonamido-oxine (Sox) chromophore detection format. Compound I was solubilized in DMSO. 16-point half log titrations in 100% DMSO were carried out by a Bravo liquid handler (Agilent) in a 384-well polypropylene plate (Greiner Cat. #781201). Using the Bravo liquid handler, intermediate 1:10 dilutions in a 384-well polypropylene plate were created in assay buffer. For the enzymatic reactions, 5 μL from the intermediate compound dilution plate was transferred to a low volume 384 well, black flat-bottom polystyrene microplate (Greiner Cat. #784076). A separate plate was generated for each enzyme. Next, 5 μL of a 4× ATP/AQT0794 working solution was added to the assay plate. Finally, 10 μL of 2× enzyme was added to start the reactions. The plate was sealed with a PerkinElmer TopSeal-A Plus plate seal (Cat. #6050185) and read at excitation (360 nm) and emission (492 nm) in continuous kinetic mode every 2 minutes for 3 hours at room temperature. Reactions were performed in 50 mM HEPES, pH 7.5, 1 mM ATP, 1.0 mM DTT, 0.015% Brij-35, 0.01% BSA, 1.0 mM EGTA, 10 mM MgCl2, 10 μM EGFR peptide substrate probe from AssayQuant (Cat. #AQT0794) plus recombinant EGFR protein (Table 1). The final DMSO concentration was 2.5%. The various EGFR proteins tested are set forth in Table 10.

TABLE 10

| | | Amount |
|---|---|---|
| | | Added to |
| EGFR Protein | Source | Assay |
| EGFR WT | ThermoFisher Cat. #PV3872 | 2.0 nM |
| EGFR_G719C | SignalChem Cat. #E10-12FG | 5.0 nM |
| EGFR_G719S | SignalChem Cat. #E10-12HG | 1.0 nM |
| EGFR_L718Q | SignalChem Cat. #E10-122G2G | 2.0 nM |
| EGFR_L747S | SignalChem Cat. #E10-122Y2G | 0.5 nM |
| EGFR_L792H | SignalChem Cat. #E10-122J2G | 1.0 nM |
| EGFR d746-750 G724S | SignalChem Cat. #E10-122D3G | 2.0 nM |
| EGFR_d746-750 S768I | SignalChem Cat. #E10-122B3G | 2.0 nM |

Real-time continuous kinetic data were loaded into Dotmatics software where slopes were calculated, converted to % inhibitions and IC₅₀s were generated using the four-parameter non-linear regression curve fit. Reactions lacking enzymes were used as 100% inhibition controls. After addition of recombinant EGFR and its substrates ATP and AQT0794, the activity of EGFR was measured by quantification of the phosphorylation of the AQT0794 peptide substate via chelation-enhanced fluorescence using the sulfonamido-oxine (Sox) chromophore detection format with diminishing signal indicative of inhibition of EGFR activity. The IC₅₀s of Compound I against the various EGFR mutant proteins are set forth in Table 11.

TABLE 11

| EGFR Protein | Compound I IC50 [nM] |
|---|---|
| EGFR WT | 2.3 ± 1.1 |
| EGFR_G719C | 0.6 |
| EGFR_G719S | 0.9 |
| EGFR_L718Q | 3.7 |
| EGFR_L747S | 1.5 |
| EGFR_L792H | 16 |
| EGFR_d746-750 G724S | 3.5 |
| EGFR_d746-750 S768I | 1.0 |

### Example 9: Sensitivity of Compound I on Cells Comprising EGFR Mutant Proteins

A cell-based assay was used to evaluate the potency of Compound I on cells where the growth of the cells is either driven by EGFR wild type (WT) or cells comprising atypical mutant EGFR proteins. Cell viability was assessed following treatment with compounds and the assay developed with CellTiterGlo (CTG) reagent that measures ATP levels in cells. Compound I was dissolved in DMSO to generate a 10 mM stock solution. Dosing solutions were prepared by serial dilution in DMSO followed by dilution in complete media to result in a 0.4% DMSO in the assay. Ba/F3 cells expressing EGFR mutants EGFR_G719S, EGFR_G724S, EGFR_L718Q, EGFR_L718V, EGFR_L858R/1L718Q, EGFR_L858R/1L718V, EGFR_del19/L792K. EGFR_del19/G796S were generated following co-transfection of the EGFR mutant of interest with PhiC31 integrase. Two days following transfection, the media was changed and replaced with media lacking mIL3 with the addition of human EGF (hEGF) as well as puromycin to select stable cell lines. After the cell lines were established genomic DNA (gDNA) was extracted to confirm the sequence of the construct of interest. The culture Media for Ba/F3 EGFR mutants was RPMI+10% FBS+2 mM L-Glut±10 ng/ml hEGF+0.5 pig/ml puromycin. The following reagents were used to conduct the assays: RPMI1640 with phenol red (Corning, Cat #: 15-040-CV); Fetal Bovine Serum (FBS) (Omega Scientific, Cat #: FR-11); Human Epidermal Growth Factor (hEGF) (Cell Signaling Cat #8916SC); L-Glutamine (Corning, Cat #: 25-005-CI); Puromycin (Gibco, Cat #A11138-03); assay media was the same as culture medium; CellTiter-Glo® 2.0 Cell Viability Assay (Promega, Cat #G9243), DMSO (Sigma, Cat #: D2660). The following equipment was used to conduct the assays: Bravo Liquid Handler (Agilent #G5523BA); and the following supplies: 96-well Non-Sterile Polypropylene V Bottom Plates (Corning, Cat #: 3363); 384-well Sterile White with Flat Clear Bottom Plates (Greiner, 781098); and PerkinElmer TopSeal-A Plus plate seal (Cat. #6050185).

Ba/F3 cell lines expressing either EGFR WT or mutant proteins were cultured in complete culture media at exponential growth phase for at least one week prior to assay. The different cell suspensions were counted and seeded into 384-well Sterile White Flat Clear Bottom Plates using a Bravo liquid handler, according to the appropriate cell number in 36 μl of media per well as follows: (a) Ba/F3-WT-EGFR: 2000 cells per well of a 384 well plate; (b) Ba/F3-G719S-EGFR: 2000 cells per well of a 384 well plate; (c) Ba/F3 G724S-EGFR: 1000 cells per well of a 384 well plate; (d) Ba/F3-L718Q-EGFR: 1000 cells per well of a 384 well plate; (e) Ba/F3-L718V-EGFR: 1000 cells per well of a 384 well plate; (f) Ba/F3-L858R/L718Q-EGFR: 1000 cells per well of a 384 well plate; (g) Ba/F3-L858R/L718V-EGFR: 1000 cells per well of a 384 well plate; (h) Ba/F3-del19/L792H-EGFR: 1000 cells per well of a 384 well plate; (i) Ba/F3-del19/G796S-EGFR: 1000 cells per well of a 384 well plate.

Compound I was solubilized in DMSO as 10 mM stock solutions. These were stored as aliquots at −80° C. and used only once after thawing. The highest concentration of Compound I in the final assay plate was 500 nM. To prepare the compound dilutions intermediate dilution plates were prepared using the Bravo liquid handler by performing 12-point ⅓-fold serial dilutions in DMSO to a final concentration of 250-fold the concentration in the final assay plate, followed by a 1/25 dilution into complete assay media. Compound dilutions in media were added to cells (final assay plate) by dispensing 4 μL into cells plated the same day in 36 μl of medium of media. Plates were placed in a 37° C., 5% CO2 incubator. Following 72 hours of incubation, plates were left to equilibrate at room temperature before the addition of 1:1 ratio of CTG 2.0 using the Bravo liquid handler. The plates were sealed with a PerkinElmer TopSeal-A Plus plate seal and incubated at room temperature for 10 minutes before reading the luminescence on a plate reader (Tecan—SPARK). The luminescence is a direct readout of the presence of ATP in the cells. Data from a Tecan SPARK plate reader were transferred to an Excel file and entered into the Dotmatics database. Curves were fit with a four-parameter model and absolute EC50s calculated by Dotmatics software. DMSO wells were averaged to define the high control (100% growth). The average of the highest concentration of the positive control compound (afatinib) was used to define 0% growth and used to lock the bottom of the curves. The tops of the curves were not fixed. The Absolute EC₅₀s were calculated as the compound concentration that gave 50% growth. The average absolute EC₅₀ and ±standard deviations of Compound I determined in each cell line are set forth in Table 12.

TABLE 12

| Ba/F3 EGFR cell lines | Compound I EC₅₀ [nM] |
|---|---|
| WT | 6.6 ± 4.3 |
| EGFR_G719S | 0.7 ± 0.1 |
| EGFR_G724S | 1.4 ± 0.9 |
| EGFR_L718Q | 27.1 ± 6.4 |
| EGFR_L718V | 0.9 ± 0.8 |
| EGFR_L858R/L718Q | 0.8 ± 0.5 |
| EGFR_L858R/L718V | 0.2 ± 0.2 |
| EGFR_del19/L792H | 2.5 ± 1.8 |
| EGFR_del19/G796S | 1.1 ± 0.7 |

### Example 10: Clinical Evaluation of Compound I in Human Subjects Having Non-Small Cell Lung Cancer Comprising EGFR Exon 20 Insertion Mutations, HER2 Exon 20 Insertion Mutations, or HER2 Amplification or Overexpression

A clinical study was conducted using Compound I in human subjects having non-small cell lung cancer (NSCLC) comprising EGFR exon 20 insertion mutations, HER2 exon 20 insertion mutations, or HER2 amplification or overexpression. A total of 50 human subjects having non-small cell lung cancer comprising one or more EGFR exon 20 insertion mutations, HER2 exon 20 insertion mutations, or HER2 amplification or overexpression were enrolled in the study as of Jul. 31, 2023 with data collected as of Sep. 26, 2023. The subjects were enrolled into one of ten dosing cohorts that were each orally administered Compound I in the form of a hemifumarate salt as shown in Table 13 (QD=once per day; BID=twice per day).

TABLE 13

| Cohort | No. of | Dose of Compound I in the |
|---|---|---|
| No. | subjects | form of a hemifumarate salt |
| 1 to 5 | 18 | Less than 45 mg total daily |
| | | dose (6 mg QD, 12 mg QD, |
| | | 20 mg QD, 30 mg QD, and |
| | | 20 mg BID) |
| 6 | 7 | 45 mg QD |
| 7 | 9 | 60 mg QD |
| 8 | 6 | 75 mg QD |
| 9 | 3 | 90 mg QD |
| 10 | 7 | 30 mg BID |

The general characteristics of the enrolled subjects are described in Table 14. Of the subjects enrolled having NSCLC with EGFR exon 20 insertion mutations: (a) 81% were treated with one or more prior EGFR exon 20 targeted agents; (b) 19% received multiple EGFR exon 20 targeted agents; and (c) 86% presented with CNS metastases at baseline. Of the subjects enrolled having NSCLC with HER2 exon 20 insertion mutations: (a) 30 were treated with a prior HER2 targeted agent; and (b) 38% were treated with CNS metastases at baseline (n=number; N/A=not applicable)

TABLE 14

| | EGFR exon 20 | | HER2 exon 20 | | HER2-positive | | Total | |
|---|---|---|---|---|---|---|---|---|
| | (n = 21) | | (n = 24) | | (n = 5) | | (n = 50) | |
| Age, years, | 63 | (31, 80) | 63 | (25, 86) | 63 | (25, 86) | 63 | (25, 86) |
| median (range) | | | | | | | | |
| Females, n (%) | 10 | (48) | 11 | (46) | 3 | (60) | 24 | (48) |
| ECOG | | | | | | | | |
| performance | | | | | | | | |
| score, n (%) | | | | | | | | |
| 0 | 1 | (5) | 10 | (42) | 3 | (60) | 14 | (28) |
| 1 | 20 | (95) | 14 | (58) | 2 | (40) | 36 | (72) |
| Non-smoker, n | 12 | (57) | 16 | (68) | 3 | (60) | 31 | (62) |
| (%) | | | | | | | | |
| Prior lines of | 2 | (1, 6) | 2 | (0, 7) | 4 | (1, 7) | 2 | (0, 7) |
| therapies, | | | | | | | | |
| median (min, | | | | | | | | |
| max) | | | | | | | | |
| Prior therapies, | | | | | | | | |
| n (%) | | | | | | | | |
| Chemotherapy | 21 | (100) | 23 | (96) | 5 | (100) | 49 | (98) |
| EGFR targeted | 18 | (86) | 1 | (4) | N/A | | 19 | (38) |
| agents | | | | | | | | |
| EGFR exon 20 | 17 | (81) | N/A | | N/A | | 17 | (34) |
| targeted agents | | | | | | | | |
| Amivantamab | 15 | (71) | N/A | | N/A | | 15 | (30) |
| Mobocertinib | 4 | (19) | N/A | | N/A | | 4 | (8) |
| Other (CLN- | 2 | (10) | N/A | | N/A | | 2 | (4) |
| 081, BLU-451) | | | | | | | | |
| HER2 targeted | N/A | | 7 | (30) | 3 | (60) | 10 | (20) |
| agents | | | | | | | | |
| CNS metastases | 18 | (86) | 9 | (38) | 1 | (20) | 28 | (56) |
| at baseline, n | | | | | | | | |
| (%) | | | | | | | | |

The oral administration of Compound I in the form of a hemifumarate to the subjects described above was associated with Grade 1 and 2 treatment-related adverse events (TRAEs) at each dose level (assigned as per NCI Common Terminology Criteria for Adverse Events v5.0). There were infrequent dose reductions and discontinuations. The TRAEs that occurred in equal to or greater than 10% of subjects in the study are set forth in Tables 15 to 18 (TDD=total daily dose; n=number; N/A=not applicable; the term rash includes the following terms: acne, dermatitis, dermatitis acneiform, eczema, hand dermatitis, and rash).

TABLE 15
<45 mg TDD
(n = 18)

| Preferred | Grade 1, | | Grade 2, | | Grade 3, | ≥Grade 4, |
|---|---|---|---|---|---|---|
| Term | n (%) | | n (%) | | n (%) | n (%) |
| Rash | 6 | (33) | 4 | (22) | N/A | N/A |
| Diarrhea | 2 | (11) | 2 | (11) | | |
| Stomatitis | 4 | (22) | 2 | (11) | N/A | N/A |
| Paronychia | 1 | (6) | 2 | (11) | N/A | N/A |
| Pruritis | 2 | (11) | N/A | | N/A | N/A |
| Nausea | 1 | (6) | N/A | | N/A | N/A |
| Decreased | N/A | | 1 | (6) | N/A | N/A |
| appetite | | | | | | |

| | | | | | |
|---|---|---|---|---|---|
| Vomiting | 2 | (11) | N/A | N/A | N/A |

Dose reductions = 2 (18)
Dose discontinuations = 1 (9)

TABLE 16
45 mg-60 mg TDD
(n = 23)

| Preferred | Grade 1, | | Grade 2, | | Grade 3, | ≥Grade 4, |
|---|---|---|---|---|---|---|
| Term | n (%) | | n (%) | | n (%) | n (%) |
| Rash | 6 | (26) | 6 | (26) | N/A | N/A |
| Diarrhea | 7 | (30) | 2 | (9) | 2 (9) | N/A |
| Stomatitis | 2 | (9) | 2 | (9) | 1 (4) | N/A |
| Paronychia | 4 | (17) | 4 | (17) | N/A | N/A |
| Pruritis | 4 | (17) | 2 | (9) | 1 (4) | N/A |
| Nausea | 2 | (9) | 2 | (9) | N/A | N/A |
| Decreased | 5 | (22) | 1 | (4) | N/A | N/A |
| appetite | | | | | | |

| | | | | | |
|---|---|---|---|---|---|
| Vomiting | 2 | (9) | N/A | N/A | N/A |

Dose reductions = 3 (13)
Dose discontinuations = 1 (4)

TABLE 17
≥75 mg TDD
(n = 9)

| Preferred | Grade 1, | Grade 2, | Grade 3, | ≥Grade 4, |
|---|---|---|---|---|
| Term | n (%) | n (%) | n (%) | n (%) |
| Rash | 4 (44) | 1 (11) | N/A | N/A |
| Diarrhea | 2 (22) | 2 (22) | 1 (11) | N/A |
| Stomatitis | 2 (22) | 2 (22) | N/A | N/A |
| Paronychia | 2 (22) | 1 (11) | N/A | N/A |
| Pruritis | 1 (11) | 1 (11) | N/A | N/A |
| Nausea | 1 (11) | 1 (11) | 1 (11) | N/A |
| Decreased | N/A | N/A | N/A | N/A |
| appetite | | | | |
| Vomiting | 1 (11) | 1 (11) | 1 (11) | N/A |

Dose reductions = 3 (33)
Dose discontinuations = N/A

TABLE 18

| Preferred Term | All Grades, n (%) | |
|---|---|---|
| Rash | 27 | (54) |
| Diarrhea | 20 | (40) |
| Stomatitis | 15 | (30) |
| Paronychia | 14 | (28) |
| Pruritis | 11 | (22) |
| Nausea | 8 | (16) |
| Decreased appetite | 7 | (14) |
| Vomiting | 7 | (14) |
| Dose reductions | 8 | (16) |
| Dose discontinuations | 2 | (4) |

Duration of treatment and antitumor responses achieved in evaluable subjects with EGFR exon 20 insertion mutated NSCLC (dose level greater than or equal to 45 mg TDD and at least one post-baseline tumor assessment) who were administered Compound I in the form of a hemifumarate salt are set forth in Tables 19 and 20, respectively.

TABLE 19

| Subject | | | | Dosing | Duration of |
|---|---|---|---|---|---|
| no. | Age | Sex | Race | cohort | treatment (days) |
| 1 | 51 | Male | Asian | 60 QD | 20 |
| 2 | 66 | Female | White | 90 QD | 28 |
| 3 | 36 | Female | Not Reported | 75 QD | ongoing |
| 4 | 57 | Female | Asian | 45 QD | 28 |
| 5 | 60 | Female | Asian | 90 QD | 52 |
| 6 | 63 | Male | Asian | 30 BID | ongoing |
| 7 | 78 | Female | Asian | 45 QD | 84 |
| 8 | 49 | Male | Asian | 60 QD | 33 |
| 9 | 74 | Female | Not Reported | 75 QD | 90 |
| 10 | 77 | Male | Asian | 30 BID | ongoing |
| 11 | 63 | Male | Asian | 60 QD | ongoing |
| 12 | 55 | Female | Asian | 75 QD | 109 |
| 13 | 80 | Male | Asian | 45 QD | ongoing |
| 14 | 64 | Male | Asian | 45 QD | 134 |
| 15 | 56 | Female | Asian | 75 QD | 20 |

TABLE 20

| Subject | CNS | Prior EGFR | Best percent change in the sum of |
|---|---|---|---|
| no. | disease | therapy | target lesions from baseline (%) |
| 1 | Yes | Yes | 43 |
| 2 | Yes | Yes | 20 |
| 3 | Yes | No | 10 |
| 4 | Yes | No | 0 |
| 5 | Yes | Yes | 0 |
| 6 | Yes | Yes | 0 |
| 7 | No | Yes | −2 |
| 8 | Yes | Yes | −6 |
| 9 | No | Yes | −6 |
| 10 | Yes | Yes | −12 |
| 11 | Yes | No | −28 |
| 12 | Yes | Yes | −33 |
| 13 | Yes | No | −35 |
| 14 | Yes | Yes | −43 |
| 15 | Yes | Yes | −100 |

Duration of treatment and antitumor responses achieved in evaluable subjects with HER2 exon 20 insertion mutate NSCLC (dose level greater than or equal to 45 mg TDD and at least one post-baseline tumor assessment) who were administered Compound I in the form of a hemifumarate salt are set forth in Tables 21 and 22, respectively.

TABLE 21

| Subject | | | | Dosing | Duration of |
|---|---|---|---|---|---|
| no. | Age | Sex | Race | cohort | treatment (days) |
| 1 | 63 | Male | Asian | 60 QD | 28 |
| 2 | 60 | Male | Asian | 30 BID | 54 |
| 3 | 62 | Male | Asian | 60 QD | 2 |
| 4 | 58 | Male | Asian | 60 QD | 28 |
| 5 | 71 | Female | Asian | 45 QD | Ongoing |
| 6 | 58 | Female | Asian | 75 QD | 179 |
| 7 | 73 | Female | Asian | 30 BID | 79 |
| 8 | 59 | Male | Asian | 30 BID | Ongoing |
| 9 | 78 | Female | Asian | 75 QD | 47 |
| 10 | 66 | Female | Asian | 45 QD | Ongoing |
| 11 | 66 | Female | Asian | 30 BID | Ongoing |
| 12 | 63 | Female | Asian | 90 QD | Ongoing |
| 13 | 67 | Male | White | 30 BID | Ongoing |

TABLE 22

| Subject | CNS | Prior HER2 | Best percent change in the sum of |
|---|---|---|---|
| no. | disease | therapy | target lesions from baseline (%) |
| 1 | Yes | No | 31 |
| 2 | No | Yes | 11 |
| 3 | No | No | 9 |
| 4 | Yes | No | 5 |
| 5 | Not Done | No | 0 |
| 6 | No | No | 0 |
| 7 | No | Yes | −3 |
| 8 | No | No | −19 |
| 9 | No | No | −31 |
| 10 | Yes | No | −33 |
| 11 | Yes | No | −34 |
| 12 | No | Yes | −55 |
| 13 | No | No | −100 |

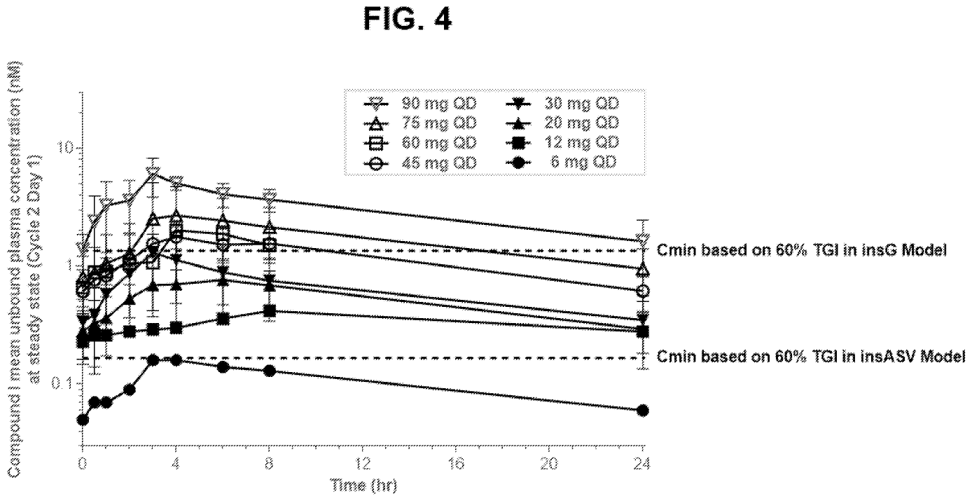

FIG. 4 shows the mean unbound plasma concentration of Compound I at steady state from dosing cycle 2, day 1 in human subjects administered Compound I as described in Example 10.

### Example 11: Clinical Evaluation of Compound I in a Human Subject with EGFR Exon 20 Mutated NSCLC and Active CNS Metastases that Progressed on Prior EGFR Exon 20 Therapy

A 55-year old female subject having EGFR exon 20 mutated non-small cell lung cancer was enrolled in a clinical study relating to Compound I. The cancer in the subject had been previously treated with chemotherapy (pemetrexed/cisplatin) and amivantamab. The subject presented with four active CNS non-target lesions that were previously untreated, and for which the subject had not undergone prior surgery or radiation treatment. The subject was enrolled in a dosing cohort that received 75 mg QD of Compound I in the form of a hemifumarate salt. Following dosing cycle 1 (1 cycle=28 days), the subject exhibited a systemic anti-tumor response (60% reduction of all target and non-target lesions), followed by complete response at the end of dosing cycle 2 (100% reduction of all target and non-target lesions) that was subsequently confirmed. Following dosing cycle 1, the subject exhibited a 100% reduction of all 4 CNS lesions, which was confirmed after completion of dosing cycle 2. During the reporting period (data cutoff of Sep. 26, 2023), the only EGFR-related adverse events were Grade 2 mucositis and Grade 2 paronychia, assessed as related to Compound I. The subject was on dosing cycle 9 and was continuing dosing Compound I at the time of data cutoff.

### Example 12: Clinical Evaluation of Compound I in a Human Subject with HER2 Exon 20 Mutated NSCLC

A 67-year old, male subject having HER2 exon 20 mutated NSCLC was enrolled in a clinical study relating to Compound I. The cancer in the subject had been previously treated with pemetrexed/cisplatin, ipilimumab/nivolumab/carboplatin, and pemetrexed/carboplatin/pembrolizumab. The subject was enrolled in a dosing cohort that received 30 mg BID of Compound I in the form of a hemifumarate salt. Following dosing cycle 1 (1 cycle=28 days dosing at 30 mg BID), the subject exhibited a partial response (100% reduction of all target lesions; non-targeted lesions non-CR/non-PD), which was confirmed at the end of dosing cycle 2 (100% reduction of all target lesions). During the reporting period (data cutoff of Sep. 26, 2023), the only EGFR-related adverse events were Grade 2 rash and Grade 2 nausea, assessed as related to Compound I. The subject was on dosing cycle 3 and was continuing dosing Compound I at the time of data cutoff.

### Example 13: A Phase 1. Open-Label. Single Dose Study to Evaluate the Relative Bioavailability of Two Formulations (Capsule and Tablet) of Compound I in Healthy Subjects

A Phase 1, open-label, single dose study was conducted in healthy human subjects to evaluate the relative bioavailability of two formulations of Compound I. The first formulation of Compound I (Treatment A) was in the form of a capsule comprising about 15 mg of Compound I in the form of a hemifumarate, while the second formulation of Compound I (Treatment B) was in the form of a tablet comprising about 10 mg of Compound I in the form of a malonate and pharmaceutically acceptable excipients. The primary objective of the study was to evaluate the relative bioavailability (BA) of Treatment A and Treatment B administered as a single oral dose under fasting conditions in healthy adult subjects. A secondary objective was to evaluate the safety and tolerability of single dose administration of Compound I Treatment B under fasting conditions. The endpoints of the study were AUC0-last, AUC0-24, AUC0-inf, AUC % extrap, Cmax, Tmax, Kel, t½, CL/F, and Vz/F for Treatment A and Treatment B. The endpoints also included Aetl-t2, Ae, CLr, Fet1-t2, and Fe from Treatment B in period 2. A secondary endpoint included treatment-emergent AEs (TEAEs), clinical laboratory tests, vital signs, and 12 lead electrocardiogram (ECGs).

Methodology: The study was conducted as an open-label, single-dose study. Screening of subjects occurred within 28 days prior to the first dosing. The study was conducted as a randomized, 2-period, crossover study to assess the relative BA of Treatment A and Treatment B. On Day 1 of Period 1 and of Period 2, a single oral dose of Treatment A or Treatment B were administered under fasting conditions. Blood samples for PK were collected pre-dose and through 216 hours post-dose. Urine samples for PK were collected pre-dose and through 48 hours post-dose from subjects randomized to Period 2 Treatment B (tablets). There was a washout period of at least 10 days between dosing in each period. Safety was monitored throughout the study by repeated clinical and laboratory evaluations. Discontinued subjects were not replaced. All 30 enrolled subjects completed the study and were included in the PK and safety analyses.

60 mg Compound I in the form of Treatment B (6×10 mg tablets) was administered at Hour 0 on Day 1 under fasting conditions along with approximately 240 mL of water after a 10-hour fast. The total duration of participation including the screening period for each subject was approximately 8 weeks. There were 2 periods of approximately 14 days each; washout phase was 10 days between both doses. 60 mg of Compound I in the form of Treatment A (4×15 mg capsules) was administered to subjects at Hour 0 on Day 1 under fasting conditions along with approximately 240 mL of water after a 10-hour fast.

Plasma samples for the analysis of Compound I were collected at the following time points relative to dosing on Day 1 of each period: pre-dose and at 0.5, 1, 2, 3, 4, 5, 6, 7, 8, 10, 12, 16, 24, 36, 48, 72, 96, 120, 144, and 216 hours post-dose. Safety was evaluated by TEAEs, clinical laboratory tests, vital signs, 12 lead ECGs, and physical examinations. An analysis of variance (ANOVA) was performed on the natural log (ln)-transformed AUC0-last, AUC0-inf, and Cmax. The ANOVA model included sequence, treatment, and period as fixed effects, and subject nested within sequence as a random effect, and included calculation of least-squares means (LSMs) as well as the difference between treatment LSMs. Ratios of LSMs were calculated using the exponentiation of the difference between treatment LSMs from the analyses on the In-transformed AUC0-last, AUC0-inf, and Cmax. Similar relative bioavailability (BA) were concluded if the 90% confidence intervals (CIs) for the ratios of LSMs of In-transformed AUC0 last, AUC0 inf, and Cmax between the 2 treatments fell within 80.00% and 125.00%. Safety was evaluated by clinical laboratory tests, physical examination, vital signs, 12 lead ECGs, and adverse events (AEs). There was no inferential statistics performed.

PK results: The individual Compound I concentration versus time profiles were well characterized following a single oral dose of 60 mg capsules and 60 mg tablets. Plasma Compound I concentrations were quantifiable in 27 out of 30 subjects following 60 mg capsules and 29 out of 30 subjects following 60 mg tablets at 0.5 hour (i.e., the first post-dose time point), and were quantifiable in 22 out of 30 subjects following 60 mg capsules and 21 out of 30 subjects following 60 mg tablets through 144 hours. Plasma Compound I concentrations were quantifiable in only 4 subjects and 8 subjects at 216 hours following capsules and tablets, respectively. The arithmetic mean concentration versus time profiles following both treatments were nearly identical through the entire sampling interval. Peak mean Compound I values were similar following both treatments and were reached at 6 hours following 60 mg capsules (18.39 ng/mL) and 7 hours following 60 mg tablets (18.87 ng/mL). Plasma Compound I overall (AUCs) and peak (Cmax) exposures were similar following administration of 60 mg capsules and 60 mg tablets. Median plasma Compound I Tmax and Tlag were similar following both treatments. Additionally, arithmetic mean plasma Compound I t½, CL/F, and Vz/F were also similar between both treatments. Subject 13 vomited prior to Tmax but treatment ratios of PK parameters were within the range of all subjects and was included in the analysis. Plasma Compound I overall (AUCs) and peak (Cmax) exposures were similar following administration of 60 mg tablets compared to 60 mg capsules. Additionally, the 90% CIs around the GMRs for all parameters tested were within 80.00% to 125.00% and included the value of 100.00%. The intra-subject CV % was relatively low and ranged from approximately 15.2% to 18.4%.

Safety results: There were no deaths, serious adverse events (SAEs), or TEAEs leading to subject discontinuation in this study. Overall, 14 (47%) subjects reported TEAEs, including 8 (27%) subjects following 60 mg capsules and 7 (23%) subjects following 60 mg tablets. It was determined that 12 (40%) subjects overall experienced TEAEs that were related to the study drug, including all 8 (27%) subjects following 60 mg capsules and 5 (17%) subjects following 60 mg tablets. Of the 14 subjects who reported TEAEs, 10 (33%) subjects experienced events with the maximum severity of mild, including 5 (17%) subjects following 60 mg capsules and 6 (20%) subjects following 60 mg tablets. Four (4 [13%]) subjects overall experienced events with the maximum severity of moderate, including 3 (10%) subjects following 60 mg capsules and 1 (3%) subject following 60 mg tablets. No severe TEAEs were reported. Overall, the most frequently reported TEAEs were constipation, dry skin, papular rash, and epistaxis, each reported by 3 (10%) subjects and considered mild in severity. There were no clinically significant findings or trends noted in the clinical laboratory tests, vital signs, 12-lead ECG assessments, or physical examination relative to subject safety.

Conclusions: Plasma Compound I AUC0-last, AUC0-inf, and Cmax were similar following administration of 60 mg tablets compared to 60 mg capsules since the 90% CIs around the GMRs for all parameters tested were within 80.00% to 125.00%. Plasma Compound I t½ was similar following 60 mg tablets compared to 60 mg capsules, with arithmetic mean values of approximately 29 hours in both treatments. Median Compound I Tmax was reached at the same time following 60 mg tablets and 60 mg capsules (approximately 7 hours post-dose). Following Compound 160 mg tablets, less than 1% of the dose was recovered in urine. A single oral dose of 60 mg Compound I (capsule or tablet) administered under fasting conditions appeared to be generally safe and well tolerated in the health adult subjects in this study.

What is claimed is:

1. A method of treating cancer in an individual in need thereof, wherein in the cancer in the individual has been determined to comprise one or more atypical epidermal growth factor receptor (EGFR) mutations, comprising administering to the individual (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

2. The method of claim 1, wherein the one or more atypical epidermal growth factor receptor (EGFR) mutations in the cancer of the individual comprises one or more (a) P-loop and alpha C-helix compressing (PACC) EGFR mutations, (b) Exon18 mutations, (c) Exon19 mutations, (d) Exon20 point mutations, (e) Exon21 mutations, (f) mutations in the extracellular domain of EGFR, (g) mutations in the transmembrane domain of EGFR, and (h) Exon20 insertion mutations, provided that the Exon20 insertion mutations do not include EGFR_Exon20insNPH, EGFR_Exon20insSVD, EGFR_Exon20insFQEA, EGFR Exon20insH, and EGFR Exon20insASV, EGFR Exon20insYVMA.

3. The method of claim 1, wherein the more atypical epidermal growth factor receptor (EGFR) mutations in the cancer of the individual is selected from the group consisting of EGFR_A702T, EGFR_E709A, EGFR_E709A G719A, EGFR_E709A G719S, EGFR E709K, EGFR E709K G719S, EGFR E709 T710delinsD, EGFR E709 T710del insD S22R, EGFR_L718Q, EGFR L718Q L858R, EGFR_L718V, EGFR_L718V L858R, EGFR_G719A, EGFR_G719A D761Y, EGFR_G719A L861Q, EGFR_G719A R776C, EGFR_G719A S768L, EGFR_G719C, EGFR_G719C S768L, EGFR_G719S, EGFR_G719S L861Q, EGFR_T725M, EGFR_G719S S768L, EGFR_S720P,EGFR_E736K, EGFR_G724S, EGFR_G724S Exl9del, EGFR_G724S L858R, EGFR_I740dupIPVAK, EGFR_E746_A750del A647T, EGFR_E746_A750del G724S, EGFR_E746_A750del S768L, EGFR_E746_A750del R675W, EGFR_E746_T751del insV, EGFR_E746_T751del insV S768C, EGFR T751_I759 delinsN, EGFR L747P, EGFR_L747S, EGFR_L747S V774M, EGFR_L747S L858R, EGFR_L747_S752del A755D, EGFR_A750_I759del insPN, EGFR_S752_I759del V769M, EGFR_K757M L858R, EGFR_K757R, EGFR_D761N, EGFR_A767dupASV, EGFR_S768C, EGFR_S768I, EGFR_S768I V769L, EGFR_S768I V774M, EGFR_S768I L858R, EGFR_S768I L861Q, EGFR_S768dupSVD, EGFR_V769L, EGFR_V769M, EGFR_N771G, EGFR_H773dup, EGFR_V774M, EGFR_R776C, EGFR_R776H, EGFR_G779F, EGFR_L792H, EGFR_Exl9del L792H, EGFR_G796S, EGFR_V774M, EGFR_S784F, EGFR_Exl9del G796S, EGFR_L833V, EGFR_V834L, EGFR_T854L, EGFR_Exl9del T854L, EGFR_L858R L792H, EGFR_L858R C797S, EGFR_L858R T854S, EGFR_L861Q, EGFR_L861R, EGFR_S811F, EGFR_A763insFQEA, EGFR_A763insLQEA, EGFR_E746_A750delL41W, EGFR_E746_A750delR451H, EGFR_K754E, EGFR_L747_E749del, EGFR_A750P, EGFR_L747_T751del, and EGFR_L833F.

4. The method of claim 1, wherein the (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is administered to the individual in the form of a fumarate, hemi-fumarate, glycolate or malonate salt.

5. The method of claim 4, wherein the (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is administered to the individual in the form of a malonate salt.

6. The method of claim 5, wherein the malonate salt is in a crystalline form.

7. The method of claim 6, wherein the crystalline form of the malonate salt maintains at least 95% of the crystalline form following storage in an open container for at least 7 days at 40° C. and 75% relative humidity, and wherein the amount of the crystalline form of the salt is measured by XRPD.

8. The method of claim 7, wherein the crystalline form of the malonate salt exhibits an XRPD pattern comprising a peak at 5.9±0.2 degrees 2-theta.

9. The method of claim 5, wherein the malonate salt of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide is in crystalline form, and wherein the crystalline form exhibits (a) an XRPD pattern comprising a peak at 5.9±0.2 degrees 2-theta, and (b) a differential scanning calorimetry trace comprising a peak of from about 147° C. to about 151° C.

10. The method of claim 1, wherein the cancer in the individual is selected from the group consisting of metastatic brain cancer, breast cancer, and non-small cell lung cancer.

11. The method of claim 10, wherein the cancer in the individual is metastatic brain cancer.

12. The method of claim 10, wherein the cancer in the individual is non-small cell lung cancer.

13. The method of claim 1, wherein the cancer comprises one or more central nervous system (CNS) metastases.

14. The method of claim 1, wherein the cancer is non-small cell lung cancer and the individual is EGFR inhibitor naive prior to administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.

15. The method of claim 1, wherein the cancer is non-small cell lung cancer and the individual has received one or more prior EGFR inhibitor therapies for treatment of the cancer before administration to the individual of (R)—N-(2-(4-(4-cyclopropylpiperazin-1-yl)piperidin-1-yl)-5-((6-(3-(3,5-difluorophenyl)isoxazolidin-2-yl)pyrimidin-4-yl)amino)-4-methoxyphenyl)acrylamide, or a pharmaceutically acceptable salt thereof.
